(19) United States
(12) Patent Application Publication
(10) Pub. No.: US 2026/0078159 A1
(43) Pub. Date: Mar. 19, 2026

(54) NOVEL PD1-TARGETED IL-15 IMMUNOCYTOKINE and VITOKINE FUSIONS

(71) Applicant: Cugene Inc, Waltham, MA (US)

(72) Inventors: Yue-Sheng LI, Thousand Oaks, CA (US); Lingyun RUI, Weston, MA (US); Jing XU, Waltham, MA (US)

(21) Appl. No.: 19/110,034

(22) PCT Filed: Sep. 5, 2023
(86) PCT No.: PCT/US23/31967

§ 371 Date: Mar. 7, 2025

Related U.S. Application Data
(60) Provisional application No. 63/404,619, filed on Sep. 8, 2022.

(51) Int. Cl. C07K 14/54; A61K 38/00; A61K 39/00; A61P 35/04; C07K 14/715; C07K 16/28

(52) U.S. Cl. CPC C07K 14/5443; A61P 35/04; C07K 14/7155; C07K 16/2818; A61K 38/00; A61K 2039/505; C07K 2319/30

(57) ABSTRACT

The present disclosure provides novel PD1 Ab-IL-15 immunocytokine and VitoKine compositions that aim to target potency-attenuated or bio-activable IL-15 directly to tumor-infiltrating lymphocytes to reduce systemic mechanism-based toxicities and lead to broader therapeutic utility for IL-15 for the treatment of cancer. Potency attenuation of IL-15 in PD1 Ab-IL-15 immunocytokine improves target selectivity, facilitates the establishment of stoichiometric balance between the cytokine and antibody arms, and helps alleviate pathway over-activation and mitigate antigen sink and target-mediated deposition. IL-15 in PD1 Ab-IL-15 VitoKine will remain until activated locally by proteases that are upregulated in diseased tissues, this will prevent over-activation of the pathway and reduce undesirable “on-target” “off tissue” toxicities, and significantly decrease the potential antigen or target sink, and thus, prolong the in vivo half-life and result in improved biodistribution, bioavailability and therapeutic efficacy. In both PD1 Ab-IL-15 immunocytokine and VitoKine, PD1 antibodies capable of blocking PD1 and reversing T-cell anergy or exhaustion may further synergize with IL-15 anticancer immune response.

### RELATED APPLICATIONS

This application claims benefit of U.S. Provisional Application No. 63/404,619, filed on Sep. 8, 2022, incorporated in its entirety by reference herein.

### REFERENCE TO AN ELECTRONIC SEQUENCE LISTING

The contents of the electronic sequence listing (SeqListing-CUGENE PD1 AB-IL-15.xml; Size: 181 Kilobytes; Production Date: Sep. 5, 2023) is herein incorporated by reference in its entirety.

### TECHNICAL FIELD

While cancer has been traditionally treated by chemotherapy, radiation, targeted therapies and surgery, a fifth pillar of cancer treatment, immunotherapy, has emerged over the recent years and revolutionized the war on cancer. The benchmark for the immunotherapy drugs has been established by the development of T cell checkpoint (CTLA-4 and PD1/PD-L1) inhibitors. It has been demonstrated that these therapies effectively expand and reactivate the pool of tumor-specific T cells leading to objective response rates of up to 50% in patients with certain cancers.

Recently, interleukin-15 (IL-15), a member of the four α-helix bundle family of cytokines, has emerged as a candidate immunomodulator for the treatment of cancer. IL-15 binds to its specific receptor, IL-15Rα, and trans-activates a heterodimeric receptor complex composed of IL-15Rβ and the common cytokine receptor γ chain (γc) on the responding cells to initiate signaling. IL-15 exhibits broad activity and induces the differentiation and proliferation of T, B, and natural killer (NK) cells. It also enhances the cytolytic activity of CD8⁺ T cells and induces long-lasting antigen experienced CD8⁺CD44^{hi} memory T cells. IL-15 stimulates differentiation and immunoglobulin synthesis by B cells and induces maturation of dendritic cells. As such, it was hypothesized that boosting IL-15 activity could enhance innate and adaptive immunity and fight tumors, making it a promising agent for anticancer therapy (Steel et al., Trends Pharm Sci 33:35-41, 2012). Recombinant IL-15 and IL-15 in various fusion formats are tested in several on-going oncology clinical trials but have no approved uses to date.

Despite these new advancements using IL-15 as a cancer immunotherapeutic to augment immune responses, there remain limitations to the effective use of IL-15 as a therapeutic. For example, IL-15 has a short half-life (<40 minutes) resulting in 1) low bioavailability that impedes it's in vivo anti-tumor effects, and 2) the requirement for administration of a high dose to achieve therapeutic relevant exposure, which results in toxicity.

Several approaches have been taken to overcome the challenges inherent in IL-15 immunotherapy. One such approach to counter the systematic toxicity involves localizing cytokine activity to cancer cells and their surrounding tissues by tumor-targeted IL-15 immunocytokines, constructed by fusion of IL-15 to antibodies specific for tumor-associated antigens. However, this strategy lacks the ability to specifically target effector T cells within the tumor microenvironment (TME), which are pertinent to anticancer immunity. This gap in intratumoral T cell targeting may be filled by fusing IL-15 to an anti-programmed cell death protein 1 (PD1) antibody. PD1 (also known as CD279) is highly expressed on tumor-infiltrating lymphocytes (TILs), and PD1 antibody IL-15 immunocytokine enables IL-15 to be directly targeted to TILs. It displayed with elevated avidity toward intratumoral CD8+ T cells, rather than Treg cells or peripheral CD4+ and CD8+ T cells. This strategy thus further improves IL-15 anticancer immunity while reducing systemic toxicity.

In addition to targeting IL-15 directly to TILs to improve IL-15 anticancer immunity, PD1 antibodies capable of blocking PD1 and reversing T-cell anergy or exhaustion may synergize with IL-15, further boosting its anticancer immune response. Hence it is desirable to construct PD1 Ab-IL-15 immunocytokine with PD1 antibodies of superior target binding and PD1 blocking capabilities. Among the various globally marketed PD1 blocking antibodies, which have fundamentally transformed the field of cancer immunotherapy, pembrolizumab (Keytruda®; Merck Sharp & Dohme Corp.) has received remarkable attention due to its high effectiveness and approvals for treating a wide variety of cancer types. While pembrolizumab exhibits superior target binding and blocking capabilities, it has several sequence liabilities, including a relatively low degree of humanness that could raise immunogenicity concerns, and high hydrophobicity that tends to increase its aggregation propensity. It is thus preferable to optimize pembrolizumab to mitigate its sequence liabilities while fully maintaining its biological activity. The resulting optimized sequence is expected to improve the developability of PD1 Ab-IL-15 immunocytokines.

Importantly, fusion of a PD1 Ab with a fully active IL-15 moiety may override the intended antibody-mediated targeting, localizing the fusion protein to IL-15 receptor-expressing cells in the peripheral instead of TILs in tumors. As such, to improve target specificity and selectivity, one approach is to prepare a fusion using an IL-15 moiety with attenuated IL-15Rβγ activity to establish a stoichiometric balance between the cytokine and antibody components. Additionally, decreasing the cytokine potency can potentially alleviate pathway over-activation as well as mitigate antigen sink and target-mediated deposition.

Another related but more sophisticated strategy to improve target specificity and selectivity is the application of the VitoKine platform disclosed in WO2019246392 and WO2021119516 by the current inventors. In a VitoKine construct, the activity of the IL-15 moiety will remain inert or minimal until activated locally by proteases that are upregulated in or around tumors. By doing so, the binding of the IL-15 moiety to its receptors in the peripheral or on the cell-surface of non-diseased cells can be markedly limited. This can help prevent pathway over-activation and reduce undesirable “on-target” “off tissue” toxicity, and the improved safety profile of VitoKines may permit human dose levels within the effective range of a PD1 antibody. Additionally, the inertness of the IL-15 moiety prior to protease activation will significantly decrease the potential antigen or target sink, and thus, prolong the in vivo half-life and result in improved biodistribution and bioavailability at intended sites of therapy.

### DISCLOSURE OF THE INVENTION

In one aspect, the present invention provides a novel PD1-targeted bio-activable IL-15 immunocytokine (referred herein to as PD1 Ab-IL-15 VitoKine) that aims to target a bio-activable IL-15 directly to tumor-infiltrating lymphocytes. The activity of the IL-15 moiety will remain nearly inert or minimal until activated locally by proteases that are upregulated in tumors, which will limit binding of the IL-15 moiety to the receptors in the peripheral or on the cell-surface of non-diseased cells or normal tissues. This can help prevent pathway over-activation of the pathway and reduce undesirable “on-target” “off tissue” toxicity and minimize unwanted target sink.

In another aspect, the present invention provides a novel PD1 targeted-IL-15 immunocytokine that aims to target an activity-modulated IL-15 domain directly to tumor-infiltrating lymphocytes. The attenuated IL-15 activity is expected to facilitate establishing stoichiometric balance between the cytokine and antibody arms, help to alleviate pathway over-activation, and mitigate antigen sink and target-mediated deposition.

The strategy specifically targets effector T cells within the tumor microenvironment (TME) that are pertinent to anticancer immunity. By implementing this strategy, the ability of IL-15 to expand lymphocyte populations and augment their effector functions is synergized with the function of PD1 blocking antibody in reversing T-cell anergy or exhaustion. This approach, particularly when potency-attenuated or bio-activatable IL-15 is used, reduces systemic mechanism-based toxicities, leading to broader therapeutic utility of IL-15 for cancer treatment, and improve biodistribution and bioavailability at the intended sites of therapy.

In various embodiments, the PD1-targeted bio-activable IL-15 immunocytokine is referred to herein as PD1 Ab-IL-15 VitoKine. In various embodiments, the VitoKine platform disclosed in WO2019246392 and WO2021119516 by the current inventors is defined by the constructs as depicted in FIGS. 1A and 1B. In various embodiments, PD1 Ab-IL-15 VitoKine of the present invention is more specifically defined by the constructs illustrated in FIGS. 1C and 1D. Referring to FIG. 1C, the PD1 Ab-IL-15 VitoKine comprises a PD1 blocking antibody (targeting moiety domain; D1), a dimeric IL-15 domain (the active moiety domain; D2) with its N-terminus fused to the C-terminus of the homodimeric heavy chains of the PD1 antibody via the L1 linker and its C-terminus fused to the N-terminus of IL-15Rα sushi domains (the concealing moiety domain; D3) via the L2 linker. Referring to FIG. 1D, PD1 Ab-IL-15 VitoKine was constructed likewise except that the PD1 antibody contains a pair of knobs-into-holes heterodimeric heavy chains, and a monomeric IL-15 domain was fused to the knob heavy chain. In various embodiments, the proposed method of VitoKine activation is depicted in FIG. 2.

In various embodiments, the variable domains of the PD1 blocking antibodies of the present invention were optimized from the variable domains of pembrolizumab by introducing germline sequence substitutions to the CDR residues, introducing germline sequence substitutions to the framework somatic mutations, and/or adopting the most prevalent and better behaving VH3 human germline family sequence as the acceptor framework. In various embodiments, the PD1 blocking antibodies have a high affinity for the human PD1 protein as set forth in SEQ ID NO: 1, function to inhibit PD1 with equal or comparable potency as pembrolizumab, exhibit higher sequence similarity scores to its closest human germline sequence than pembrolizumab, thereby indicating an improved degree of humanness, and are predicted to have lower hydrophobicity than pembrolizumab, which in turn reduce their propensity to aggregate.

In various embodiments, the PD1 blocking antibody comprises a light chain variable region with the sequence set forth in SEQ ID NO: 3, and a heavy chain variable region with the sequence set forth in SEQ ID NO: 7. In various embodiments, the PD1 blocking antibody comprises a light chain variable region with the sequence set forth in SEQ ID NO: 3, and a heavy chain variable region with the sequence set forth in SEQ ID NO: 9. In various embodiments, the PD1 blocking antibody comprises a light chain variable region with the sequence set forth in SEQ ID NO: 3, and a heavy chain variable region with the sequence set forth in SEQ ID NO: 11. In various embodiments, the PD1 blocking antibody comprises a light chain variable region with the sequence set forth in SEQ ID NO: 3, and a heavy chain variable region with the sequence set forth in SEQ ID NO: 13. In various embodiments, the PD1 blocking antibody comprises a light chain variable region with the sequence set forth in SEQ ID NO: 3, and a heavy chain variable region with the sequence set forth in SEQ ID NO: 18.

In various embodiments, the PD1 targeted-IL-15 immunocytokine is defined by the constructs as depicted in FIG. 3. In various embodiments, the PD1 targeted-IL-15 immunocytokine comprise IL-15RαSushi+ domain with the sequence set forth in SEQ ID NO: 165 non-covalently complexed with IL-15. In various embodiments, the potency-modulated IL-15 of the PD1 targeted-IL-15 immunocytokine is an IL-15 variant (or mutant) comprising a sequence derived from the sequence of the mature human IL-15 polypeptide (also referred to herein as hulL-15 or IL-15 wild type (w/t) as set forth in SEQ ID NO: 116) comprising one or more amino acid substitution, deletion, or insertion. In various embodiments, the IL-15 variant demonstrates reduced signaling activity (EC₅₀ and/or Eₘₐₓ) compared to the native IL-15 polypeptide. The amino acid change can include one or more of an amino acid substitution, deletion, or insertion in the IL-15 polypeptide, such as in the domain of IL-15 that interacts with IL-15Rβ and/or the common cytokine receptor γ chain (γc). In various embodiments, the amino acid change is one or more amino acid substitutions at positions 30, 32, 63, 68, 108, 109 or 112 of SEQ ID NO: 116. In various embodiments, the amino acid change is the substitution of D to T at position 30, H to D or E or N or Q at position 32, V to F or A or K or R at position 63, I to F or H or D or K or Q or G at position 68, Q to A or D or E or F or H or K or L or M or N or S or T or Y at position 108, M to A or H or R at position 109, N to D or G or P or R at position 112 of the mature human IL-15 sequence, or any combination of these substitutions. In various embodiments, the amino acid change is 1, or 2, or 3, or 4 amino acid deletion at the N-terminus of SEQ ID NO: 116. In various embodiments, the amino acid change is 1, or 2, or 3, or 4, or 5, or 6, or 7, or 8, or 9, or 10 amino acid deletion at the C-terminus of SEQ ID NO: 116. In various embodiments, the IL-15 domain has any combinations of amino acid substitutions, deletions and insertions. In various embodiments, the potency-attenuated IL-15 moiety is selected from the group of sequences set forth in SEQ ID NOS: 117-163.

In various embodiments, the active moiety of the PD1 Ab-IL-15 VitoKine is an IL-15 domain comprising the sequence of the mature human IL-15 polypeptide as set forth in SEQ ID NO: 116. In various embodiments, the IL-15 domain is an IL-15 variant (or mutant) comprising a sequence derived from the sequence of the mature human IL-15 polypeptide as set forth in SEQ ID NO: 116 comprising one or more amino acid substitution, deletion, or insertion. In various embodiments, the IL-15 variant demonstrates increased signaling activity compared to the native IL-15 polypeptide. In various embodiments, the IL-15 variant demonstrates reduced signaling activity compared to the native IL-15 polypeptide. The amino acid change can include one or more of an amino acid substitution, deletion, or insertion in the IL-15 polypeptide, such as in the domain of IL-15 that interacts with IL-15Rβ and/or γc. In various embodiments, the amino acid change is one or more amino acid substitutions at positions 30, 32, 63, 68, 108, 109 or 112 of SEQ ID NO: 116. In various embodiments, the amino acid change is the substitution of D to T at position 30, H to D or E or N or Q at position 32, V to F or A or K or R, at position 63, I to F or H or D or K or Q or G at position 68, Q to A or D or E or F or H or Kor L or M or N or S or T or Y at position 108, M to A or H or R, at position 109, N to D or G or P or R, at position 112 of the mature human IL-15 sequence, or any combination of these substitutions. In various embodiments, the amino acid change is 1, or 2, or 3, or 4 amino acid deletion at the N-terminus of SEQ ID NO: 116. In various embodiments, the amino acid change is 1, or 2, or 3, or 4, or 5, or 6, or 7, or 8, or 9, or 10 amino acid deletion at the C-terminus of SEQ ID NO: 116. In various embodiments, the IL-15 domain has any combinations of amino acid substitutions, deletions and insertions. In various embodiments, VitoKine construct utilizes an IL-15 variant having optimally attenuated potency thus leading to diminished intrinsic basal activity of the corresponding VitoKine construct. In various embodiments, the IL-15 variant in the VitoKine construct can tune the IL-15 VitoKine intrinsic basal activity to achieve an optimal balance between desired anti-tumor efficacy and unwanted systematic toxicity. In various embodiments, the IL-15 domain of PD1 Ab-IL-15 VitoKine is selected from the group of sequences set forth in SEQ ID NOS: 116-163.

In various embodiments, the concealing moiety domain of PD1 Ab-IL-15 VitoKine is a cognate receptor/binding partner, or any binding partner identified for the IL-15. In various embodiments, the concealing moiety domain is an IL-15Rα extracellular domain or a functional fragment thereof. In various embodiments, the IL-15Rα extracellular domain or a functional fragment thereof is an IL-15RαSushi+ domain with the sequence set forth in SEQ ID NO: 165.

In various embodiments, L1 linker and L2 linker of PD IL-15 VitoKine are both a protease cleavable peptide linker. In various embodiments, L1 linker is a protease cleavable peptide linker and L2 is a non-cleavable peptide linker. In various embodiments, L1 linker is a non-cleavable peptide linker and L2 is a protease cleavable peptide linker. In various embodiments, L1 linker and L2 linker of the PD1 Ab-IL-15 VitoKine constructs are both a protease non-cleavable peptide linker. In various embodiments, the non-cleavable linker is rich in G/S content (e.g., at least about 60%, 70%, 80%, 90%, or more of the amino acids in the linker are G or S. Each peptide linker sequence can be selected independently. In various embodiments, the protease cleavable linker is selected from the group of sequences set forth in SEQ ID NOS: 54-77. In various embodiments, the protease cleavable linker can have additional peptide spacer of variable length on the N-terminus of the cleavable linker or on the C-terminus of the cleavable linker or on both termini of the cleavable linker. In various embodiments, L1 linker and L2 linker of the PD1 Ab-IL-15 VitoKine constructs are both a protease non-cleavable peptide linker. In various embodiments, the protease cleavable linker with peptide spacer of variable length on either the N-terminus or on the C-terminus or on both termini of the cleavable linker is selected from the group of sequences set forth in SEQ ID NOS: 78-94. In various embodiments, the non-cleavable linker is selected from the group of sequences set forth in SEQ ID NOS: 95-115. In various embodiments, the linker is either flexible or rigid and of a variety of lengths.

In various embodiments, the IL-15 domain (D2) and IL-15Rα domain (D3) of the VitoKine construct are placed at the C-terminus of the PD1 Ab domain (D1) as depicted in FIG. 1A. In various embodiments, the D2 and D3 domains of the VitoKine construct are placed at the N-terminus of the PD1 Ab domain (D1) domain as depicted in FIG. 1B.

In various embodiments, the PD1 blocking Ab, IL-15 domain and IL-15Rα domain of PD1 Ab-IL-15 VitoKine construct can be monomer, or dimer (illustrated in FIG. 1C), or a combination of dimer and monomer, such as PD1 blocking Ab is dimer and IL-15 domain and IL-15Rα domains are monomer (illustrated in FIG. 1D).

In another aspect, the present disclosure provides a method for treating cancer or cancer metastasis in a subject, comprising administering a therapeutically effective amount of the pharmaceutical compositions of the invention to a subject in need thereof. In one embodiment, the subject is a human subject. In various embodiments, the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.

In another aspect, the present disclosure provides a method for treating cancer or cancer metastasis in a subject, comprising administering a therapeutically effective amount of the pharmaceutical compositions of the invention in combination with a second therapy selected from the group consisting of: cytotoxic chemotherapy, immunotherapy, small molecule kinase inhibitor targeted therapy, surgery, radiation therapy, stem cell transplantation, cell therapies including chimeric antigen receptor (CAR)-T, CAR-NK, induced pluripotent stem cells (IPS) induced CAR-T or iPS induced CAR-NK and vaccine such as Bacille Calmette-Guerine (BCG). In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-L1, CD40, OX-40, CD137, GITR, LAG3, TIM-3, Siglec-7, Siglec-8, Siglec-9, Siglec-15 and VISTA; treatment using bispecific T cell engaging antibodies (BiTE®) such as blinatumomab: treatment involving administration of biological response modifiers such as IL-12, IL-151, GM-CSF, IFN-□□□IFN-β and IFN-γ; treatment using therapeutic vaccines such as sipuleucel-T; treatment using dendritic cell vaccines, or tumor antigen peptide vaccines; treatment using CAR-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; and treatment using vaccine such as BCG; wherein the combination therapy provides increased effector cell killing of tumor cells, i.e., a synergy exists between the pharmaceutical compositions of the invention and the immunotherapy when co-administered.

In another aspect, the disclosure provides uses of the pharmaceutical compositions of the invention for the preparation of a medicament for the treatment of cancer.

In another aspect, the present disclosure provides isolated nucleic acid molecules comprising a polynucleotide encoding a pharmaceutical composition of the invention of the present disclosure. In another aspect, the present disclosure provides vectors comprising the nucleic acids described herein. In various embodiments, the vector is an expression vector. In another aspect, the present disclosure provides isolated cells comprising the nucleic acids of the disclosure. In various embodiments, the cell is a host cell comprising the expression vector of the disclosure. In another aspect, methods of making the pharmaceutical compositions of the invention are provided by culturing the host cells under conditions promoting expression of the proteins or polypeptides.

In another aspect, the present disclosure provides a pharmaceutical composition comprising the isolated pharmaceutical compositions of the invention in admixture with a pharmaceutically acceptable carrier.

### BRIEF DESCRIPTION OF THE FIGURES

FIG. 1 depicts representative VitoKine construct formats. (A) VitoKine construct with the active moiety domain (D2) and concealing moiety domain (D3) being placed at the C-terminus of the targeting domain (D1). (B) VitoKine construct with the D2 and D3 domains being placed at the N-terminus of the D1 domain. (C) Representative PD1 Ab dimeric IL-15 VitoKine format. (D) Representative PD1 Ab monomeric IL-15 VitoKine format.

FIG. 2 depicts the proposed activation mechanism for the PD1 Ab-IL-15 VitoKine constructs. The exemplary VitoKine construct comprises two protease cleavable linkers; protease 1 activation resulted from cleavage of L1 linker yields Active Form 1; protease 2 activation resulted from cleavage of L2 linker yields Active Form 2; activation by both proteases resulted from cleavage of L1 and L2 linkers yields Active Form 3. Following protease cleavage, the IL-15RαSushi domain (D3) remains non-covalently complexed with IL-15 domain (D2). If L1 linker is the only protease cleavable linker, then Active Form 1 will be the sole activated format. Similarly, if L2 linker is the only protease-cleavable linker, then Active Form 2 will be the singular activated format.

FIG. 3 depicts the structures of PD1-targeted IL-15 immunocytokines with the IL-15 domain as either monomeric (A) or dimeric (B), and the structures of IL-15 Fc fusion proteins with the IL-15 domain as either monomeric (C) or dimeric (D). All configurations comprise IL-15Rα non-covalently complexed with IL-15.

FIG. 4 depicts a comparison of the PD1 blocking activity between the Reference Antibody (P-0734) and pembrolizumab (PBL) biosimilar in a luciferase reporter assay. FIG. 4A and FIG. 4B depict dose-dependent increases in luminescence signal and fold induction, respectively. P-0734 and PBL biosimilar share the identical variable domains and have IgG1 and IgG4 isotypes, respectively.

FIG. 5 depicts (A) ELISA binding and (B-C) PD1 blockade activity of PD1 blocking antibodies, P-1148, P-1150, P-1151, and P-1153, compared to the Reference Antibody (P-0734), as tested in a luciferase reporter assay. FIG. 5B and FIG. 5C depict dose-dependent increases in luminescence signal and fold induction, respectively.

FIG. 6 depicts PD1 blockade activity of PD1 blocking antibodies, P-1127, P-1129, and P-1174, compared to the Reference Antibody (P-0734). They were tested in a luciferase reporter assay and the dose-dependent increases in luminescence signal are illustrated.

FIG. 7 depicts PD1 blockade activity of PD1 blocking antibodies, P-1175 and P-1181, compared to the Reference Antibody (P-0734. FIG), as tested in a luciferase reporter assay. 7A and FIG. 7B depict dose-dependent increases in luminescence signal and fold induction, respectively.

FIG. 8 depicts PD1 blockade activity of PD1 blocking antibodies, P-1175, P-1176, P-1177, and P-1178, compared to the Reference Antibody (P-0734), as tested in a luciferase reporter assay. FIG. 8A and FIG. 8B depict dose-dependent increases in luminescence signal and fold induction, respectively.

FIG. 9 depicts PD1 blockade activity of PD1 blocking antibodies, P-1198, P-1199, and P-1201, compared to the Reference Antibody (P-0734), as tested in a luciferase reporter assay. FIG. 9A and FIG. 9B depict dose-dependent increases in luminescence signal and fold induction, respectively. A non-targeting germline antibody was included as the negative control.

FIG. 10 depicts PD1 blockade activity of PD1 blocking antibodies, P-1194, P-1201, and P-1238, compared to the Reference Antibody (P-0734), as tested in a luciferase reporter assay. FIG. 10A and FIG. 10B depict dose-dependent increases in luminescence signal and fold induction, respectively.

FIG. 11 depicts the binding of PD1 blocking antibodies, P-1174, P-1193, P-1198, P-1199, and P-1201, to PD1+ HEK293 cells, compared to the Reference Antibody (P-0734).

FIGS. 11A and 11C depict dose-dependent increases in percentage of positive cells, and FIGS. 11B and 11D depict dose-dependent increases in mean fluorescence intensity (MFI).

FIG. 12 depicts the activity assessment of P-0234 and P-0313 by analyzing their effects on the induction of Ki67 expression in CD8+ T cells of fresh human PBMCs using flow cytometry. P-0234 and P-0313 are dimeric IL-15 Fc fusion proteins comprising wildtype IL-15 and S58D variant, respectively. A recombinant Fc protein serves as the negative control.

FIG. 13 depicts the activity assessment of various IL-15 variants by analyzing their effect on the induction of Ki67 expression in CD8+ T cells of fresh human PBMCs. These IL-15 variants comprise amino acid substitutions targeting its interaction with IL-15Rβ, including A) single amino acid substitutions at position 168, B) single amino acid substitutions at position V63, and C) combinational mutations at positions V63 and 168 along with their counterparts with individual amino acid alterations. P-0313, a well-characterized dimeric IL-15 S58D Fc fusion protein, acts as the dimeric wildtype IL-15 control.

FIG. 14 depicts the activity assessment of various IL-15 deletion mutants by analyzing their effect on the induction of Ki67 expression in CD8+ T cells of fresh human PBMCs. P-0866, P-0867, and P-0868 comprise 1, 2, and 3 amino acid deletion at the N-terminus of IL-15, respectively. P-0234 is the dimeric wildtype IL-15 control.

FIG. 15 depicts the comparison of the effects of IL-15 variants on (A) induction of Ki67 expressions in CD8+ T cells of fresh human PBMCs, and (B) sustaining the proliferation of mouse-derived CTLL-2 cells. These IL-15 variants comprise either single amino acid substitution at position V63 (P-0771) or 168 (P-0737) or combinational mutations at positions V63 and 168 (P-0768, P-0772 and P-0773).

FIG. 16 depicts the comparison of the effects of IL-15 variants on (A) induction of Ki67 expressions in CD8+ T cells of fresh human PBMCs, and (B) sustaining the proliferation of CTLL-2 cells. P-0867 and P-0868 comprise 2 and 3 amino acid deletion at the N-terminus of IL-15, respectively. P-0234 is the dimeric wildtype IL-15 control.

FIG. 17 depicts the activity assessment of various IL-15 variants by analyzing their effect on the induction of Ki67 expression in CD8+ T cells of fresh human PBMCs. These variants comprise acid substitutions at the Q108 position targeting its interaction with the common γ receptor (γc), including A-C) single amino acid substitutions at Q108, and D) Q108N mutation combined with another amino acid change at V63 or 168 that interferes with the IL-15Rβ interface. P-0217 is the monomeric wildtype 15 control.

FIG. 18 depicts the comparison of the effects of IL-15 variants on (A) induction of Ki67 expressions in CD8+ T cells of fresh human PBMCs, and (B) sustaining the proliferation of CTLL-2 cells. These IL-15 variants contain the Q108M mutation to interrupt interaction with γc, along with another amino acid change at V63 or 168 that interferes with the IL-15Rβ interface. P-0217 is the monomeric wildtype 15 control.

FIG. 19 depicts the activity assessment of IL-15 variants by analyzing their effect on the induction of Ki67 expression in CD8+ T cells of fresh human PBMCs. These variants comprise acid substitutions at the N112 position targeting its interaction with γc. P-0217 is the monomeric wildtype 15 control.

FIG. 20 depicts the comparison of the activity of IL-15 variants in distinct fusion formats, specifically Fc fusion and PD-1 Ab fusion, based on their effect on the induction of Ki67 expression on CD8+ T cells of fresh human PBMCs. (A) Both P-0773 and P-0870 are dimeric IL-15 V63A/168H variant fusion proteins; P-0773 is an Fc fusion while P-0870 is a PD1Ab fusion. (B) P-0867 and P-0888 are a similar pair of Fc and antibody fusions, each comprising 2-amino acid deletion at the N-terminus of IL-15. P-0234 and P-0313 interchangeably serve as the dimeric wildtype IL-15 control.

FIG. 21 depicts a comparison between P-1352, a PD1 Ab-IL15 immunocytokine, and P-1271, the PD1 blocking component of P-1352, in terms of A) PD-1 binding and B) PD-L1 binding inhibition using two ELISA assays. P-1260, a non-targeting germline antibody, was used as a negative control.

FIG. 22 depicts the impact of IL-15 valency in the activity of PD1 Ab-IL-15 immunocytokines by analyzing their effect on inducing Ki67 expression in CD8+ T cells of fresh human PBMCs. P-0869 is a PD1 Ab-IL15 immunocytokine containing a dimeric IL-15 V63AA/168H variant, while P-1266 is its monomeric IL-15 equivalent.

FIG. 23 depicts the ex vivo activity comparison among the three mouse PD1 Ab-IL15 immunocytokines, P-1266, P-1295, and P-1296, by analyzing Ki67 expression in CD8+ T cells of fresh human PBMCs. They contain monomeric IL-15 variants with V63A/168H, Q108N, and 168H/Q108N mutations, respectively and display varying degrees of reduced activity. P-1284 serves as the format-matched control featuring monomeric wildtype IL-15.

FIG. 24 depicts the comparison of two mouse PD1 Ab-IL15 immunocytokines, P-1266 and P-1295, based on their effects on peripheral lymphocytes in increasing A) Ki67 expression in CD8 T cells, B) CD8 T cell numbers, C) granzyme B expression in CD8 T cells, D) Ki67 expression in NK cells, and E) NK cell numbers in C57B/L6 mice following a single intraperitoneal injection. Blood samples were collected on Days 0, 5, 7, 10, and 10 for lymphocyte phenotyping using FACS analysis. The comparative effects of these two immunocytokines on the body weight of mice are illustrated in FIG. 24F. Data are presented as the mean±the standard error of the mean (SEM).

FIG. 25 depicts a comparative analysis of the serum concentrations of two mouse PD1 Ab-IL15 immunocytokines, P-1266 and P-1295, following a single intraperitoneal injection in C57B/L6 mice. P-1266 and P-1296 contain monomeric IL-15 variants with V63A/168H and 168H/Q108N mutations, respectively. Blood samples were taken at multiple time points post-dosing and the serum concentrations of the compounds were determined using an ELISA assay.

FIG. 26 depicts the comparison of the pharmacodynamic effects of P-1266 at a 1.5 mg/kg (mpk) dose and P-1296 at 1.5 and 3.0 mpk doses on peripheral lymphocytes in MC38 tumor-bearing mice. P-1266 and P-1296 are mouse PD1 Ab-IL-15 immunocytokine containing monomeric IL-15 variants with V63A/168H and 168H/Q108N mutations, respectively. Blood samples for analysis were taken 5 days after a single intraperitoneal injection to assess A) the increase in Ki67 expression in CD8 T cells, B) the increase in Ki67 expression in NK cells, C) the expansion of CD8 T cells, and D) the expansion of NK cells. Each group consisted of 4 mice.

FIG. 27 depicts the comparison of the pharmacodynamic effects of P-1266 and P-0869 at a 1.5 mpk dose on peripheral lymphocytes in MC38 tumor-bearing mice. P-0869 is a mouse PD1 Ab-IL15 immunocytokine containing a dimeric IL-15 V63AA/168H variant, while P-1266 is its monomeric IL-15 equivalent. Blood samples for analysis were taken 5 days after a single intraperitoneal injection to assess A) the increase in Ki67 expression in CD8 T cells, B) the increase in Ki67 expression in NK cells, C) the expansion of CD8 T cells, and D) the expansion of NK cells. Each group consisted of 4 mice.

FIG. 28 depicts the dose-dependent anti-tumor efficacy of P-0869, a mouse PD1 Ab-IL-15 immunocytokine comprising dimeric IL-15 V63A/168H variant, in both CT26 and MC38 murine tumor models. FIG. 28A displays the mean CT26 tumor volume±SEM over time for various treatment groups, following two Q10D doses at varying dosing levels (0.3, 1.0, and 2.0 mg/kg). Additionally, FIG. 28A also showcases the absence of tumor recurrence after rechallenge implantation of CT26 cells in previously tumor-free mice. This was contrasted with the successful regrowth of the same type of tumor in age-matched naïve mice as a control. FIG. 28B illustrates the mean MC38 tumor volume±SEM over time for each treatment group following two Q12D doses at 0.3 and 1.0 mg/kg. In both tumor models, a Vehicle group was included for reference.

FIG. 29 depicts the comparison of the anti-tumor efficacy of P-0869 and P-1266, which are the dimeric and monomeric pair of the mouse PD1 Ab-IL 15 V63A/168H immunocytokines, in both CT26 and MC38 murine tumor models. The mean tumor volume±SEM over time for each group following two Q12D doses at 1.5 mpk is illustrated for A) the CT26 model and B) the MC38 model. In both tumor models, a Vehicle group was included for reference.

FIG. 30 depicts the comparison of the anti-tumor efficacy of mouse PD1 Ab-IL-15 immunocytokines, P-1266 and P-1295, which contain monomeric IL-15 variants with V63A/168H and Q108N mutations, respectively. The mean tumor volume±SEM over time for each group following two Q12D doses at 1.5 mpk is illustrated for A) the CT26 model and B) the MC38 model. In both tumor models, a Vehicle group was included for reference.

FIG. 31 depicts the anti-tumor efficacy of P-1296, a mouse PD1 Ab-IL-15 immunocytokine containing a monomeric IL-15 168H/Q108N variant, following two Q12D doses at different dosing levels. The mean tumor volume±SEM over time for each group is illustrated for A) the CT26 model with dosages of 1.5 and 3.0 mpk and B) the MC38 model with dosages of 1.0 and 3.0 mpk. Both tumor studies include a Vehicle group for comparative purposes.

FIG. 32 depicts the assessment of IL-15 VitoKine platform's ability to conceal its functionality by comparing the activity of VitoKines to their counterpart non-VitoKine fusion proteins in a human PBMC assay using flow cytometry. Exemplary Fc VitoKine (P-0315) and PD1 Ab VitoKine (P-875) and their respective non-VitoKine counterparts, P-0313 and P-0870, were tested for stimulating Ki67 expression on CD8+ T cells (A & C) and CD56+NK cells (B &D).

FIG. 33 depicts the size exclusion chromatograms of five PD1 Ab-IL-15 VitoKines (P-0874, P-1077, P-1083, P-1084, and P-1085) in comparison to that of their non-VitoKine counterpart, P-0869. The five VitoKines differ only in the lengths and compositions of the L2 linker connecting the IL-15 and IL-15RαSushi+ domains.

FIG. 34 depicts the dose-dependent induction of Ki67 expression on A) CD8+ T cells and B) NK cells following treatment with PD1 Ab-IL-15 VitoKines in fresh human PBMCs. The five PD1 Ab-IL-15 VitoKines (P-0874, P-1077, P-1083, P-1084, and P-1085) differ only in the lengths and compositions of the L2 linker. P-0869 is their non-VitoKine counterpart.

FIG. 35 depicts a side-by-side evaluation of the activity of PD1 Ab-IL-15 VitoKines, P-1265 and P-1263, in comparison to their corresponding non-VitoKine counterparts, P-1266 and P-1295. This is based on their effect on the induction of Ki67 expression on A) CD8+ T cells and B) NK cells of fresh human PBMCs. P-1284 serves as the format-matched control featuring monomeric wildtype IL-15.

FIG. 36 depicts a comparison between P-1340, a PD1 Ab-IL15 VitoKine, and P-1271, the PD1 blocking component of P-1340, in terms of A) PD-1 binding and B) PD-L1 binding inhibition using two ELISA assays. P-1260, a non-targeting germline antibody, was used as a negative control.

FIG. 37 depicts the protease cleavage and activation of PD1 Ab-IL-15 VitoKine P-0875 with flow cytometry analysis of dose-dependent induction of Ki67 expression on A) CD8+ T cells and B) NK cells in human PBMCs, as well as C) reduced SDS-PAGE gel analysis. P-0875 and P-0870 are PD1 Ab VitoKine and non-VitoKine counterpart pairs containing dimeric IL-15 V63A/168H variant.

FIG. 38 depicts a comparative analysis of the activity of a mouse PD1 Ab-IL-15 VitoKine, P-1265, at vary dosing levels (3, 6, and 12 mpk), relative to its non-VitoKine counterpart, P-1266, dosed at 1.5 mpk, in C57B/L6 mice. The comparison is based on their effects on peripheral lymphocytes in increasing A) Ki67 expression in CD8 T cells, B) CD8 T cell numbers, C) Ki67 expression in NK cells, and D) NK cell numbers following a single intraperitoneal injection. Blood samples were collected on Days 0, 3, 5, 7, 10, and 10 for lymphocyte phenotyping using FACS analysis. Data are presented as the mean±the standard error of the mean (SEM).

FIG. 39 depicts a comparative analysis of the activity of the mouse PD1 Ab-IL-15 VitoKine, P-1265, relative to its dimeric VitoKine equivalent, P-1085, and its non-VitoKine counterpart, P-1266, in C57B/L6 mice. The comparison is based on their effects on peripheral lymphocytes in increasing A) Ki67 expression in CD8 T cells, B) CD8 T cell numbers, C) Ki67 expression in NK cells, and D) NK cell numbers following a single intraperitoneal injection. The dosages for the two VitoKines were 12 mpk and the dosage for P-1266 was 1.5 mpk. Blood samples were collected on Days 0, 3, 5, 7, 10, and 10 for lymphocyte phenotyping using FACS analysis. Data are presented as the mean±the standard error of the mean (SEM).

FIG. 40 depicts a comparative analysis of the activity of the mouse PD1 Ab-IL-15 VitoKine, P-1263, at vary dosing levels (6, 12, and 24 mpk), relative to its non-VitoKine counterpart, P-1295, dosed at 1.5 mpk, in C57B/L6 mice. The comparison is based on their effects on peripheral lymphocytes in increasing A) Ki67 expression in CD8 T cells, B) CD8 T cell numbers, C) Ki67 expression in NK cells, and D) NK cell numbers following a single intraperitoneal injection. Blood samples were collected on Days 0, 3, 5, 7, 10, and 10 for lymphocyte phenotyping using FACS analysis. Data are presented as the mean±the standard error of the mean (SEM).

FIG. 41 depicts a comparative analysis of the activity of the mouse PD1 Ab-IL-15 VitoKine, P-1263, relative to its non-cleavable VitoKine equivalent, P-1264, and its non-VitoKine counterpart, P-1295, in C57B/L6 mice. The comparison is based on their effects on peripheral lymphocytes in increasing A) Ki67 expression in CD8 T cells, B) CD8 T cell numbers, C) Ki67 expression in NK cells, and D) NK cell numbers following a single intraperitoneal injection. The dosages for the two VitoKines were 12 mpk and the dosage for P-1295 was 1.5 mpk. Blood samples were collected on Days 0, 3, 5, 7, 10, and 10 for lymphocyte phenotyping using FACS analysis. Data are presented as the mean±the standard error of the mean (SEM).

FIG. 42 depicts P-0874 (a mouse PD1 Ab-IL-15 VitoKine)'s in vivo anti-tumor efficacy and pharmacodynamic effects in mice with established CT26 murine tumors, compared to its non-cleavable VitoKine counterpart, P-0878, following two Q12D doses of 10 mg/kg. The analysis includes A) mean tumor volume±SEM over time for each treatment group, B) expansion of CD8 T cells 5 days post-dosing, and C) expansion of NK cells 5 days post-dosing.

FIG. 43 depicts a comparative analysis of the anti-tumor efficacy of the mouse PD1 Ab-IL-15 VitoKine, P-1265, relative to its dimeric VitoKine equivalent, P-1085, and their respective non-VitoKine counterparts, P-1266 and P-0869, in an established MC38 tumor model. The analysis includes mean tumor volume±SEM over time for A) P-1085 dosed at 3 and 6 mpk and P-0869 at 1.5 mpk, and B) P-1265 dosed at 3, 6, and 12 mpk and P-1266 at 1.5 mpk. Along with a Vehicle group, the component PD1 antibody, P-0722, dosed at 12 mpk was included for comparison.

FIG. 44 depicts the anti-tumor effects of P-1263, a mouse PD1 Ab-IL-15 VitoKine, in an established MC38 murine colon carcinoma model after two Q12D doses. The component mouse PD1 antibody, P-0722, administered at dosages of 6 and 18 mpk, was included for comparative analysis. The mean MC38 tumor volume±SEM over time for each treatment group is illustrated in FIG. 44A. The growth curve of MC38 tumors in individual mice is presented for B) P-0722 at 6 mg/kg, C) P-722 at 18 mg/kg, D) P-1263 at 6 mg/kg, E) P-1263 at 9 mg/kg, and F) P-1263 at 18 mg/kg. For comparison, the mean tumor volume±SEM over time for the Vehicle group is plotted with a dotted line. The change in body weight over time for each treatment group is shown in FIG. 44G.

### MODE(S) FOR CARRYING OUT THE DISCLOSURE

In one aspect, the present invention provides PD1 Ab-IL-15 VitoKine constructs comprising an optimized PD1 blocking antibody as the TIL-targeting moiety, an IL-15 or IL-15 variant as the active moiety domain, and an IL-15 RαSushi domain as the concealing moiety domain. Importantly, the IL-15 RαSushi domain is capable of concealing or attenuating the functional activity of IL-15 domain until activated at the intended site of therapy.

The PD1 blocking antibody guides the VitoKine to the TILs in the tumor microenvironment and restrict the activation of the VitoKine locally to improve the therapeutic index. The PD1 antibody were optimized from the variable domains of pembrolizumab by germline sequence substitutions of the CDR residues, germline sequence substitutions of the framework somatic mutations, adoption of the most prevalent and better behaving VH3 human germline family sequence as the acceptor framework. have a high affinity for PD1, function to inhibit PD1 with equal or comparable potency as pembrolizumab, have higher sequence similarity score to its closest human germline sequence consequently improved degree of humanness than pembrolizumab, are predicted to have lower hydrophobicity than pembrolizumab, and consequently lowered aggregation propensity. The PD1 blocking antibody with the optimized sequences are expected to improve the developability properties of the PD1 IL-15 immunocytokines.

In another aspect, the IL-15 domain in PD1 Ab-IL-15 VitoKine constructs is an active moiety but remains inert until activated locally by proteases that are upregulated in diseased tissues; this will limit binding of the active moiety to the receptors in the peripheral or on the cell-surface of non-diseased cells or tissue to prevent over-activation of the pathway and reduce undesirable “on-target” “off tissue” toxicity. Additionally, the inertness of the VitoKine active moiety prior to protease activation will significantly decrease the potential antigen sink, and thus, prolong the in vivo half-life and result in improved biodistribution, bioavailability and efficacy at intended sites of therapy.

In various embodiments, the integration of a potency-attenuated IL-15 variant as the active moiety domain (such IL-15 variant achieved by disrupting IL-15Rβγ interaction) can further fine-tune the intrinsic basal activity and post-activation activity of the VitoKine. In various embodiments, such VitoKine with potency attenuated IL-15 variant as the active moiety domain may additionally expand the therapeutic index.

The unique and non-signaling α-subunit of receptors of IL-15 is used as the concealing moiety domain via a protease-cleavable linker to reversibly conceal the cytokine activity in PD1 Ab-IL-15 VitoKine. The concealing α-subunit may preferably be complexed with the activated cytokine through non-covalent association after protease cleavage of the linker.

The three domains in PD1 Ab-IL-15 VitoKine constructs are linked using linkers having variable length and rigidity coupled with protease cleavable sequences, which are peptide substrates of specific protease subtypes with elevated or dysregulated expression in the disease sites, thus allowing for a functional IL-15 domain to be revealed or released at the site of disease. The linker length and composition were optimized to drive the best concealing of the accessibility of IL-15 domain to its receptors to reduce its systemic engagement, while maintaining the stability of the VitoKines in the blood circulation and allowing efficient cleavage after encountering specific proteases at intended site of disease.

In another aspect, the present disclosure provides novel PD1 targeted IL-15 immunocytokines that aim to target an activity-modulated IL-15 domain directly to tumor-infiltrating lymphocytes. In various embodiments, the activity-modulated IL-15 domain (dimeric) is fused to the C-terminus of the PD1 antibody heavy chain. In various embodiments, the activity-modulated IL-15 domain (monomeric) is fused to the C-terminus of the heterodimeric PD1 antibody heavy chain. In various embodiments, the PD1 targeted-IL-15 immunocytokine comprise IL-15RαSushi+ domain with the sequence set forth in SEQ ID NO: 165 non-covalently complexed with IL-15.

In one aspect, the IL-15 domain in PD1 targeted IL-15 immunocytokine has attenuated IL-15Rβγ activity and is expected to facilitate establishing stoichiometric balance between the cytokine and antibody arms, help to alleviate pathway over-activation, and mitigate antigen sink and target-mediated deposition. In various embodiments, use of a potency-attenuated IL-15 variant (such variant having impaired interaction with γc) in the PD1 targeted IL-15 immunocytokine could offer additional benefits in mitigating antigen sink and in turn result in an extend in vivo half-life likely because of the impact of γc receptor in the signaling cascade leading to cell expansion.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (amino group when participating in a peptide bond) of an amino acid at any other location within the peptide. Similarly, the term “carboxy terminus” (abbreviated C-terminus) refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties.

An amino acid “substitution” as used herein refers to the replacement in a polypeptide of one amino acid at a particular position in a parent polypeptide sequence with a different amino acid. Amino acid substitutions can be generated using genetic or chemical methods well known in the art. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine(S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.±0.1); glutamate (+3.0.±0.1); serine (+0.3); asparagine (+0.2); glutamine (+0.2); glycine (0); threonine (−0.4); proline (−0.5.±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within ±2 is included, in various embodiments, those that are within ±1 are included, and in various embodiments, those within ±0.5 are included.

Exemplary amino acid substitutions are set forth in Table 1.

TABLE 1

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant”, “hybrid polypeptide” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Natl. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “modification” as used herein refers to any manipulation of the peptide backbone (e.g., amino acid sequence) or the post-translational modifications (e.g., glycosylation) of a polypeptide.

The term “knob-into-hole modification” as used herein refers to a modification within the interface between two immunoglobulin heavy chains in the CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001).

The term “bioactivatable drug” or “VitoKine” as used herein means a compound that is a drug precursor which, following administration to a subject, releases the drug in vivo via some chemical or physiological process such that the bioactivatable drug is converted into a product that is active to the target tissues. A bioactivatable drug is any compound that undergoes bioactivation before exhibiting its pharmacological effects. Bioactivatable drugs can thus be viewed as drugs containing specialized non-toxic protective groups used in a transient manner to alter or to eliminate undesirable properties in the parent molecule.

The term “immunoconjugate” or “fusion protein” as used herein refers to a molecule comprising an antibody or antigen-binding fragment thereof conjugated (or linked) directly or indirectly to an effector molecule. The effector molecule can be a detectable label, an immunotoxin, cytokine, chemokine, therapeutic agent, or chemotherapeutic agent. The antibody or antigen-binding fragment thereof may be conjugated to an effector molecule via a peptide linker. An immunoconjugate and/or fusion protein retains the immunoreactivity of the antibody or antigen-binding fragment, e.g., the antibody or antigen-binding fragment has approximately the same, or only slightly reduced, ability to bind the antigen after conjugation as before conjugation. As used herein, an immunoconjugate may also be referred to as an antibody drug conjugate (ADC). Because immunoconjugates and/or fusion proteins are originally prepared from two molecules with separate functionalities, such as an antibody and an effector molecule, they are also sometimes referred to as “chimeric molecules.”

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded, digested, or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The term “peptide linker” as used herein refers to a peptide comprising one or more amino acids, typically about 1-30 amino acids. Peptide linkers are known in the art or are described herein. Suitable, non-immunogenic linker peptides include, for example, (G₄S)ₙ, (SG₄)ₙ or G₄(SG₄) n peptide linkers. “n” is generally a number between 1 and 10, typically between 2 and 4.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

The term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD1, PDL-1, CD40, OX-40, CD137, GITR, LAG3, TIM-3, SIRPα, CD47, GITR, ICOS, CD27, Siglec 7, Siglec 8, Siglec 9, Siglec 15, VISTA, CD276, CD272, TIM-3, and B7-H4; treatment using bispecific T cell engaging antibodies (BiTE®) such as blinatumomab: treatment involving administration of biological response modifiers such as IL-15, IL-4, IL-7, IL-10, IL-12, IL-15, IL-151, IL-152, GM-CSF, IFN-□□□IFN-β and IFN-γ, TGF-β antagonist or TGF-β trap; treatment using therapeutic vaccines such as sipuleucel-T; treatment using therapeutic virus, including, but not limited to oncolytic virus such as T-vec; treatment using dendritic cell vaccines, or tumor antigen peptide or neoantigen vaccines; treatment using NK cells; treatment using chimeric antigen receptor (CAR)-T cells; treatment using CAR-NK cells; treatment using DC or T cells; treatment using treatment using iPS induced-NK cells; treatment using iPS induced-T cells, and treatment using vaccine such as Bacille Calmette-Guerine (BCG); treatment using tumor infiltrating lymphocytes (TILs); treatment using adoptively transferred anti-tumor T cells (ex vivo expanded and/or TCR-T cells); treatment using TALL-104 cells; and treatment using immunostimulatory agents such as Toll-like receptor (TLR) agonists CpG, TLR7, TLR8, TLR9, and imiquimod.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor.

The term “neoantigen” refers to, e.g., cell surface antigens to which the immune system has not previously been exposed, especially one that arises by alteration of host antigens by radiation, chemotherapy, viral infection, neoplastic transformation/mutation, drug metabolism, etc., selectively expressed by cancer cells or over-expressed in cancer cells relative to most normal cells.

The term “antibody” as used herein is used in the broadest sense and encompasses various antibody structures (IgG1, 2, 3, or 4, IgM, IgA, IgE) including but not limited to monoclonal antibodies, polyclonal antibodies, multi-specific antibodies (e.g., bispecific or bifunctional antibodies), and antibody fragments so long as they exhibit the desired antigen-binding activity.

The term “antibody fragment” as used herein refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g., scFv), and single-domain antibodies.

The term “Fab fragment” as used herein refers to an immunoglobulin fragment comprising a VL domain and a constant domain of a light chain (CL), and a VH domain and a first constant domain (CH1) of a heavy chain.

The terms “variable region” or “variable domain” as used herein refers to the domain of an immunoglobulin or antibody heavy or light chain that is generally involved in binding the immunoglobulin or antibody to antigen. The variable domains of the heavy chain and light chain (VH and VL, respectively) of an immunoglobulin or antibody generally have similar structures, with each domain comprising four conserved framework regions (FRs) and three Complementarity-determining regions (CDRs).

The term “complementarity determining regions” or “CDRs” contain the antigen-contacting residues (“antigen contacts”). Generally, antibodies comprise six CDRs: three in the VH (CDR-H1, CDR-H2, CDR-H3), and three in the VL (CDR-L1, CDR-L2, CDR-L3). CDRs occurring at amino acid residues 24-34 (CDR-L1), 50-56 (CDR-L2), 89-97 (CDR-L3), 31-35b (CDR-H1), 50-65 (CDR-H2), and 95-102 (CDR-H3) (Kabat et al., Sequences of Proteins of Immunological Interest, 5th Ed. Public Health Service, National Institutes of Health, Bethesda, MD (1991)). Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs)

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649, U.S. Pat. Nos. 4,946,778 and 5,260,203, the disclosures of which are incorporated by reference.

A “human immunoglobulin” as used herein is one which possesses an amino acid sequence which corresponds to that of an immunoglobulin produced by a human or a human cell or derived from a non-human source that utilizes human immunoglobulin repertoires or other human immunoglobulin-encoding sequences. This definition of a human immunoglobulin specifically excludes a humanized immunoglobulin comprising non-human antigen-binding residues.

The term “humanized antibody” as used herein refers to an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework, and such substitutions are herein referred to as back-mutations. Humanized or other monoclonal antibodies can have additional conservative amino acid substitutions which have substantially no effect on antigen binding or other immunoglobulin functions.

The term “Fc domain” or “Fc region” as used herein is used to define a C-terminal region of an immunoglobulin heavy chain that contains at least a portion of the constant region. The term includes native sequence Fc regions and variant Fc regions. An IgG Fc region comprises an IgG CH2 and an IgG CH3 domain. The CH3 region herein may be a native sequence CH3 domain or a variant CH3 domain (e.g., a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical immunoglobulin heavy chains as herein described. Unless otherwise specified herein, numbering of amino acid residues in the Fc region or constant region is according to the EU numbering system.

The term “effector functions” as used herein refers to those biological activities attributable to the Fc region of an immunoglobulin, which vary with the immunoglobulin isotype. Examples of immunoglobulin effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, “specific binding” is meant that the binding is selective for the antigen and can be discriminated from unwanted or non-specific interactions. The ability of an immunoglobulin to bind to a specific antigen can be measured either through an enzyme-linked immunosorbent assay (ELISA) or other techniques familiar to one of skill in the art, e.g., surface plasmon resonance (SPR) technique.

The terms “affinity” or “binding affinity” as used herein refers to the strength of the sum total of non-covalent interactions between a single binding site of a molecule (e.g., an antibody) and its binding partner (e.g., an antigen). The affinity of a molecule X for its partner Y can generally be represented by the dissociation constant (KD), which is the ratio of dissociation and association rate constants (koff and kon, respectively). A particular method for measuring affinity is SPR.

The term “immunogenicity” as used herein refers to the ability of an antibody or antigen binding fragment to elicit an immune response (humoral or cellular) when administered to a recipient and includes, for example, the human anti-mouse antibody (HAMA) response. A HAMA response is initiated when T-cells from a subject make an immune response to the administered antibody. The T-cells then recruit B-cells to generate specific “anti-antibody” antibodies.

The term “immune cell” as used herein means any cell of hematopoietic lineage involved in regulating an immune response against an antigen (e.g., an autoantigen). In various embodiments, an immune cell is, e.g., a T cell, a B cell, a dendritic cell, a monocyte, a natural killer cell, a macrophage, Langerhan's cells, or Kuffer cells.

The term “reduced binding”, as used herein refers to a decrease in affinity for the respective interaction, as measured for example by SPR. Conversely, “increased binding” refers to an increase in binding affinity for the respective interaction.

The term “polymer” as used herein generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions.

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The terms “label” or “labeled” as used herein refers to the incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In various embodiments, labels are attached by spacer arms of various lengths to reduce potential steric hindrance.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

It is understood that aspect and embodiments of the disclosure described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variations.

### PD1 Blocking Antibody

In one aspect, the PD1 blocking antibody guides IL-15 moiety of the VitoKine to the TILs in the tumor microenvironment (TME) and restrict the activation of the VitoKine locally to improve the therapeutic index. In another aspect, the PD1 blocking antibody guides the IL-15 moiety of the immunocytokine to the TILs in the TME. In various embodiments, the PD1 blocking antibodies were optimized through modifications in the variable domains of pembrolizumab by germline sequence substitutions of the CDR residues, germline sequence substitutions of the framework residues, and adoption of the most prevalent and better behaving VH3 human germline family sequence as the acceptor framework. In various embodiments, these modifications were implemented individually or in combination to develop optimized PD1 blocking antibodies. In various embodiments, these optimized PD1 blocking antibodies exhibit a high binding affinity to PD1, function to inhibit PD1 with equal or comparable potency as pembrolizumab, have a higher sequence similarity score to its closest human germline sequence, resulting in an improved degree of humanness compared to pembrolizumab, and are predicted to have lower hydrophobicity, leading to a reduced aggregation propensity than pembrolizumab. In various embodiments, PD1 Ab-IL-15 VitoKine constructs and PD1-targeted IL-15 immunocytokines based on these optimized PD1 blocking antibodies are predicted to have enhanced developability properties. In various embodiments, the PD1 antibody comprises a light chain variable region with the sequence selected from the group of sequences set forth in SEQ ID NOS: 3-5, and a heavy chain variable region with the sequence selected from the group of sequences set forth in SEQ ID NOS: 7-18. In various embodiments, the PD1 antibody comprises a light chain sequence set forth in SEQ ID NO: 44, and a heavy chain with the sequence selected from the group of sequences set forth in SEQ ID NOS: 45-49.

### IL-15 Domain

Interleukin-15 (IL-15) is a cytokine identified by two independent groups based upon its ability to stimulate proliferation of the IL-2-dependent CTLL-2 T-cell line in the presence of neutralizing anti-IL-2 antibodies (Steel et al., Trends Pharm Sci, 33:35-41, 2012). IL-15 and IL-2 have similar biologic properties in vitro, consistent with their shared receptor (R) signaling components (IL-15Rβγc). However, specificity for IL-15 versus IL-2 is provided by unique private α-chain receptors that complete the IL-15Rαβγ and IL-2Rαβγ heterotrimeric high-affinity receptor complexes and thereby allow differential responsiveness depending on the ligand and high-affinity receptor expressed. Intriguingly, both IL-15 and IL-15Rα transcripts have a much broader tissue distribution than IL-2/IL-2Rα. Further, multiple complex posttranscriptional regulatory mechanisms tightly control IL-15 expression. Thus, based upon complex regulation, as well as differential patterns of IL-15 and IL-15Rα expression, it is likely that the critical in vivo functions of this receptor/ligand pair differ from those of IL-2 and IL-2Rα. Studies to date examining the biology of IL-15 have identified several key nonredundant roles, such as IL-15's importance during natural killer (NK) cell, NK-T cell, and intestinal intraepithelial lymphocyte development and function. The role for IL-15 during autoimmune processes such as rheumatoid arthritis and malignancies such as adult T-cell leukemia suggest that dysregulation of IL-15 may result in deleterious effects for the host (Fehniger et al., Blood, 97:14-32, 2001).

As used herein, the terms “native IL-15” and “native interleukin-15” in the context of proteins or polypeptides refer to any naturally occurring mammalian interleukin-15 amino acid sequences, including immature or precursor and mature forms. Non-limiting examples of GenBank Accession Nos. for the amino acid sequence of various species of native mammalian interleukin-15 include NP_032383 (Mus musculus, immature form), AAB60398 (Macaca mulatta, immature form), NP_000576 (human, immature form), CAA62616 (human, immature form), AAI00964 (human, immature form), and AAH18149 (human). In various embodiments of the present invention, native IL-15 is the immature or precursor form of a naturally occurring mammalian IL-15. In other embodiments, native IL-15 is the mature form of a naturally occurring mammalian IL-15. In various embodiments, native IL-15 is the precursor form of naturally occurring human IL-15. In various embodiments, native IL-15 is the mature form of naturally occurring human IL-15. In various embodiments, the IL-15 in the VitoKine and immunocytokine constructs of the present invention is derived from the amino acid sequence of the human IL-15 mature sequence set forth in SEQ ID NO: 116:

(SEQ ID NO: 116)
NWVNVISDLKKIEDLIQSMHIDATLYTESDVHPSCKVTAMKCFLL
ELQVISLESGDASIHDTVENLIILANNSLSSNGNVTESGCKECEE
LEEKNIKEFLQSFVHIVQMFINTS

In various embodiments, the IL-15 domain will be an IL-15 variant (or mutant) comprising a sequence derived from the sequence of the mature human IL-15 polypeptide as set forth in SEQ ID NO: 116. In various embodiments, the IL-15 variant comprises a different amino acid sequence than the native (or wild type) IL-15 protein. In various embodiments, the IL-15 variant binds to the IL-15Rα polypeptide and functions as an IL-15 agonist or antagonist. In various embodiments, the IL-15 variant can function as an IL-15 agonist or antagonist independent of its association with IL-15Rα. IL-15 agonists are exemplified by comparable or increased biological activity compared to wild type IL-15. IL-15 antagonists are exemplified by decreased biological activity compared to wild type IL-15 or by the ability to inhibit IL-15-mediated responses. In various embodiments, the IL-15 variant binds with increased or decreased activity to the IL-15Rβγc receptors. In various embodiments, the sequence of the IL-15 variant has at least one amino acid change, e.g., substitution or deletion, compared to the native IL-15 sequence, such changes resulting in IL-15 agonist or antagonist activity. In various embodiments, the amino acid substitutions/deletions are in the domains of IL-15 that interact with IL-15Rβ and/or γc. In various embodiments, the amino acid substitutions/deletions do not affect binding to the IL-15Rα polypeptide or the ability to produce the IL-15 variant. Suitable amino acid substitutions/deletions to generate IL-15 variants can be identified based on known IL-15 structures, comparisons of IL-15 with homologous molecules such as IL-15 with known structure, through rational or random mutagenesis and functional assays, as provided herein, or other empirical methods. Additionally, suitable amino acid substitutions can be conservative or non-conservative changes and insertions of additional amino acids. In various embodiments, the amino acid change is one or more amino acid substitutions at position 30, 32, 63, 68, 108, 109 or 112 of SEQ ID NO: 116. In various embodiments, the amino acid change is the substitution of D to T at position 30, H to D or E or N or Q at position 32, V to F or A or K or R at position 63, I to F or H or D or K or Q or G at position 68, Q to A or D or E or F or H or K or L or M or N or S or T or Y at position 108, M to A or H or R at position 109, N to D or G or P or R at position 112 of the mature human IL-15 sequence, or any combination of these substitutions. In various embodiments, the amino acid change is 1, or 2, or 3, or 4 amino acid deletion at the N-terminus of SEQ ID NO: 116. In various embodiments, the amino acid change is 1, or 2, or 3, or 4, or 5, or 6, or 7, or 8, or 9, or 10 amino acid deletion at the C-terminus of SEQ ID NO: 116. In various embodiments, the IL-15 domain has any combinations of amino acid substitutions, deletions and insertions. In various embodiments, IL-15 variant is selected from the group of sequences set forth in SEQ ID NOS: 117-163.

### IL-15Rα Domain

IL-15 receptor is a type I cytokine receptor consisting of a beta (β) and gamma (γ) subunit that it shares with IL-2 receptor, and an alpha (α) subunit which binds IL-15 with a high affinity. The full-length human IL-15Rα is a type-1 transmembrane protein with a signal peptide of 32 AAs, an extracellular domain of 173 AAs, a transmembrane domain of 21 AAs, a 37-AA cytoplasmic tail, and multiple N- or O-linked glycosylation sites (Anderson et al., J. Biol Chem, 270:29862-29869, 1995). It has been previously demonstrated that a natural soluble form of IL-15R alpha chain corresponding to the entire extracellular domain of IL-15R alpha behaves as a high affinity IL-15 antagonist. However, in sharp contrast with that finding, it was demonstrated that a recombinant, soluble sushi domain of IL-15R alpha, which bears most of the binding affinity for IL-15, behaves as a potent IL-15 agonist by enhancing its binding and biological effects (proliferation and protection from apoptosis) through the IL-15R beta/gamma heterodimer, whereas it does not affect IL-15 binding and function of the tripartite IL-15R alpha/beta/gamma membrane receptor. These results suggested that, if naturally produced, such soluble sushi domains might be involved in the IL-15 transpresentation mechanism (Mortier et al., J. Biol Chem, 281:1612-1619, 2006).

As used herein, the terms “native IL-15Rα” and “native interleukin-15 receptor alpha” in the context of proteins or polypeptides refer to any naturally occurring mammalian interleukin-15 receptor alpha (“IL-15Rα”) amino acid sequence, including immature or precursor and mature forms and naturally occurring isoforms. Non-limiting examples of GenBank Accession Nos. for the amino acid sequence of various native mammalian IL-15Rα include NP_002180 (human), ABK41438 (Macaca mulatta), NP_032384 (Mus musculus), Q60819 (Mus musculus), CA141082 (human). In various embodiments, native IL-15Rα is the full-length form of a naturally occurring mammalian IL-15Rα polypeptide. In various embodiments, native IL-15Rα is the immature form of a naturally occurring human IL-15Rα polypeptide. In various embodiments, native IL-15Rα is the mature form of a naturally occurring human IL-15Rα polypeptide. In various embodiments, native IL-15Rα is the full-length form of a naturally occurring human IL-15Rα polypeptide. In various embodiments, a native IL-15Rα protein or polypeptide is isolated or purified. In various embodiments, the IL-15Rα domain is derived from the amino acid sequence of the human IL-15Rα sequence set forth in SEQ ID NO: 164:

(SEQ ID NO: 164)
MAPRRARGCRTLGLPALLLLLLLRPPATRGITCPPPMSVEHADIW
VKSYSLYSRERYICNSGFKRKAGTSSLTECVLNKATNVAHWTTPS
LKCIRDPALVHQRPAPPSTVTTAGVTPQPESLSPSGKEPAASSPS
SNNTAATTAAIVPGSQLMPSKSPSTGTTEISSHESSHGTPSQTTA
KNWELTASASHQPPGVYPQGHSDTTVAISTSTVLLCGLSAVSLLA
CYLKSRQTPPLASVEMEAMEALPVTWGTSSRDEDLENCSHHL

In various embodiments, the functional IL-15Rα domain is the IL-15RαSushi+ domain comprising the amino acid sequence set forth in SEQ ID NO: 165:

(SEQ ID NO: 165)
ITCPPPMSVEHADIWVKSYSLYSRERYICNSGFKRKAGTSSLTEC
VLNKATNVAHWTTPSLKCIRDPALVHQRPAPP

In one aspect, PD1 Ab-IL-15 VitoKine constructs of the present invention contain a covalently linked IL-15Rα as the concealing moiety domain. In various embodiments, the concealing moiety domain is an IL-15Rα extracellular domain or a functional fragment thereof. In various embodiments, the concealing moiety domain is an IL-15RαSushi+ domain comprising the amino acid sequence as set forth in SEQ ID NO: 165. In various preferred embodiments, the concealing moiety domain is a variant of IL-15RαSushi+ domain. The concealing moiety domain is mainly used to reversibly conceal the activity of the IL-15 domain in the VitoKine construct.

In various embodiments, IL-15RαSushi+ (SEQ ID NO: 165), the truncated cognate co-receptor of IL-15 that recapitulate the majority of binding affinity of the full-length IL-15Rα (SEQ ID NO: 164), was covalently linked to the IL-15 domain in a PD1 Ab-IL-15 VitoKine to conceal the activity of IL-15. In various embodiments, the effectiveness of IL-15 activity concealing can be further adjusted by fine-tuning the length and composition of the linker connecting the IL-15 and IL-15RαSushi+ domains. As can be appreciated by skilled artisan, the concealing domain (D3) can vary from the sequence set forth in SEQ ID NO: 164 as far as it can recapitulate the majority of binding activity of the full-length IL-15Rα (SEQ ID NO: 164), namely being a functional fragment. The distinctness of IL-15 VitoKine design depending on the full use of the unique features of IL-15 pathway, including the exceptionally high affinity between IL-15 and IL-15α (30 pM), and that the complexation of IL-15 enhance the activity of IL-15 in vivo. After the cleavage of the linker connecting the IL-15 and IL-15αSushi+ by proteases that are upregulated at disease site, IL-15RαShushi+ or any function fragment derived from IL-15Rα is expected to remain non-covalently associated with IL-15 and to augment IL-15 activity.

In another aspect, the PD1 targeted IL-15 immunocytokine of the present invention comprises a non-covalently associated IL-15RαSushi+ domain. The non-covalent complexation of IL-15RαSushi+ with IL-15 was achieved in cell culture co-expression due to the exceptionally high affinity between IL-15 and IL-15α (30 pM). As disclosed in WO2019246379 by the current inventors, covalent complexation of IL-15α with IL-15 significantly enhances the developability profiles of the corresponding fusion proteins.

### L1 and L2 Linkers in PD1 Ab-IL-15 VitoKine

### Cleavable Linkers

A cleavable linker, or a linker susceptible to a disease-associated enzyme may contain a moiety, e.g., a protein substrate, capable of being specifically cleaved by a protease that is present at elevated levels at the disease site as compared to non-disease tissues. Literature contains multiple reports on increased levels of enzymes with known substrates in various types of cancers, e.g., solid tumors. See, e.g., La Rocca et al., Brit. J. Cancer 90:1414-1421 and Ducry et al., Bioconjug. Chem. 21:5-13, 2010, each of which is incorporated by reference herein in its entirety. In various embodiments, the protease capable of cleaving a protease-cleavable linker is selected from the group consisting of metalloproteinase, e.g., matrix metalloproteinase (MMP) 1-28, serine protease, e.g., urokinase-type plasminogen activator (uPA) and matriptase, cysteine protease, e.g., legumain, aspartic protease, and cathepsin protease. Exemplary proteases are provided in Table 2:

TABLE 2

| Protease family | Protease | RefSeq (protein) |
|---|---|---|
| Matrix | MMP-1 (Collagenase 1) | NP_002412 |
| Metalloproteins | MMP-2 (Gelatinase A) | NP_001121363 |
| (MMPs) | MMP-3 (Stromelysin 1) | NP_002413 |
| | MMP-7 (Matrilysin 1) | NP_002414 |
| | MMP-8 (Collagenase 2) | NP_002415 |
| | MMP-9 (Gelatinase B) | NP_004985 |
| | MMP-10 (Stromelysin 2) | NP_002416 |
| | MMP-11 (Stromelysin 3) | NP_005931.2 |
| | MMP-12 (Macrophage | NP_002417.2 |
| | Elastase) | |
| | MMP-13 (Collagenase 3) | NP_002418 |
| | MMP-14 (MT1-MMP) | NP_004986 |
| | MMP-15 (MT2-MMP) | NP_002419 |
| | MMP-19 | NP_002420 |
| | MMP-23 (CA-MMP) | NP_008914 |
| | MMP-24 (MT5-MMP) | NP_006681 |
| | MMP-26 (Matrilysin 2) | NP_068573.2 |
| | MMP-27 (CMMP) | NP_071405.2 |
| Cysteine Proteases | Legumain | NP_001008530 |
| | Cathepsin C | NP_001805.3 |
| | Cathepsin K | NP_000387 |
| | Cathepsin L1 | NP_001903 |
| | Cathepsin S | NP_004070 |
| | Cathepsin X (Cathepsin Z) | NP_001327.2 |
| Aspartase Proteases | Cathepsin D | NP_001900 |
| | Cathepsin E | NP_001901 |
| | Secretase (BACE1) | NP_001193978 |
| Serine Proteases | Urokinase plasminogen | NM_002658 |
| | activator (uPA) | |
| | Tissue-type plasminogen | NP_000921 |
| | activator (tPA) | |
| | Plasmin | NP_000292 |
| | Thrombin | NP_000497 |
| | Prostate-specific antigen | NP_001639 |
| | (PSA, KLK3) | |
| | human neutrophil elastase | NP_001963 |
| | (HNE) | |
| | Elastase (CELA1) | NP_001962.3 |
| | Tryptase | NP_003285.2 |
| | Matriptase (ST14) | NP_068813 |
| Disintegrin and | ADAM-10 | NP_001101 |
| metalloproteinase | ADAM-17 | NP_003174 |
| (ADAM) | | |

Exemplary protease substrate peptide sequences, which can be used as protease cleavable linkers with or without peptide spacers, are provided in Table 3:

TABLE 3

| | | SEQ |
|---|---|---|
| | Substrate | ID |
| Proteases | peptide | NO: |
| MMP-2, 7, 9, 14 | SPLGLAGS | 54 |
| MMP-2, 7, 9, 14, matriptase | EPLELRAG | 55 |
| Matriptase, uPA, Legumain | LSGRSDNH | 56 |
| MMP-2 | GPLGIAGQ | 57 |
| MMP-2, 14 | GTAHLMGG | 58 |
| MMP-14 | RIGSLRTA | 59 |
| MMP-14 | SGRSENIRTA | 60 |
| MMP-2, 9 | GPLGMLSQ | 61 |
| MMP-2, 9 | GPAGMKGL | 62 |
| MMP-9, uPA | RPSASRSA | 63 |
| MMP | PLGLAG | 64 |
| uPA | LGGSGRSANAILE | 65 |
| uPA | GGSGRSANAI | 66 |
| uPA | SGRSA | 67 |
| Legumain | AANL | 68 |
| Legumain | GPTNKVR | 69 |
| Cathepsin C | GFFY | 70 |
| Cathepsin D | GPICFRLG | 71 |
| Cathepsin E | RQAGFSL | 72 |
| Matriptase | RQARAVGG | 73 |
| Matriptase | PMAKK | 74 |
| Prostate Specific antigen | HSSKLQ | 75 |
| MMP-2, 9 and Matriptase | GPLGMLSQPMAKK | 76 |
| Matriptase and MMP-2,9 | PMAKKGPLGMLSQ | 77 |

In various embodiments, the protease is MMP-9 or MMP-2. In a further specific embodiment, the protease is matriptase. In a further specific embodiment, the protease is MMP-14. In further specific embodiment, the protease is legumain. In various embodiments, the protease cleavable linker may contain two or more protease substrate sequences. In various embodiments, the proteases are MMP-2/MMP-9 and matriptase. In various embodiments, the protease-cleavable linker comprises the protease recognition sequence ‘GPLGMLSQ’ (SEQ ID NO: 61). In various embodiments, the protease-cleavable linker comprises the protease recognition sequence ‘SGRSENIRTA’ (SEQ ID NO: 60). In various embodiments, the protease-cleavable linker comprises the protease recognition sequence ‘GPTNKVR’ (SEQ ID NO: 69). In various embodiments, the protease-cleavable linker comprises the protease recognition sequence ‘PMAKK’ (SEQ ID NO: 74). In various embodiments, the protease-cleavable linker comprises the protease recognition sequence ‘GPLGMLSQPMAKK’ (SEQ ID NO: 76). In various embodiments, the protease-cleavable linker comprises the protease recognition sequence ‘PMAKKGPLGMLSQ’ (SEQ ID NO: 77).

In various embodiments, peptide spacers may be incorporated on either side of a protease cleavable sequence or to flank both sides of a protease cleavable sequence, or as a non-cleavable linker without a protease substrate site. Peptide spacer serves to position a cleavable linker, making it more readily \] accessible to the enzyme responsible for cleavage. The length and composition of a peptide spacer can be fine-tuned to balance the accessibility for enzymatic cleavage and the spatial constraint required to reversibly conceal the D2 domain from exerting its biological activity. A peptide spacer may include 1-100 amino acids. Suitable peptide spacers are known in the art, which include, but are not limited to, peptide linkers containing flexible amino acid residues, such as glycine and serine. In various embodiments, a peptide spacer can contain 1 to 12 amino acids including motifs of G, S, GSGG (SEQ ID NO: 104), GGSS (SEQ ID NO: 105), GSGS (SEQ ID NO: 109), GSGSGS (SEQ ID NO: 110), GSGSGSGS (SEQ ID NO: 111), GSGSGSGSGS (SEQ ID NO: 112), or GSGSGSGSGSGS (SEQ ID NO: 113). In other embodiments, a peptide spacer can contain motifs of (GGGGS) (SEQ ID NO: 106)ₙ, wherein n is an integer from 1 to 10. In other embodiments, a peptide spacer can also contain amino acids other than glycine and serine. A peptide spacer is stable under physiological conditions as well as at a diseased site, such as a cancer site.

Exemplary protease cleavable linkers with peptide spacers flanking the protease substrate peptides (underscored) are provided in Table 4:

TABLE 4

| | SEQ |
|---|---|
| | ID |
| Protease cleavable linker | NO: |
| GGGSGGGGSGGGGSLSGRSDNHGGSGGGGS | 78 |
| GSSSGRSENIRTAGT | 79 |
| GGGGSGGGGSGGGSLGGSGRSANAILEGGSGGGGS | 80 |
| GGGGSGGGGSLGGSGRSANAILEGGGGS | 81 |
| GGGGSLGGSGRSANAILEGGS | 82 |
| GGGSGPTNKVRGGS | 83 |
| GGSGPLGMLSQGGGS | 84 |
| GGPLGMLSQS | 85 |
| GGGPLGMLSQGGS | 86 |
| GGPTNKVRGS | 87 |
| GRQARAVGGS | 88 |
| GGGSGRSENIRTAGG | 89 |
| SGGPGPAGMKGLPGS | 90 |
| GGGGSPMAKKGGGGS | 91 |
| GGPLGMLSQPMAKKS | 92 |
| GGSGPLGMLSQPMAKKGGGS | 93 |
| GGGPMAKKGPLGMLSQGGGS | 94 |

### Non-Cleavable Linkers

Non-cleavable linker provides covalent linkage and additional structural and/or spatial flexibility between protein domains. As is known in the art, peptide linkers containing flexible amino acid residues, such as glycine and serine, can be used as non-cleavable linkers. In various embodiments, non-cleavable linker may include 1-100 amino acids. In various embodiments, a spacer can contain motifs of GS (SEQ ID NO: 116), GGS (SEQ ID NO: 117), GGGGS (SEQ ID NO: 118), GGSG (SEQ ID NO: 119), or SGGG (SEQ ID NO: 120). In other embodiments, a linker can contain motifs of (GGGGS) (SEQ ID NO: 118) n, wherein n is an integer from 1 to 10. In other embodiments, a linker can also contain amino acids other than glycine and serine. In another embodiment, the non-cleavable linker can be a simple chemical bond, e.g., an amide bond (e.g., by chemical conjugation of PEG). A non-cleavable linker is stable under physiological conditions as well as at a diseased site, such as a cancer site. Exemplary non-cleavable linkers are provided in Table 5:

TABLE 5

| | SEQ ID |
|---|---|
| Linker sequence | NO: |
| EPKSSDKTHTSPPS | 95 |
| GGGSGGGSGGGS | 96 |
| GGGS | 97 |
| GSSGGSGGS | 98 |
| GSSGT | 99 |
| GGGGSGGGGSGGGS | 100 |
| AEAAAKEAAAKEAAAKA | 101 |
| GGGGSGGGGSGGGGSGGGGS | 102 |
| GGGSGGGS | 103 |
| GSGG | 104 |
| GGSS | 105 |
| GGGGS | 106 |
| GGSGG | 107 |
| SGGG | 108 |
| GSGS | 109 |
| GSGSGS | 110 |
| GSGSGSGS | 111 |
| GSGSGSGSGS | 112 |
| GSGSGSGSGSGS | 113 |
| GGGGSGGGGS | 114 |
| GGGGSGGGGSGGGGS | 115 |

### A Combination of Cleavable and Non-Cleavable Linkers

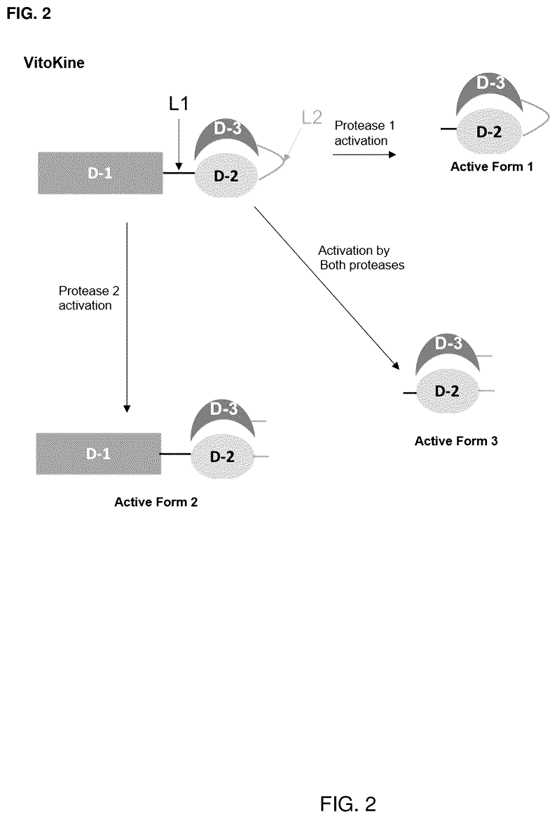

In various embodiments, the L1 and L2 linkers can be both cleavable or a combination of cleavable and non-cleavable linkers to yield different forms of active moiety of the IL-15 domain to fulfill specific therapeutic objectives, optimize the risk to benefit ratio, or align with diverse properties of the cytokine. The exemplary active forms released by cleavage of the linkers are depicted in FIG. 2. The active form 1 derived from cleavage of the L1 linker and the active form 3 derived from cleavage of L1 and L2 likers, are both short-acting cytokines due to their release from the targeting antibody after proteolysis. These two active forms contains either covalently linked IL-15RαSushi+ domain or non-covalently complexed IL-15RαSushi+ domain and display distinct activity in the local environment. After acting locally, the short-acting active forms can be eliminated from systemic circulation quickly, leading to reduced toxicities. In contrast, the active form 2 derived from the cleavage of the L2 linker is a functionally fully restored IL-15 fused to the PD1 Ab at or near the disease site. This active form is capable of cis-activating IL-15R signaling on PD1-expressing T cell at or near the disease site, which synergistically enhances the two pathways and boosts the anticancer immune response, while minimizing systematic toxicity.

### Polynucleotides

In another aspect, the present disclosure provides isolated nucleic acid molecules comprising a polynucleotide IL-15, an IL-15 variant, IL-15Rα, an PD1 blocking antibody, an antibody fragment, or a PD1 targeted IL-15 immunocytokine, or a PD1 Ab-IL-15 VitoKine construct of the present disclosure. The subject nucleic acids may be single-stranded or double stranded. Such nucleic acids may be DNA or RNA molecules. DNA includes, for example, cDNA, genomic DNA, synthetic DNA, DNA amplified by PCR, and combinations thereof. Synthetic DNA is available from chemical synthesis of overlapping oligonucleotide fragments followed by assembly of the fragments to reconstitute part or all of the coding regions and flanking sequences. RNA may be obtained from prokaryotic expression vectors which direct high-level synthesis of mRNA, such as vectors using T7 promoters and RNA polymerase. The DNA molecules of the disclosure include full-length genes as well as polynucleotides and fragments thereof. The full-length gene may also include sequences encoding the N-terminal signal sequence. Such nucleic acids may be used, for example, in methods for making the novel VitoKine constructs.

In various embodiments, the isolated nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein.

In various embodiments, the recombinant nucleic acids of the present disclosure may be operably linked to one or more regulatory nucleotide sequences in an expression construct. Regulatory sequences are art-recognized and are selected to direct expression of the VitoKine construct. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the present disclosure. The promoters may be either naturally occurring promoters, or hybrid promoters that combine elements of more than one promoter. An expression construct may be present in a cell on an episome, such as a plasmid, or the expression construct may be inserted in a chromosome. In various embodiments, the expression vector contains a selectable marker gene to allow the selection of transformed host cells. Selectable marker genes are well known in the art and will vary with the host cell used.

In another aspect of the present disclosure, the subject nucleic acid is provided in an expression vector comprising a nucleotide sequence encoding the pharmaceutical compositions of the invention and operably linked to at least one regulatory sequence. The term “expression vector” refers to a plasmid, phage, virus or vector for expressing a polypeptide from a polynucleotide sequence. Vectors suitable for expression in host cells are readily available and the nucleic acid molecules are inserted into the vectors using standard recombinant DNA techniques. Such vectors can include a wide variety of expression control sequences that control the expression of a DNA sequence when operatively linked to it may be used in these vectors to express DNA sequences encoding a PD1 targeted IL-15 immunocytokine construct or a PD1 Ab-IL-15 VitoKine construct. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered. An exemplary expression vector suitable for expression of the pharmaceutical compositions of the invention is the pDSRa, and its derivatives, containing polynucleotides coding for the pharmaceutical compositions of the invention, as well as any additional suitable vectors known in the art or described below.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of an immunocytokine or a VitoKine construct include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In various embodiments, a vector will be designed for production of the subject construct in Chinese Hamster Ovary (CHO) cells or Human Embryonic Kidney 293 (HEK293) cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject constructs in cells propagated in culture, e.g., to produce proteins, including fusion proteins or variant proteins, for purification.

This present disclosure also pertains to a host cell transfected with a recombinant gene including a nucleotide sequence coding an amino acid sequence for one or more of the subject constructs. The host cell may be any prokaryotic or eukaryotic cell. For example, a construct of the present disclosure may be expressed in bacterial cells such as E. coli, insect cells (e.g., using a baculovirus expression system), yeast, or mammalian cells. Other suitable host cells are known to those skilled in the art, such as CHO cells, or HEK293 cells.

Accordingly, the present disclosure further pertains to methods of producing the subject constructs. For example, a host cell transfected with an expression vector encoding an immunocytokine construct or a VitoKine construct can be cultured under appropriate conditions to allow expression of the VitoKine construct to occur. The construct may be secreted and isolated from a mixture of cells and medium containing the VitoKine construct. Alternatively, the construct may be retained cytoplasmically or in a membrane fraction and the cells harvested, lysed and the protein isolated. A cell culture includes host cells, media and other byproducts. Suitable medias for cell culture are well known in the art.

The polypeptides and proteins of the present disclosure can be purified according to protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “isolated polypeptide” or “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography; isoelectric focusing; gel electrophoresis; and combinations of these techniques. As is generally known in the art, it is believed that the order of conducting the various purification steps may be changed, or that certain steps may be omitted, and still result in a suitable method for the preparation of a substantially purified polypeptide.

### Pharmaceutical Compositions

In another aspect, the present disclosure provides a pharmaceutical composition comprising an immunocytokine construct or a VitoKine construct in admixture with a pharmaceutically acceptable carrier. Such pharmaceutically acceptable carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrin); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

When parenteral administration is contemplated, the therapeutic pharmaceutical compositions may be in the form of a pyrogen-free, parenterally acceptable aqueous solution comprising the desired polypeptide construct in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions.

In various embodiments, the therapeutic pharmaceutical compositions may be formulated for targeted delivery using a colloidal dispersion system. Colloidal dispersion systems include macromolecule complexes, nanocapsules, microspheres, beads, and lipid-based systems including oil-in-water emulsions, micelles, mixed micelles, and liposomes. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

In various embodiments, oral administration of the pharmaceutical compositions is contemplated. Pharmaceutical compositions that are administered in this fashion can be formulated with or without those carriers customarily used in the compounding of solid dosage forms such as tablets and capsules. In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

In various embodiments, topical administration of the pharmaceutical compositions, either to skin or to mucosal membranes, is contemplated. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the disclosure (e.g., a VitoKine construct), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Additional pharmaceutical compositions contemplated for use herein include formulations involving polypeptides in sustained- or controlled-delivery formulations. In various embodiments, pharmaceutical compositions may be formulated in nanoparticles, as slow-release hydrogel, or incorporated into oncolytic viruses. Such nanoparticles methods include, e.g., encapsulation in nanoparticles composed of polymers with a hydrophobic backbone and hydrophilic branches as drug carriers, encapsulation in microparticles, insertion into liposomes in emulsions, and conjugation to other molecules. Examples of nanoparticles include mucoadhesive nanoparticles coated with chitosan and Carbopol (Takeuchi et al., Adv. Drug Deliv. Rev. 47(1): 39-54, 2001) and nanoparticles containing charged combination polyesters, poly(2-sulfobutyl-vinyl alcohol) and poly(D,L-lactic-co-glycolic acid) (Jung et al., Eur. J. Pharm. Biopharm. 50(1): 147-160, 2000). Albumin-based nanoparticle compositions have been developed as a drug delivery system for delivering hydrophobic drugs such as a taxane. See, for example, U.S. Pat. Nos. 5,916,596; 6,506,405; 6,749,868; 6,537,579; 7,820,788; and 7,923,536. Abraxane®, an albumin stabilized nanoparticle formulation of paclitaxel, was approved in the United States in 2005 and subsequently in various other countries for treating metastatic breast cancer.

Techniques for formulating a variety of other sustained- or controlled-delivery means, such as liposome carriers, bio-erodible microparticles or porous beads and depot injections, are also known to those skilled in the art.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.0001 mg/kg to up to about 100 mg/kg or more, depending on the factors mentioned above. Polypeptide compositions may be preferably injected or administered intravenously. Long-acting pharmaceutical compositions may be administered every three to four days, every week, biweekly, triweekly, monthly, or even longer durations depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intratumoral, intracerebral (intra-parenchymal), intracerebroventricular, intramuscular, intra-ocular, intraarterial, intraportal, intralesional routes, intramedullary, intrathecal, intraventricular, intravesical, transdermal, subcutaneous, or intraperitoneal; as well as intranasal, enteral, topical, sublingual, urethral, vaginal, or rectal means, by sustained release systems or by implantation devices. Where desired, the compositions may be administered by bolus injection or continuously by infusion, or by implantation device. Alternatively, or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

### Therapeutic Uses

The present disclosure provides for a method of treating cancer cells in a subject, comprising administering to said subject a therapeutically effective amount (either as monotherapy or in a combination therapy regimen) of a pharmaceutical composition of the present disclosure in pharmaceutically acceptable carrier, wherein such administration inhibits the growth and/or proliferation of a cancer cell. Specifically, an immunocytokine or a VitoKine construct of the present disclosure is useful in treating disorders characterized as cancer. Such disorders include, but are not limited to solid tumors, such as cancers of the breast, respiratory tract, brain, reproductive organs, digestive tract, urinary tract, eye, liver, skin, head and neck, thyroid, parathyroid and their distant metastases, lymphomas, sarcomas, multiple myeloma and leukemia. Examples of breast cancer include, but are not limited to invasive ductal carcinoma, invasive lobular carcinoma, ductal carcinoma in situ, and lobular carcinoma in situ. Examples of cancers of the respiratory tract include but are not limited to small-cell and non-small-cell lung carcinoma, as well as bronchial adenoma and pleuropulmonary blastoma. Examples of brain cancers include but are not limited to brain stem and hypothalamic glioma, cerebellar and cerebral astrocytoma, neuroblastoma, medulloblastoma, ependymoma, as well as neuroectodermal and pineal tumor. Tumors of the male reproductive organs include but are not limited to prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, liver, breast, pancreatic, rectal, small-intestine, and salivary gland cancers. Tumors of the urinary tract include, but are not limited to bladder, penile, kidney, renal pelvis, ureter, and urethral cancers. Eye cancers include but are not limited to intraocular melanoma and retinoblastoma. Examples of liver cancers include but are not limited to hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to nasopharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include but are not limited to sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

In various embodiments, the pharmaceutical composition of the present disclosure can be used as a single agent for treatment of all kinds of cancers, including but not limited to non-small cell lung, small cell lung, melanoma, renal cell carcinoma, urothelial, liver, breast, pancreatic, colorectal, gastric, prostate, and sarcoma.

Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an IC₅₀ (half maximal inhibitory concentration). A dose can then be formulated in animal models to achieve a circulating plasma concentration range that includes the IC₅₀ as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, and the route of administration. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

An exemplary, non-limiting daily dosing range for a therapeutically or prophylactically effective amount of a composition of the disclosure can be 0.0001 to 100 mg/kg, 0.0001 to 90 mg/kg, 0.0001 to 80 mg/kg, 0.0001 to 70 mg/kg, 0.0001 to 60 mg/kg, 0.0001 to 50 mg/kg, 0.0001 to 40 mg/kg, 0.0001 to 30 mg/kg, 0.0001 to 20 mg/kg, 0.0001 to 10 mg/kg, 0.0001 to 5 mg/kg, 0.0001 to 4 mg/kg, 0.0001 to 3 mg/kg, 0.0001 to 2 mg/kg, 0.0001 to 1 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the conditions to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

The dosing frequency of the administration of the pharmaceutical composition of the disclosure depends on the nature of the therapy and the particular disease being treated. The subject can be treated at regular intervals, such as weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

As used herein, the terms “co-administration”, “co-administered” and “in combination with”, referring to the a pharmaceutical composition of the disclosure and one or more other therapeutic agents, is intended to mean, and does refer to and include the following: simultaneous administration of such combination of a polypeptide construct of the disclosure and therapeutic agent(s) to a subject in need of treatment, when such components are formulated together into a single dosage form which releases said components at substantially the same time to said subject; substantially simultaneous administration of such combination of a composition of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of a polypeptide construct of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of a polypeptide construct of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present disclosure provides a method for treating cancer or cancer metastasis in a subject, comprising administering a therapeutically effective amount of the pharmaceutical compositions of the invention in combination with a second therapy, including, but not limited to immunotherapy, cytotoxic chemotherapy, small molecule kinase inhibitor targeted therapy, surgery, radiation therapy, and stem cell transplantation. For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of the combination methods described herein.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

In various embodiments, a second anti-cancer agent, such as a chemotherapeutic agent, will be administered to the patient. The list of exemplary chemotherapeutic agent includes, but is not limited to, daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

In various embodiments, the combination therapy methods of the present disclosure may further comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints), such as including, but not limited to antibody to, CTLA-4, PDL-1, CD40, OX-40, CD137, GITR, LAG3, TIM-3, SIRPα, CD47, GITR, ICOS, CD27, Siglec 7, Siglec 8, Siglec 9, Siglec 15, VISTA, CD276, CD272, TIM-3, and B7-H4; treatment using bispecific T cell engaging antibodies (BITE®) such as blinatumomab; treatment involving administration of biological response modifiers such as IL-2, IL-7, IL-10, IL-12, GM-CSF, IFN-α, IFN-β, IFN-γ, TGF-β antagonist or TGF-β trap; treatment using therapeutic vaccines, including, but not limited to oncolytic virus, such as T-vec, or therapeutic vaccine, such as sipuleucel-T; treatment using dendritic cell vaccines, or tumor antigen peptide or neoantigen vaccines; treatment using chimeric antigen receptor (CAR)-T cells; treatment using CAR-NK cells; treatment using NK cell; treatment using iPS induced-NK cells; treatment using iPS induced-T cells; treatment using iPS induced CAR-T or iPS induced CAR-NK cells treatment using tumor infiltrating lymphocytes (TILs); treatment using adoptively transferred anti-tumor T cells (ex vivo expanded and/or TCR-T cells); treatment using TALL-104 cells; and treatment using immunostimulatory agents such as Toll-like receptor (TLR) agonists CpG, TLR7, TLR8, TLR9, and vaccine such as Bacille Calmette-Guerine (BCG), and imiquimod; wherein the combination therapy provides increased effector cell killing of tumor cells, i.e., a synergy exists between the VitoKine constructs and the immunotherapy when co-administered.

In various embodiments, the combination therapy comprises administering a polypeptide composition of the disclosure and the second agent composition simultaneously, either in the same pharmaceutical composition or in separate pharmaceutical composition. In various embodiments, a polypeptide composition and the second agent composition are administered sequentially, i.e., an immunocytokine or a VitoKine construct composition is administered either prior to or after the administration of the second agent composition. In various embodiments, the administrations of an immunocytokine or a VitoKine construct composition and the second agent composition are concurrent, i.e., the administration period of an immunocytokine or a VitoKine construct composition and the second agent composition overlap with each other. In various embodiments, the administrations of, an immunocytokine or a VitoKine construct composition and the second agent composition are non-concurrent. For example, in various embodiments, the administration an immunocytokine or a VitoKine construct composition is terminated before the second agent composition is administered. In various embodiments, the administration of a second agent composition is terminated before an immunocytokine or a VitoKine construct composition construct composition is administered.

The following examples are offered to more fully illustrate the disclosure but are not construed as limiting the scope thereof.

### Example 1

### PD1 Blocking Antibody Sequence Optimization

The current invention aims to optimize the variable domain sequences of pembrolizumab to enhance the score of similarity to the human germline sequences, a measure for their “humanness”. This enhancement could potentially lower the immunogenicity risk. Additionally, the inventors used a human VH3 family germline sequence, which, despite being less homologous, is more prevalent and behaves better, as an alternative acceptor framework. This was done with the aim of improving the biophysical properties of the resulting humanized antibody, ensuring it retains full activity, and enhancing its sequence humanness.

Pembrolizumab was humanized by CDR grafting technology, using the most homologous human antibody sequences available in RCSB protein databank as the acceptor human frameworks. The frameworks found in GenBank under accession numbers AB063829 (SEQ ID NO: 40) and M29469 (SEQ ID NO: 41) were used as the acceptor human frameworks for the heavy chain variable domain (VH) and light chain variable domain (VL), respectively (Carven G J et al., U.S. Pat. No. 8,354,509B2). However, pembrolizumab only shares 79.6% sequence identity to IGHV1-2, its closest human germline, according to a comparison of the variable region exons using the International Immunogenetics Information System (IMGT) DomainGapAlign tool (www.imgt.org). A similarity score to the human germline sequence was proposed as a defining criterion for therapeutic antibodies by the international nonproprietary names (INN) group of WHO in 2014, presumably based on the notion that higher similarity could suggest reduced immunogenicity. The low score of similarity, or “degree of humanness” (Abhinandan K R et al., J Mol Biol (2007) 369:852-62) of pembrolizumab's heavy chain could be due to the poor level of conservation between the mouse CDRs and their human sequence equivalents, the necessity to retain a few structurally important mouse framework residues to recapitulate antigen binding, and the preservation of unique somatic mutations in the human framework sequence AB063829.

To enhance the degree of humanness of pembrolizumab, certain CDR residues were targeted for substitution with their equivalent residues from the closest human germline sequences. This method is herein referred to as CDR germlining. While avoidance of CDR perturbation has traditionally been a central principle humanized Abs design, only a limited number of CDR residues engage in direct antigen interaction. Therefore, certain CDR residues may be replaced without compromising the activity of the antibody. According to the Kabat numbering scheme, CDRs are defined as amino acid residues 24-34 (CDR-L1), 50-56 (CDR-L2), 89-97 (CDR-L3), 31-35b (CDR-H1), 50-65 (CDR-H2), and 95-102 (CDR-H3).

As for the CDR3 sequences, a portion of the light chain CDR3 (CDR-L3) and the entire heavy chain CDR3 (CDR-H3) were not part of the germline sequence's variable region exons V region. Consequently, there are no human germline residues available to substitute the mouse CDR counterparts. Additionally, CDR3s, especially CDR-H3, are highly variable and vital for antigen binding and functional activity, making it crucial to preserve their conformations. As such, both CDR-L3 (QHSRDLPLT; SEQ ID NO: 25) and CDR-H3 (RDYRFDMGFDY; SEQ ID NO: 33) of pembrolizumab were excluded from the CDR germlining process.

The sequences of pembrolizumab's CDR-L1 and CDR-L2 were aligned to their counterparts from the closest human germline IGKV3D-11 (GenBank accession #X17264; SEQ ID NO: 39). The alignments are shown in Table 6A. Likewise, the alignments of CDR-H1 and CDR-H2 sequences of pembrolizumab with the closest human germline sequence, IGHV1-2 (GenBank accession #X62106; SEQ ID NO: 37), are displayed in Table 6B.

TABLE 6A
Alignments of the CDR-L1 and CDR-L2 sequences
of pembrolizumab with IGKV3D-11

| Antibody | CDR-L1 | CDR-L2 |
|---|---|---|
| Pembrolizumab | RASKGVSTSGYSYLH | LASYLES |
| IGKV3D-11 | RASQGV----SSYLA | DASNRAT |

TABLE 6B
Alignments of the CDR-H1 and CDR-H2 sequences
of pembrolizumab with IGHV1-2

| Antibody | CDR-H1 | CDR-H2 |
|---|---|---|
| Pembrolizumab | NYYMY | GINPSNGGTNFNEKFKN |
| IGHV1-2 | GYYMH | WINPNSGGTNYAQKFQG |

“-” indicates sequence gap.
Residues in bold and italic represent those interacting with PD1, as per the complex structure (Horita S. et al., Sci Rep (2016) 6:35297).
Underscored residues are subject to the CDR germlining process.

Multiple pembrolizumab CDR residues directly engage polar interactions with PD1, e.g., hydrogen bond and salt bridge (Horita, S. et al. Sci. Rep (2016). 6:35297). The contacting residues in or around both VL and VH CDR1 and CDR2 include ^{L}Ser28, ^{L}Tyr30 in CDR-L1, ^{L}Tyr49 immediately prior to CDR-L2, ^{L}Tyr53 in CDR-L2, ^{H}Tyr33, ^{H}Tyr35 in CDR-H1, ^{H}Asn52, ^{H}Ser53, ^{H}Asn54, ^{H}Thr57, ^{H}Asn58 in CDR-H2 (where the superscripted letter ‘L’ donates light chain, and ‘H’ refers to heavy chain). Except for ^{L}Tyr49, which is not a CDR residue and not shown, all the antigen-interacting CDR residues mentioned above are in bold and italic in Tables 6A and 6B. Among the CDR residues that differ from the germline sequences, CDR-L1 residues ^{L}Lys27, ^{L}His34, CDR-L2 residues ^{L}Leu54, ^{L}Glu55, CDR-H2 residues ^{H}Phe59, ^{H}Asn60, ^{H}Glu61, ^{H}Lys64, and ^{H}Asn65 (underscored in Tables 6A and 6B) were selected for CDR germlining. They were replaced with their respective human germline equivalents with the following amino acid substitutions: ^{L}K27Q, ^{L}H34A, ^{L}L54R, ^{L}E55A, ^{H}F59Y, ^{H}N60A, ^{H}E61Q, ^{H}K64Q, and ^{H}N65G either individually or in combination. Other CDR residues were reserved to avoid any disruption of the antigen-interacting residues.

For CDR-H1 (NYYMY; SEQ ID NO: 26), only a single residue, ^{H}Asn31, is eligible for CDR germlining, to be replaced with its equivalent residue, glycine, in the human germline sequence. Other residues were kept either because they engage in direct antigen interaction with PD1 (^{H}Tyr33 and ^{H}Tyr35) or because they are conserved between the mouse and human germline sequences (^{H}Tyr32 and ^{H}Met34). However, considering CDR-H1's short length of only five amino acids and the fact that two residues have a direct interaction with the antigen, any amino acid alteration could potentially disturb the CDR confirmation and impact the antibody's activity. Consequently, ^{H}Asn31 was not modified through CDR germlining, and CDR-H1 is fully reserved.

In addition to the low level of conservation between the mouse CDRs and their corresponding human germline sequences, the pembrolizumab heavy chain frameworks (FRs) also contain multiple non-germline residues. They arose due to the retention of the unique somatic mutations in the acceptor framework sequence AB063829. These somatic mutations, including ^{H}Val9 in FR-H1, ^{H}Thr76, ^{H}Lys82a, ^{H}Gln83, ^{H}Phe84 in FR-H3 and ^{H}Thr108 in FR-H4, are not considered structurally important. Replacement with their respective germline counterparts, ^{H}V9A, ^{H}T76S, ^{H}K82aS, ^{H}Q83R, ^{H}F84S, ^{H}T108L, leads to a considerable improvement in the similarity score to the human germline sequence, without disturbing the CDR conformation or altering the antibody's activity.

Moreover, IGHV3-23 (SEQ ID NO: 38) was used as an alternative acceptor framework to investigate whether utilizing a human acceptor framework with substantially lower sequence homology, but superior biophysical attributes could enhance the biophysical properties of the resultant humanized antibody without compromising its functional activity. IGHV3-23 belongs to the human antibody heavy chain germline VH3 family, which is the most common VH family in the human repertoire. It is also the most prevalent among the marketed human monoclonal antibodies and is widely acknowledged for its superior drug-like properties. Given that the CDR conformation was exquisitely sensitive to the chemical environment of the surrounding framework, a few structurally significant framework residues in pembrolizumab, which differ from their VH3 germline family equivalents, were selected for reverse mutation to their corresponding pembrolizumab equivalents. Furthermore, several CDR-H2 residues were targeted for CDR germlining using IGHV3-23 CDR-H2 as a template to improve the similarity score to the human germline sequence.

Alignments of the CDR-H1 and CDR-H2 sequences of pembrolizumab with IGHV3-23 are displayed in Table 6C. Six CDR-H2 residues, ^{H}Phe59, ^{H}Asn60, ^{H}Glu61, ^{H}Lys62, ^{H}Phe63, and ^{H}Asn65 (underscored in Table 6C; the superscripted letter ‘L’ donates light chain, and ‘H’ refers to heavy chain) were selected for CDR germlining with the following amino acid substitutions: ^{H}F59Y, ^{H}N60A, ^{H}E61D, ^{H}K62S, ^{H}F63V, and ^{H}N65G. CDR-H1 was excluded from the CDR germlining process for the reason described earlier.

TABLE 6C
Alignments of the CDR-H1 and CDR-H2 sequences
of pembrolizumab with IGHV3-23

| Antibody | CDR-H1 | CDR-H2 |
|---|---|---|
| Pembrolizumab | NYYMY | GINPSNGGTNFNEKFKN |
| IGHV3-23 | SYAMS | AISGSGGSTYYADSVKG |

Residues in bold and italic represent those interacting with PD1, as per the complex structure (Horita S. et al., Sci Rep (2016) 6: 35297).
Underscored residues are subject to the CDR germlining process.

Two framework residues, ^{H}Thr30 and ^{H}Arg94, were considered structurally significant and were preserved without alteration to their corresponding germline equivalents, ^{H}Ser30 and ^{H}Lys94. Five additional IGHV3 framework residues, ^{H}Val48, ^{H}Ser49, ^{H}Ile69, ^{H}Arg71, and ^{H}Asn73, which belong to the vernier zone (U.S. Pat. Nos. 5,821,337 and 5,859,205) and may be of structural significance, were reverted to their corresponding pembrolizumab residues, ^{H}Met48, ^{H}Gly49, ^{H}Leu69, ^{H}Thr71, and ^{H}Ser73, either individually or in combination. The importance of specific framework amino acid residues was assessed experimentally. The number of reverse mutations was minimized to ensure the highest similarity score to the germline sequence without negatively affecting antibody activity.

All the optimized antibody sequences were expressed as full length antibody with a kappa light chain constant region containing the sequence set forth in SEQ ID NO: 34 and a modified IgG1 heavy chain constant region containing the sequence set forth in SEQ ID NO: 35. Table 7 lists the SEQ ID NOS of the VL, VH, CDR-L1, CDR-L2, and CDR-H2 of the exemplary optimized PD1 blocking antibodies along with the Reference Antibody (P-0734), comprising VL and VH sequences set forth in SEQ ID NO: 2 and SEQ ID NO: 6, respectively. All antibodies of the present invention comprise identical CDR-L3 (SEQ ID NO: 25), CDR-H1 (SEQ ID NO: 26), and CDR-H3 (SEQ ID NO: 33).

TABLE 7
Exemplary PD1 blocking antibodies
resulted from CDR and FR germlining

| | SEQ ID NO | | | | |
|---|---|---|---|---|---|
| PD1 Antibody | VL | VH | CDR-L1 | CDR-L2 | CDR-H2 |
| P-0734 | 2 | 6 | 19 | 22 | 27 |
| P-1127 | 3 | 6 | 20 | 23 | 27 |
| P-1129 | 4 | 6 | 20 | 24 | 27 |
| P-1148 | 4 | 7 | 20 | 24 | 28 |
| P-1150 | 5 | 7 | 21 | 24 | 28 |
| P-1151 | 4 | 8 | 20 | 24 | 29 |
| P-1153 | 5 | 8 | 21 | 24 | 29 |
| P-1174 | 3 | 7 | 20 | 23 | 28 |
| P-1175 | 2 | 10 | 19 | 22 | 31 |
| P-1176 | 2 | 11 | 19 | 22 | 31 |
| P-1177 | 2 | 12 | 19 | 22 | 31 |
| P-1178 | 2 | 13 | 19 | 22 | 31 |
| P-1181 | 2 | 14 | 19 | 22 | 30 |
| P-1193 | 3 | 15 | 20 | 23 | 32 |
| P-1194 | 3 | 11 | 20 | 23 | 31 |
| P-1198 | 3 | 16 | 20 | 23 | 32 |
| P-1199 | 3 | 17 | 20 | 23 | 32 |
| P-1201 | 3 | 18 | 20 | 23 | 32 |
| P-1238 | 3 | 13 | 20 | 23 | 32 |
| P-1271 | 3 | 9 | 20 | 23 | 28 |

### Example 2

### Construction, Production, and Purification of the Optimized PD1 Blocking Antibodies

All genes were codon optimized for expression in mammalian cells, and they were synthesized and subsequently subcloned into the recipient mammalian expression vectors through the service of GenScript. Protein expression is driven by a CMV promoter, and a synthetic SV40 polyA signal sequence is positioned at the 3′ end of the coding sequence. A leader sequence was engineered at the N-terminus of the constructs to ensure appropriate signaling and processing for secretion.

The antibodies were produced by co-transfecting vectors harboring light chain and heavy chain with a 1:1 ratio in ExpiCHO cells (ThermoFisher) following the manufacturer's instructions. On the day of transfection, ExpiCHO cells were diluted to 6×10⁶ cells/mL in ExpiCHO™ expression medium (ThermoFisher). Expression vectors, totaling 0.8 μg DNA/mL culture volume, were mixed with cold OptiPRO™ medium (40 μL/mL cell culture). Following the addition of ExpiFectamine™ CHO reagent at 3.2 μL/mL cell culture, the solution was gently mixed and subsequently incubated for 5 min at room temperature. The ExpiFectamine™ CHO/plasmid DNA complexes were then slowly transferred to the cells and incubated at 37° C. in a shaker incubator at 130 rpm with 8% CO₂ atmosphere. ExpiFectamine™ CHO enhancer (6 L/mL cell culture) and ExpiCHO™ feed (240 L/mL cell culture) were added to the flask with gentle swirling 18-22 hours post transfection. After 8 days of cultivation, the supernatant was harvested for purification by centrifugation for 20 min at 2200 rpm, followed by sterile filtered using a 0.22 μm filter (Corning).

The secreted antibody was purified from cell culture supernatants using Protein A affinity chromatography. Cell culture supernatant was loaded onto a MabSelect SuRe 5-mL column (Cytiva) equilibrated with 5 column volumes (CV) of phosphate buffered saline, pH 7.2 (ThermoFisher). Unbound protein was removed by washing with 5 CVs of PBS, pH 7.2, and target protein was eluted with 25 mM sodium citrate, 25 mM sodium chloride buffer, pH 3.2. Antibody solution was neutralized by adding 3% of 1 M Tris buffer, pH 10.2 followed by concentration and buffer exchange to PBS, pH 7.2 using Amicon® Ultra-15 Ultracel with 10 KDa MWCO (Merck Millipore).

The purity and molecular weight of the purified antibodies were analyzed by SDS-PAGE, both with and without a reducing agent, and then stained with Coomassie (Imperial™ protein stain, ThermoFisher). The SurePAGE™ Pre-Cast gel system (8-16% Bis-Tris, GenScript) was used according to the manufacturer's instruction. The aggregate content of the antibodies was analyzed on an Agilent 1200 high-performance liquid chromatography (HPLC) system. Samples were injected into an AdvanceBio size-exclusion column (300 Å, 4.6×150 mm, 2.7 μm, LC column, Agilent) using 150 mM sodium phosphate buffer, pH 7.0 as the mobile phase at 25° C.

The antibody concentration of purified protein samples was determined by measuring the absorbance at 280 nm using a Nanodrop spectrophotometer (ThermoFisher) divided by the molar extinction coefficient calculated based on its amino acid sequence. Endotoxin level of purified protein samples were measured using Endosafe nexgen-PTS (Charles River) as per the manufacturer's instruction.

### Example 3

### Assays to Evaluate the Biological Activities of the Optimized PD1 Blocking Antibodies

Antibodies of the invention were tested for their antigen binding activity by well-known methods such as enzyme-linked immunosorbent assay (ELISA). Briefly, Nunc Maxisorp plates (ThermoFisher) were coated with recombinant human PD1 protein in bicarbonate buffer, pH 9.4 (ThermoFisher), overnight at 4° C., using 1 μg of antigen per well (100 μL/well). After triple washing with PBS/0.05% Tween 20, plates were incubated with SuperBlock (ThermoFisher) for two hours at room temperature to block nonspecific binding. The PD1 antibodies, serially diluted three-fold with blocking buffer (PBS with 1% bovine serum albumin) were added to the plates (100 μL/well) post washing and incubated at room temperature for one hour. After another washing, antibodies were detected by incubating with a horseradish peroxidase (HRP)-conjugated goat anti-human IgG Fc antibody (ThermoFisher) diluted 1:5000 in blocking buffer (100 μL/well) for one hour at room temperature. After a final wash, TMB substrate (ThermoFisher) at 100 μL/well was added. Plates were sealed and left to incubate in dark for 5-20 minutes. The reaction was stopped by adding 2N sulfuric acid (Ricca Chemical) (50 μL/well), and the absorbance was measured at 450 nm with a plate reader. The curves were plotted, and the half-maximal effective concentration (EC₅₀) values were calculated using GraphPad Prism software.

Additionally, HEK 293T cells stably expressing human PD1 gene (Crown Bioscience) was used to determine the cell-based binding strength of the optimized PD1 antibodies by flow cytometry. After harvesting, HEK293-hPD1 cells were seeded into a 96-well U-bottom plate at 1×10⁵ cells/well (100 μL), incubated with Fc block (1:50) for 20 minutes at 4° C., and subsequently washed with FACS buffer (PBS, 1% FBS). Cells were then treated with three-fold serial dilutions of each antibody at concentrations ranging from 0.01-100 nM in FACS buffer for 30 minutes at 4° C. Subsequently, cells were washed twice with FACS buffer to remove unbound molecules, and 40 UL of the 1:100 diluted PE-labeled goat anti-human Fc secondary antibody (eBiosciences) was added to the cells. Following a 30-minute incubation at 4° C. and another double wash with FACS buffer, antibodies bound to the cells were detected with PE-labeled secondary antibody by flow cytometry (BD ACCURI-C6), and EC₅₀ values were calculated using GraphPad Prism software.

Furthermore, a thaw-and-use format of Promega luciferase reporter assay, a biologically relevant mechanistic-based assay, was used to measure the potency in blocking PD1 interaction by PD1 antibodies. Cell thawing and plating procedures were followed exactly as described in the manufacturer's protocol.

Briefly, one vial (0.5 mL) of PD-L1 aAPC/CHO-K1 cells were thawed and mixed with 14.5 mL cell recovery medium (90% Ham's F12/10% FBS). Next, 100 μL of this cell suspension was added to the inner 60 wells of two 96-well flat-bottom assay plates, while perimeter wells received 100 μL of cell recovery medium. After overnight incubation at 37° C. and 5% CO2, the medium was discarded. The inner wells received 40 μL of 3-fold serially diluted compounds, while the perimeter wells got 80 μL of assay buffer (99% RPMI 1640/1% FBS). Subsequently, one vial (0.5 mL) of PD1 effector cells were thawed and mixed with 5.9 mL of assay buffer, and 40 μL of this mixture was added to the inner wells. After a 6-hour incubation at 37° C., 5% CO2, and a 7-minute equilibration at room temperature, 80 μL of Bio-Glo™ reagent was added to all wells. The plates were then incubated at room temperature for 10 minutes with shaking. The resulting luminescence was measured using a luminescence plate reader (BioTek synergy h1).

Background was calculated by averaging relative light units (RLU) of perimeter wells. Fold Induction was determined as the RLU of the antibody sample minus background, divided by the RLU of the control samples (without antibody) minus background, or Fold Induction=RLU (antibody-background))/(RLU (no antibody ctrl-background). Finally, EC50 values were determined using the curves fitted using GraphPad Prism software.

### Example 4

### Evaluation of the PD1 Antibodies Comprising Germlining Modifications Based on the Closest Human Germline Sequences

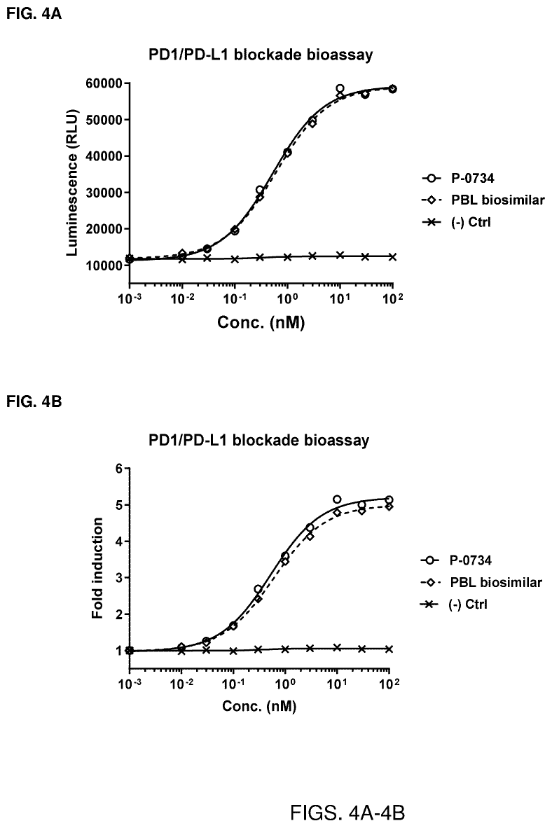

Firstly, the effectiveness of P-0734 in inhibiting the PD1/PD-L1 interaction was compared with that of the pembrolizumab (PBL) biosimilar. While P-0734 and PBL biosimilar share the identical variable domains, they differ in their heavy chain constant region. PBL contains an IgG4 constant chain containing S228P mutation (SEQ ID NO: 36), whereas P-0734 has an IgG1 constant chain with L234A/L235A/G237A mutations (SEQ ID NO: 35) to abrogate Fc effector functions. As shown in FIG. 4, P-0734 and PBL biosimilar were equally potent in blocking the interaction between PD1 and PD-L1. This result was expected and confirms that the ability to block PD1 is determined by the variable domain sequences, and not by the immunoglobulin classes. P-0734 faithfully recapitulates the potency of PBL biosimilar in blocking the PD1/PD-L1 interaction and is herein referred to as the Reference Antibody.

The impacts of CDR germlining substitutions ^{L}H34A in CDR-L1 and ^{H}F60Y in CDR-H2 were evaluated using antibodies with slightly different mutational contexts. These antibodies, P-1148, P-1150, P-1151, and P-1153, all contain germlining substitutions ^{L}K27Q in CDR-L1, ^{L}L54R, ^{L}E55A in CDR-L2, and ^{H}N60A, ^{H}E61Q, ^{H}K64Q, ^{H}N65G in CDR-H2. P-1150 includes an additional ^{L}H34A substitution in CDR-L1, P-1151 has an extra ^{H}F59Y substitutions in CDR-H2, and P-1153 harbors both ^{L}H34A and ^{H}F59Y changes in addition. Table 8 provides a list of the CDR germlining substitutions for these exemplary PD1 blocking antibodies.

TABLE 8
CDR germlining substitutions of exemplary PD1 blocking antibodies

| PD1 Ab | CDR-L1 | CDR-L2 | CHR-H2 |
|---|---|---|---|
| P-1148 | ^{L}K27Q | ^{L}L54R, ^{L}E55A | ^{H}N60A, ^{H}E61Q, ^{H}K64Q, ^{H}N65G |
| P-1150 | ^{L}K27Q, ^{L}H34A | ^{L}L54R, ^{L}E55A | ^{H}N60A, ^{H}E61Q, ^{H}K64Q, ^{H}N65G |
| P-1151 | ^{L}K27Q | ^{L}L54R, ^{L}E55A | ^{H}F59Y, ^{H}N60A, ^{H}E61Q, ^{H}K64Q, ^{H}N65G |
| P-1153 | ^{L}K27Q, ^{L}H34A | ^{L}L54R, ^{L}E55A | ^{H}F59Y, ^{H}N60A, ^{H}E61Q, ^{H}K64Q, ^{H}N65G |

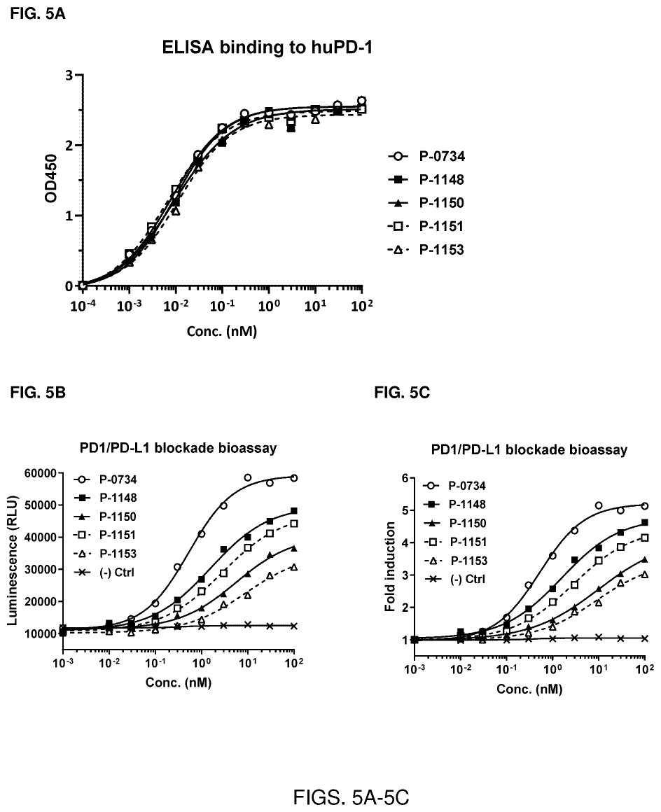

As illustrated in FIGS. 5B & 5C and summarized in Table 9, the CDR-L1 germlining substitution ^{L}H34A consistently led to a reduction in PD1 blockade potency (EC₅₀) of approximately 3.5-fold, along with a 25% reduction in both Eₘₐₓ (maximum effect/luminescence signal) and fold induction, regardless of the presence (P-1151 vs P-1153) or absence (P-1148 vs P-1150) of the ^{H}F59Y substitution. The impact of ^{H}F59Y germlining substitution was similarly assessed, with the data shown in FIGS. 5B & 5C and summarized in Table 9. Irrespective of whether the ^{L}H34A substitution is present (P-1150 vs P-1153) or absent (P-1148 vs P-1151, the ^{H}F59Y CDR germlining substitution resulted in a consistent albeit modest reduction in both potency (EC₅₀; reduced by about 1.8-fold) and signal (10-15% decrease in both Eₘₐₓ and fold induction). Compared to P-0734, the cumulative CDR germlining substitutions in P-1153 ended up with a nearly 20-fold decrease in PD1 blockade potency (EC₅₀) and 50% reduction in Eₘₐₓ. As a result, both ^{L}H34A and ^{H}F59Y substitutions were deemed detrimental in this particular framework and the original CDR residues, ^{L}His34 and ^{H}Phe59, will be preserved.

However, despite the notable differences in potency in blocking the PD1/PD-L1 interaction, all four optimized PD1 antibodies and the Reference Antibody, P-0734, displayed nearly identical binding strength with an EC₅₀ value close to 100 pM (FIG. 5A and Table 9).

TABLE 9
ELISA binding and PD1 blocking activity
of exemplary PD1 blocking antibodies

| | ELISA binding | PD1 blocking activity (reporter assay) | | |
|---|---|---|---|---|
| | EC₅₀ | EC₅₀ | Vmax | Fold |
| PD1 Ab | (pM) | (nM) | (RLU) | induction |
| P-0734 | 82 | 0.53 | 58381 | 5.13 |
| P-1148 | 93 | 1.56 | 48227 | 4.63 |
| P-1150 | 88 | 5.55 | 36537 | 3.49 |
| P-1151 | 65 | 2.83 | 44252 | 4.15 |
| P-1153 | 110 | 9.88 | 30760 | 3.04 |

This piece of data suggested that the mechanistic-based functional assay was capable of discerning minute activity changes that aren't detectable by ELISA binding assay. As such, the luciferase PD1/PD-L1 reporter assay is herein used as the primary tool to characterize and rank PD1 blocking antibodies derived from pembrolizumab via germlining substitutions. It is expected that the derivative antibodies that maintain full functional activity will exhibit identical in vivo efficacy as pembrolizumab.

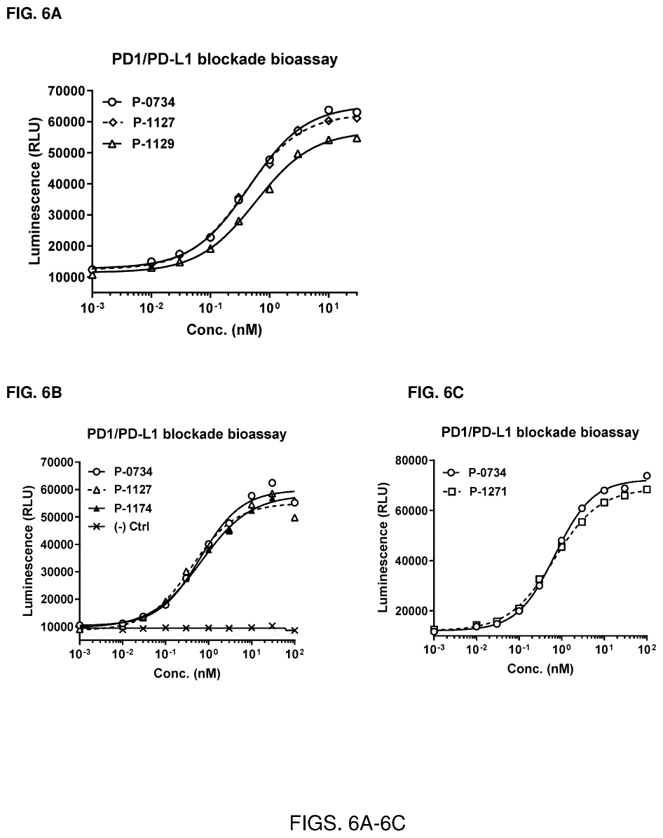

The potential negative effects of CDR-L2 germlining substitution ^{L}E55A was further assessed by comparing P-1127 and P-1129. Both these molecules contain ^{L}K27Q and K54E germline substitutions in the light chain CDRs, with the only sequence difference being the extra CDR-L2 substitution, ^{L}E55A, in P-1129. As demonstrated in FIG. 6A, P-1129 displayed a minor yet appreciable reduction in potency (EC_{50=0.39} nM and 0.58 nM for P-1127 and P-1129, respectively) and a marginal 10% decrease in Eₘₐₓ. Hence, the ^{L}E55A amino acid substitution was deemed adverse and the original residue, ^{L}Glu55, will be preserved.

P-1174, harboring a total of 6 CDR germlining substitutions, ^{L}K27Q, ^{L}L54R, ^{H}N60A, ^{H}E61Q, ^{H}K64Q, and ^{H}N65G, exhibited identical PD1 blockade activity as P-1127 and P-0734 with EC₅₀ of 0.64 nM, 0.54 nM, and 0.67 nM for P-0734, P-1127, and P-1174, respectively (FIG. 6B). Additionally, P-1174 was derived from P-114 by eliminating one CDR germlining substitution, ^{L}E55A. When compared to P-0734, P-1174 exhibited higher potency than P-1148 (refer to FIG. 5B & FIG. 6B). This piece of data further collaborated the conclusion that the original CDR residues, ^{L}Glu55, should not be altered.

Besides the low conservation between the mouse CDRs and their human germline counterparts, multiple non-germline residues in the pembrolizumab VH framework also contributed to the low sequence similarity score with the germline. These non-germline residues, originating from the unique somatic mutations preserved in the acceptor framework sequence, including ^{H}Val9 in FR-1, ^{H}Thr76, ^{H}Lys82a, ^{H}Gln83, ^{H}Phe84 in FR-3 and ^{H}Thr108 in FR-4, are considered not to be structurally significant. To further enhance the sequence similarity score with the germline or degree of humanness, these non-germline residues in the P-1174 framework were replaced with their respective germline equivalents, ^{H}V9A, ^{H}T76S, ^{H}K82aS, ^{H}Q83R, ^{H}F84S, ^{H}T108L, resulting in P-1271. As expected, P-1271 displayed the same PD1 blocking activity as the Reference Antibody, P-0734 (FIG. 6C) with EC₅₀ values of 0.66 nM for P-1271 and 0.70 for P-0734, respectively.

In conclusion, CDR germlining substitutions, ^{L}K27Q, ^{L}L54R, ^{H}N60A, ^{H}E61Q, ^{H}K64Q, and ^{H}N65G, in P-1174 and P-1271 enhanced antibody sequence degree of humanness without compromising the potency in blocking the PD1/PD-L1 interaction. Additional six framework germlining substitutions in P-1271 further augmented the score of similarity to the closest human germline sequences. Table 10 lists the germlining substitutions and similarity scores to the closest human germline sequences of P-1174 and P-1271 in comparison to the Reference Antibody, P-0734.

TABLE 10
Germlining substitutions and similarity scores of the exemplary optimized PD1
antibodies, P-1174 and P-1271, with the closest human germline sequences

| | | | VL similarity | VH similarity |
|---|---|---|---|---|
| | CDR germling | FR germlining | score to | score to |
| PD1 Ab | substitutions | substitutions | IGKV3D-11 | IGHV1-2 |
| P-0734 | N/A | N/A | 85.1% | 79.6% |
| P-1174 | ^{L}K27Q, ^{L}L54R, ^{H}N60A, | N/A | 87.2% | 83.7% |
| | ^{H}E61Q, ^{H}K64Q, and ^{H}N65G | | | |
| P-1271 | ^{L}K27Q, ^{L}L54R, ^{H}N60A, | ^{H}V9A, ^{H}T76S, | 87.2% | 88.8% |
| | ^{H}E61Q, ^{H}K64Q, and ^{H}N65G | ^{H}K82aS, ^{H}Q83R, | | |
| | | ^{H}F84S, ^{H}T108L | | |

### Example 5

### Evaluation of the PD1 Antibodies Comprising Germlining Modifications Based on a More Prevalent Human Germline Family (VH3)

The adoption of framework germlining substitutions based on the human antibody heavy chain germline IGHV3-23 (SEQ ID NO: 38) was investigated to exam whether an antibody framework with substantially lower sequence homology, but superior biophysical properties, could enhance the drug-like properties of the resultant antibody while fully retaining its functional activity. Of the 33 framework germlining substitutions (Table 11A), the importance of the 5 Vernier zone residues, ^{H}V48, ^{H}S49, ^{H}I69, ^{H}R71, and ^{H}N73, were assessed experimentally by reversion mutation to their respective pembrolizumab equivalents, ^{H}V48M, ^{H}S49G, ^{H}I69L, ^{H}R71T, and ^{H}N73S, either individually or in combination. Additionally, six CDR-H2 residues, ^{H}F59Y, ^{H}N60A, ^{H}E61D, ^{H}K62S, ^{H}F63V, and ^{H}N65G, were selected for CDR germlining substitutions with their corresponding residues in IGHV3-23. Table 11B provides a summary of the VH3 germlining substitutions in the exemplary antibodies.

TABLE 11A
VH Framework germlining substitutions
based on IGHV3-23

| VH FRs | Designation | Sequences |
|---|---|---|
| FR1 (1-30) | P-0734 | QVQLVQSGVEVKKPG |
| | | ASVKVSCKASGYTFT |
| | After Germlining | EVQLLESGGGLVQPG |
| | | GSLRLSCAASGFTFT |
| FR2 (36-49) | P-0734 | WVRQAPGQGLEWMG |
| | After Germlining | WVRQAPGKGLEWVS |
| FR3 (66-94) | P-0734 | RVTLTTDSSTTTAY |
| | | MELKSLQFDDTAVYY |
| | | CAR |
| | After Germlining | RFTISRDNSKNTLY |
| | | LQMNSLRAEDTAVYY |
| | | CAR |
| FR4 (103-113) | P-0734 | WGQGTTVTVSS |
| | After Germlining | WGQGTLVTVSS |

The bolded and underlined residues represent a total of 33 framework germlining substitutions.

TABLE 11B
VH CDR germlining and FR reversion mutations based on IGHV3-23

| | | No. of FR | |
|---|---|---|---|
| | CHR-H2 germlining | germlining | Reversion mutation |
| PD1 Ab | substitutions | substitutions | of FR residues |
| P-1175 | ^{H}F59Y, ^{H}N60A, ^{H}E61D, ^{H}N65G. | 33 | N/A |
| P-1181 | ^{H}E61D, ^{H}K62S, ^{H}F63V, ^{H}N65G. | 33 | N/A |
| P-1176/P-1194 | ^{H}F59Y, ^{H}N60A, ^{H}E61D, ^{H}N65G | 30 | ^{H}I69L, ^{H}R71T, ^{H}N73S |
| P-1177 | ^{H}F59Y, ^{H}N60A, ^{H}E61D, ^{H}N65G | 31 | ^{H}V48M, ^{H}S49G |
| P-1178/P-1238 | ^{H}F59Y, ^{H}N60A, ^{H}E61D, ^{H}N65G | 28 | ^{H}V48M, ^{H}S49G, |
| | | | ^{H}I69L, ^{H}R71T, ^{H}N73S |
| P-1193 | ^{H}N60A, ^{H}E61D, ^{H}N65G | 33 | N/A |
| P-1198 | ^{H}N60A, ^{H}E61D, ^{H}N65G | 32 | ^{H}N73S |
| P-1199 | ^{H}N60A, ^{H}E61D, ^{H}N65G | 31 | ^{H}R71T, ^{H}N73S |
| P-1201 | ^{H}N60A, ^{H}E61D, ^{H}N65G | 30 | ^{H}I69L, ^{H}R71T, ^{H}N73S |

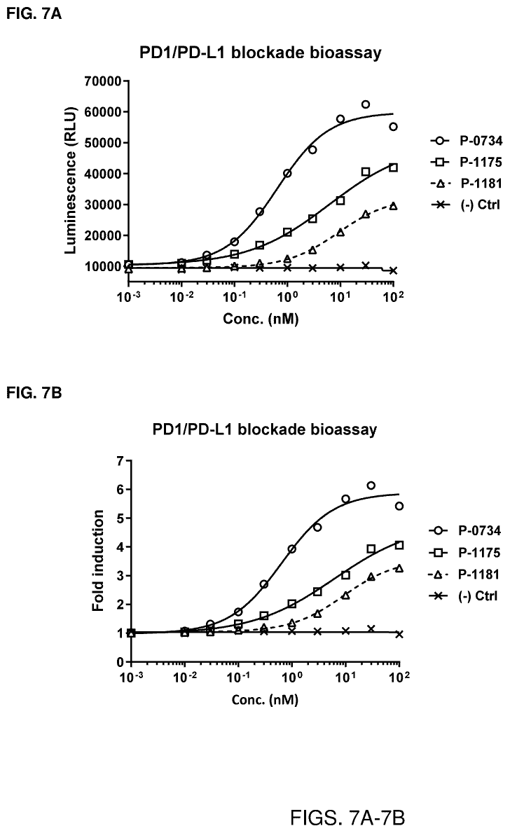

FIG. 7 depicts the PD1 blockade activity of P-1175 and P-1181, differing only in their CDR-H2 germlining substitutions (as shown in Table 11B). Compared to P-0734, both P-1175 and P-1181 exhibited substantially diminished potency in blocking PD1 interaction. Specifically, P-1174 showed a 10-fold reduction in potency (EC₅₀) and 25% decrease in both Eₘₐₓ and fold induction. This was in comparison to P-1181's 15-fold drop in potency and 40-50% decrease in Eₘₐₓ and fold induction (as illustrated in FIGS. 7A & 7B and summarized in Table 12). Since P-1181 displayed a more drastic decline in activity, its two distinct CDR germlining substitutions, ^{H}K62S, ^{H}F63V, were deemed detrimental, hence the original CDR residues, ^{H}Lys62 and ^{H}Phe63, will be preserved. These findings suggested that the significance of individual CDR residues need to be assessed experimentally; even CDR residues that are close to the boundary or are not immediately adjacent to antigen-contacting residues could negatively impact the activity.

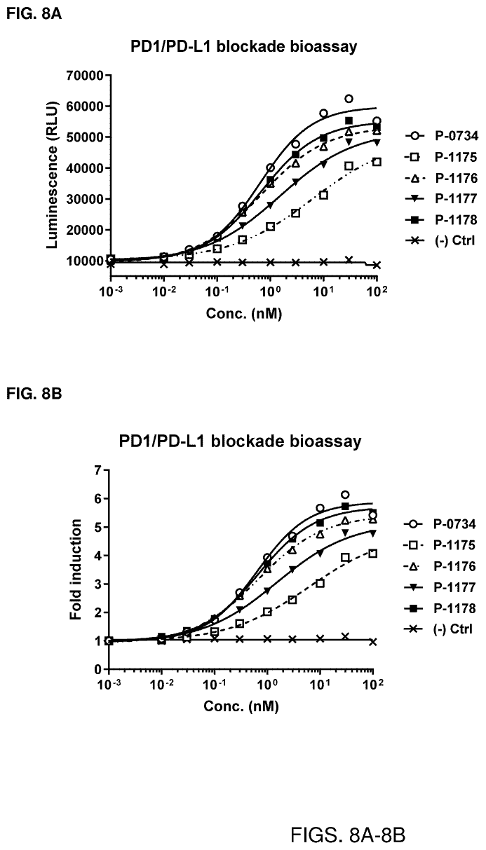

Two to five framework residue reversion mutations were introduced to P-1175, resulting in P-1176, P-1177, and P-1178, as detailed in Table 11. As indicated by the data in FIG. 8, the combined reversion mutations, ^{H}I69L, ^{H}R71T, and ^{H}N73S, in P-1176 effectively restored PD1 blocking activity, almost matching the level of P-0734. Similarly, the activity was significantly reinstated in P-1177 due to the combined reversion mutations, ^{H}V48M, and ^{H}S49G, although not as effectively as in P-1176. Nevertheless, the incorporation of these two reversion mutations (^{H}V48M and ^{H}S49G) into P-1176 did not lead to further enhancement in activity for the resulting antibody, P-1178 (P-1178 vs P-1176 in FIG. 8 and Table 12).

TABLE 12
PD1 blockade activity of exemplary PD1 blocking antibodies

PD1 blocking activity (reporter assay)

| PD1 Ab | EC₅₀ (nM) | Vmax (RLU) | Fold induction |
|---|---|---|---|
| P-0734 | 0.64 | 55200 | 5.42 |
| P-1175 | 6.21 | 41970 | 4.07 |
| P-1181 | 9.18 | 29595 | 3.26 |
| P-1176 | 0.67 | 52093 | 5.27 |
| P-1177 | 1.51 | 48120 | 4.77 |
| P-1178 | 0.68 | 53089 | 5.50 |

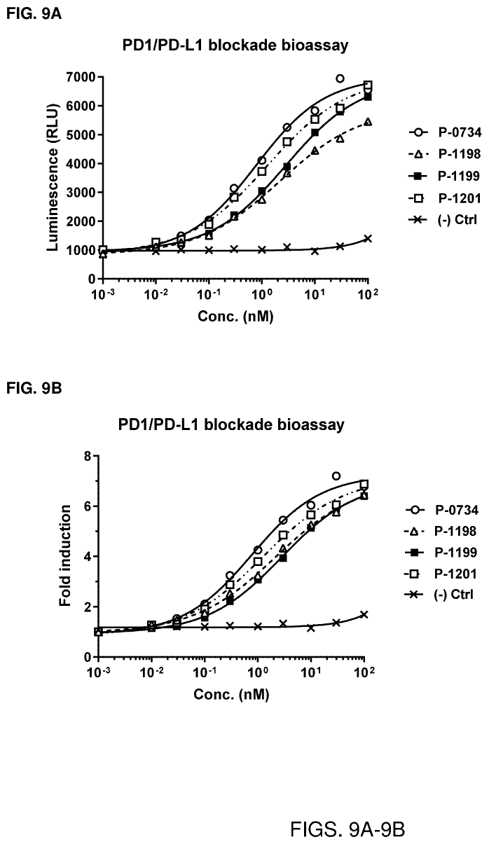

The significance of each of the three FR reversion mutations, ^{H}I69L, ^{H}R71T, and ^{H}N73S were further assessed by comparing PD1 blocking activity of P-1198 (^{H}N73S), P-1199 (^{H}R71T, ^{H}N73S), and P-1201 (^{H}I69L, ^{H}R71T, ^{H}N73S). As demonstrated in FIG. 9, each added reversion mutation led to slight yet evident cumulative increases in PD1 blockade activity. Only the combination of all the three reversion mutations in P-1201 led to nearly fully restored functional activity (with EC₅₀ values of 1.28 nM and 0.78 nM for P-1201 and P-0734, respectively). Thus, all the three reversion mutations, ^{H}I69L, ^{H}R71T, ^{H}N73S, were deemed essential and will be incorporated.

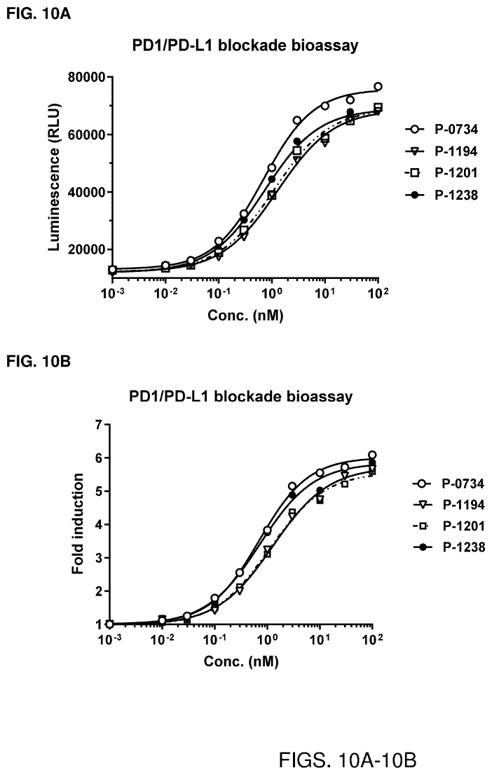

Further, the PD1 inhibitory activity of P-1194, P-1201, and P-1238 were compared and illustrated in FIGS. 10A and 10B. P-1194 and P-1201, differing by only one additional CDR germlining substitution, ^{H}F59Y, displayed identical PD1 blocking potency. This suggests that this particular substitution did not negatively affect the activity, contradicting earlier observation that ^{H}F59Y germlining substitution was detrimental when IGHV1-2 germline sequence was adopted. It is thus postulated that the impact of individual CDR germlining substitution is dependent on the context of the surrounding framework sequences. P-1238 were equally potent as the Reference Antibody, P-0734, with EC₅₀ values of 0.73 nM and 0.70 nM, respectively. Compared to P-1194, the two additional framework reversion mutations, ^{H}V48M, and ^{H}S49G, in P-1238 contributed to slight but discernable improvement in activity.

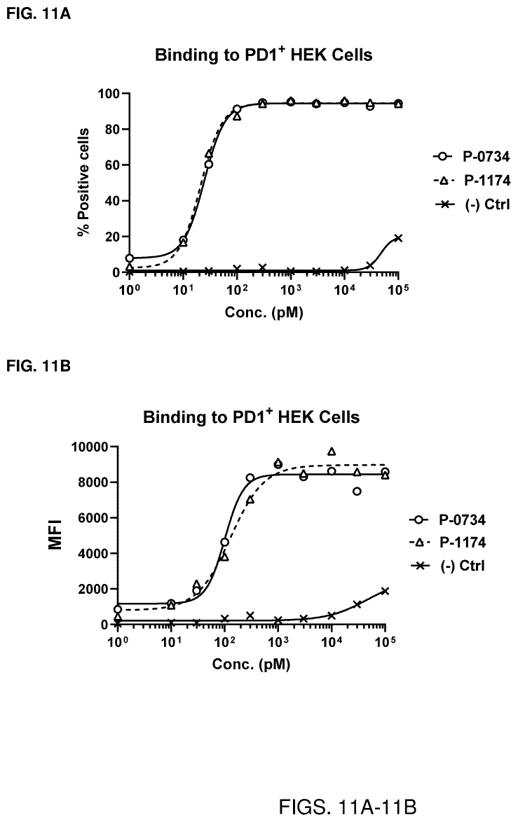

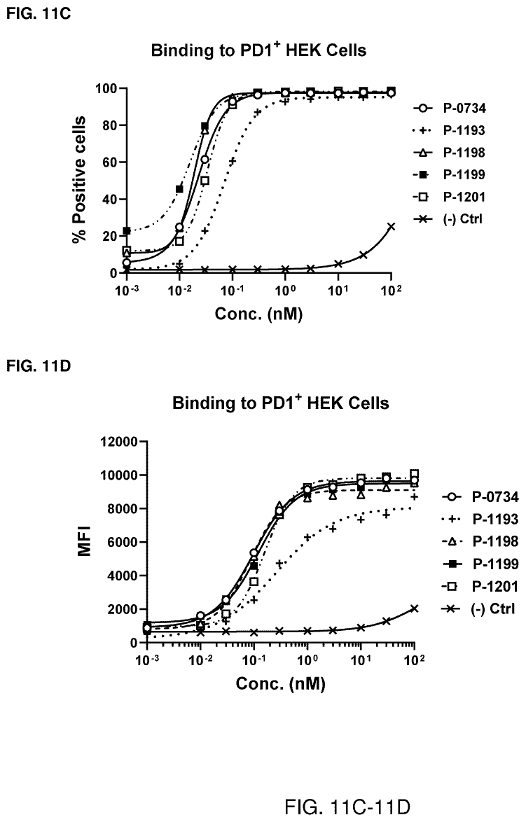

In the final assessment, P-1174, P-1193, P-1198, P-1199, and P-1201 were assessed for their binding strength to PD1⁺ cells (FIG. 11). As anticipated, P-1174, which fully preserved PD1 blocking potency (FIGS. 6C and 6D), displayed a binding affinity to PD1-expressing cells equivalent to the Reference Antibody, P-0734 (FIGS. 11A and 11B). P-1198, P-1199, and P-1201, which contain 1-3 framework reversion mutations, displayed subtle but evident potency difference in blocking PD1 interaction (FIG. 9), but such variations in activity were not detected in the cell-based binding assay. All three compounds demonstrated an equal ability in binding to PD1⁺ cells as P-0734 (FIGS. 11C & 11D and Table 13). Yet, the cell-based binding assay was able to distinguish P-1193, which contains no framework reversion mutation, from other compounds, as shown in FIGS. 11C & 11D and Table 13. The extent of the decrease, though, was less pronounced than what was observed in the blocking assay. The data further corroborate our earlier observation that the mechanistic-based PD1/PD-L1 blocking assay is more sensitive than binding assays in identifying subtle activity differences.

TABLE 13
Binding strength of exemplary optimized
PD1 blocking antibodies to PD1⁺ cells

PD1+ cell binding EC₅₀ (pM)

| PD1 Ab | % Positive cells | MFI |
|---|---|---|
| P-0734 | 23 | 97 |
| P-1193 | 70 | 259 |
| P-1198 | 19 | 87 |
| P-1199 | 16 | 120 |
| P-1201 | 33 | 159 |

In summary, the optimized PD1 blocking antibodies, P-1194, P-1201 and P-1238, built on a VH framework (IGHV3-23) that is of substantially lower sequence homology but superior biophysical properties, are able to fully or nearly fully retain antibody's functional activity and display improved similarity score to the closest human germline sequence, (IGHV3-23). The mutation details and similarity scores for each antibody are summarized in Table 14.

TABLE 14
CDR germlining, FR reversion mutations, and similarity scores to the closest
human germline sequences of exemplary optimized PD1 antibodies

| | | No. of FR | VH FR | VL Similarity | VH Similarity |
|---|---|---|---|---|---|
| | CDR germling | germlining | reversion | Score to | Score to |
| Ab ID | substitutions | substitutions | mutations | IGKV3D-11 | IGHV3-23 |
| P-0734 | N/A | N/A | N/A | 85.1% | 51.0% |
| P-1194 | ^{L}K27Q, ^{L}L54R, | 30 | ^{H}I69L, ^{H}R71T, | 87.2% | 83.7% |
| | ^{H}F59Y, ^{H}N60A, | | ^{H}N73S | | |
| | ^{H}E61D, ^{H}N65G | | | | |
| P-1201 | ^{L}K27Q, ^{L}L54R, | 30 | ^{H}I69L, ^{H}R71T, | 87.2% | 82.7% |
| | ^{H}N60A, ^{H}E61D, | | ^{H}N73S | | |
| | ^{H}N65G | | | | |
| P-1238 | ^{L}K27Q, ^{L}L54R, | 28 | ^{H}V48M, | 87.2% | 81.6% |
| | ^{H}F59Y, ^{H}N60A, | | ^{H}S49G, ^{H}I69L, | | |
| | ^{H}E61D, ^{H}N65G | | ^{H}R71T, ^{H}N73S | | |

### Example 6

### Germlining Substitutions Let to Decreased Hydrophobicity of the Optimized PD1 Blocking Antibodies

Among the 23 FDA and EMA approved therapeutic mAbs, pembrolizumab was the most hydrophobic one and consequently had the highest tendency to aggregate (Goyon et al., J. Chromatogr. B 1065-1066:35-43, 2017). Consistent with the experimentally-determined apparent hydrophobic interaction chromatography (HIC) retention factors (k), the SSH2.0 hydrophobicity prediction tool (http://i.uestc.edu.cn/SSH2/; Zhou et al., Front. Genet. 13:842127, 2022) indicated that both variable chains of pembrolizumab carry a significant risk of hydrophobic interaction. The probabilities of hydrophobic interaction for its VH and VL are 0.97 and 0.61, respectively. An antibody is predicted to have a high risk of hydrophobic interaction if the probability is 0.5 or more (with 1 being the maximum possible value).

While the focus of the germlining substitutions was to enhance the degree of “humanness” in the antibody sequence, the process also resulted in a significant reduction in the probabilities of hydrophobic interaction for multiple optimized antibody sequences. Table 15 provides a summary of the predicted probabilities of hydrophobic interaction for the variable domains of the exemplary optimized PD1 blocking antibody, as estimated by SSH2.0.

TABLE 15
Summary of exemplary antibodies with improved similarity
scores and reduced probabilities for hydrophobicity
interaction while retaining PD1 blocking activity

| | SEQ ID NOS | | Similarity score to | | Probability of | | PD1 |
|---|---|---|---|---|---|---|---|
| | Light | Heavy | human germline | | hydrophobic interaction | | blocking |
| PD1 Ab | chain | chain | VL | VH | VL | VH | activity |
| P-0734 | 42 | 43 | 85.1% | 79.6% | 0.607 | 0.971 | Reference |
| P-1174 | 44 | 45 | 87.2% | 83.7% | 0.131 | 0.848 | Fully retain |
| P-1271 | 44 | 49 | 87.2% | 88.8% | 0.131 | 0.185 | Fully retain |
| P-1194 | 44 | 46 | 87.2% | 83.7% | 0.131 | 0.794 | Almost fully |
| | | | | | | | retain |
| P-1201 | 44 | 47 | 87.2% | 82.7% | 0.131 | 0.794 | Almost fully |
| | | | | | | | retain |
| P-1238 | 44 | 48 | 87.2% | 81.6% | 0.131 | 0.740 | Fully retain |

As shown in Table 15, the two light chain CDR germlining substitutions, ^{L}K27Q and ^{L}L54R, markedly decreased the hydrophobicity probability of VL from 0.607 for P-0734 to 0.131. These two amino acid changes were applied to the VL in all the optimized PD1 blocking antibodies listed in Table 15. The CDR germline substitutions in the heavy chain only resulted in a marginal reduction in hydrophobicity, with hydrophobicity probabilities altering from 0.971 for (P-0734) to 0.848 for (P-1174) and to approximately 0.8 for antibodies with their VH based on VH-3 family frameworks. However, when germlining substitutions (^{H}V9A, ^{H}T76S, ^{H}K82aS, ^{H}Q83R, ^{H}F84S, ^{H}T108L) were implemented in the VH framework of P-1174, the resulting construct, P-1271, had a hydrophobicity probability of 0.185, much lower than that of P-1174.

Hydrophobic patches on an antibody's surface are often implicated as one of the main contributions to its propensity to aggregate. Furthermore, these hydrophobic patches can cause high viscosity. Consequently, the exemplary PD1 blocking antibodies, which have significantly diminished hydrophobic potentials are expected to exhibit improved biophysical properties. PD1-targeted IL-15 immunocytokine and VitoKine fusions constructed using these optimized PD1 blocking antibodies are also projected to have enhanced developability profiles.

### Example 7

### Design and Methods for In Vitro Activity Assessment of IL-15 Variants

Identifying IL-15 variants with optimally attenuated potency is crucial for constructing a PD1 targeted immunocytokine to achieve a balance between the cytokine and antibody components. In its native version, IL-15 exhibits significant disparities in potency and molecular weights when compared to antibodies. Additionally, when designing PD1 Ab-IL15 VitoKines, incorporating an IL-15 variant of specific potency facilitates a refined adjustment of both the inherent basal activity of the resultant VitoKine and its post-proteolytic function. Additionally, immunocytokine and VitoKine designs require IL-15 variants of different potency levels. All IL-15 variants were first produced as Fc fusions and their activity was assessed using functional assays.

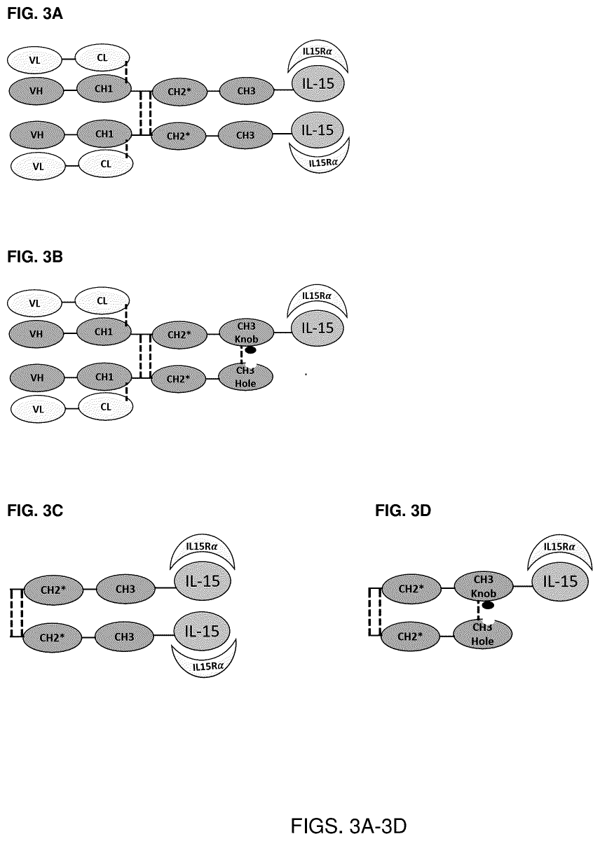

IL-15 variant Fc fusions were constructed by fusing a specific IL-15 variant to the C-terminus of an Fc chain (SEQ ID NO: 166), resulting in the dimeric IL-15 moiety. Alternatively, IL-15 variant was linked to the C-terminus of a knob chain from the knob-into-hole heterodimeric Fc chain pair (SEQ ID NOS: 167 and 168), yielding a monomeric IL-15 component. In both configurations, a flexible GS linker “GGGGSGGGGSGGGGS” (SEQ ID NO: 115) was employed, and an IL-15RαSushi+ domain (SEQ ID NO: 165) is non-covalently complexed with each IL-15 domain (as depicted in FIGS. 3C and 3D). Non-covalent complexation of the IL-15RαSushi+ domain was demonstrated to significantly enhance the developability of IL-15 fusion proteins in PCT/US2019/038210 application by the current inventors.

An established ex vivo human peripheral blood mononuclear cell (PBMC) assay was used to assess the functional activity of IL-15 variants. This was done to assess their ability to stimulate cell proliferation by measuring Ki67 expression in CD8 T cells and NK cells. Briefly, human PBMCs were isolated by Ficoll-Hypaque centrifugation from the buffy coat purchased from Blood Oklahoma Institute. Purified PBMCs were then treated with increasing doses of IL-15 variants and incubated at 37° C. for 5 days. On Day 5, cells were washed with FACS buffer (1% fetal bovine serum in phosphate buffered saline, pH 7.2) and first stained with an Fc-blocker (BioLegend), and surface marker antibodies, such as anti-human CD8-APC (BioLegend) and anti-human CD56-FITC, at a 1:50 dilution. Following a 30-minute incubation and a wash, the cells were treated with a fixation & permeabilization working solution (ThermoFisher) for another 30 minutes at room temperature in the dark. After centrifugation, cells were treated with permeabilization buffer (ThermoFisher) containing an anti-human Ki67-PE antibody (BD Life Sciences). After a final 30-minute incubation, cells were collected, washed, and resuspended in FACS buffer before being analyzed by the Attune NxT flow cytometer (Thermo Fisher). Data are expressed as a percentage of Ki67 positive cells in the gated population.

To determine the cross-reactivity IL-15 variants in mice using an in vitro method, the CTLL-2 cell proliferation assay was employed. CTLL-2, a cytotoxic murine T-cell line derived from a C57BL/6 mouse, is commonly used to assess the biological activity of IL-2 and IL-15 by measuring the extent of cell proliferation. Briefly, CTLL-2 cells in their logarithmic growth phase were washed three times with PBS buffer and resuspended at a density of 5×10⁵ cells/mL in the basal media (RPMI medium containing 10% fetal bovine serum, 2 mM L-glutamate, and 1 mM sodium pyruvate). After a 4-hour incubation at 37° C., 100 μL of these cells were added to serially diluted IL-15 fusion proteins in an equal volume of the basal media in each well of a 96-well plate, achieving a final density of 5×10⁴ cells/well. Following a 48-hour incubation at 37° C., cell viability was measured using the CellTiter-Glo® Luminescent Cell Viability Assay (from Promega), following the manufacturer's instructions. The results were analyzed using GraphPad Prism software to derive the EC₅₀ values.

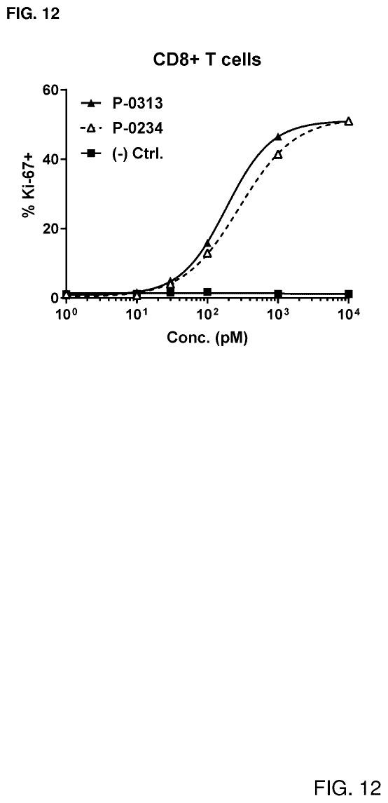

P-0234 and P-0313 are used interchangeably as the dimeric wild-type IL-15 control. While P-0313 contains the IL-15 S58D mutation (SEQ ID NO: 117), its activity is consistently comparable or slightly enhanced when compared to P-0234 (FIG. 12), which has the wild-type IL-15 (SEQ ID NO: 116). P-0217 is the monomeric equivalent of P-0234 and serves as the control for monomeric wild-type IL-15. Table 16 lists the exemplary IL-15 variants in Fc fusion constructs.

TABLE 16
Exemplary IL-15 variants in Fc fusion constructs

| Fc fusion | Fc SEQ | IL-15 SEQ | IL-15 | IL-15 |
|---|---|---|---|---|
| protein | ID NO(s): | ID NO: | substitutions | valency |
| P-0234 | 166 | 116 | wildtype | Dimeric |
| P-0217 | 167 + 168 | 116 | wildtype | Monomeric |
| P-0313 | 166 | 117 | S58D | Dimeric |
| P-0356 | 166 | 126 | I68F | Dimeric |
| P-0357 | 166 | 125 | I68D | Dimeric |
| P-0358 | 166 | 128 | I68H | Dimeric |
| P-0736 | 166 | 130 | I68Q | Dimeric |
| P-0737 | 166 | 127 | I68G | Dimeric |
| P-0738 | 166 | 129 | I68K | Dimeric |
| P-0769 | 166 | 123 | V63K | Dimeric |
| P-0770 | 166 | 124 | V63R | Dimeric |
| P-0771 | 166 | 121 | V63A | Dimeric |
| P-0768 | 166 | 131 | V63A/I68G | Dimeric |
| P-0772 | 166 | 133 | V63A/I68Q | Dimeric |
| P-0773 | 166 | 132 | V63A/I68H | Dimeric |
| P-0866 | 166 | 118 | 1-aa N-del. | Dimeric |
| P-0867 | 166 | 119 | 2-aa N-del. | Dimeric |
| P-0868 | 166 | 120 | 3-aa N-del | Dimeric |
| P-1059 | 166 | 142 | Q108N | Dimeric |
| P-1061 | 166 | 138 | Q108H | Dimeric |
| P-0684 | 167 + 168 | 136 | Q108E | Monomeric |
| P-0764 | 167 + 168 | 143 | Q108S | Monomeric |
| P-0793 | 167 + 168 | 134 | Q108A | Monomeric |
| P-0796 | 167 + 168 | 139 | Q108K | Monomeric |
| P-0836 | 167 + 168 | 141 | Q108M | Monomeric |
| P-1202 | 167 + 168 | 142 | Q108N | Monomeric |
| P-1203 | 167 + 168 | 144 | Q108T | Monomeric |
| P-1204 | 167 + 168 | 135 | Q108D | Monomeric |
| P-1205 | 167 + 168 | 137 | Q108F | Monomeric |
| P-1206 | 167 + 168 | 140 | Q108L | Monomeric |
| P-1207 | 167 + 168 | 145 | Q108Y | Monomeric |
| P-1293 | 167 + 168 | 160 | N112D | Monomeric |
| P-1324 | 167 + 168 | 161 | N112G | Monomeric |
| P-1294 | 167 + 168 | 162 | N112P | Monomeric |
| P-1325 | 167 + 168 | 163 | N112R | Monomeric |
| P-0832 | 167 + 168 | 149 | I68H/Q108M | Monomeric |
| P-0833 | 167 + 168 | 148 | I68F/Q108M | Monomeric |
| P-0834 | 167 + 168 | 146 | V63A/Q108M | Monomeric |
| P-0835 | 167 + 168 | 147 | V63K/Q108M | Monomeric |
| P-1242 | 167 + 168 | 151 | V63K/Q108N | Monomeric |
| P-1243 | 167 + 168 | 150 | I68H/Q108N | Monomeric |

### Example 8

### Attenuating IL-15 Activity by Substituting IL-15 Residues that Interact with IL-15Rβ

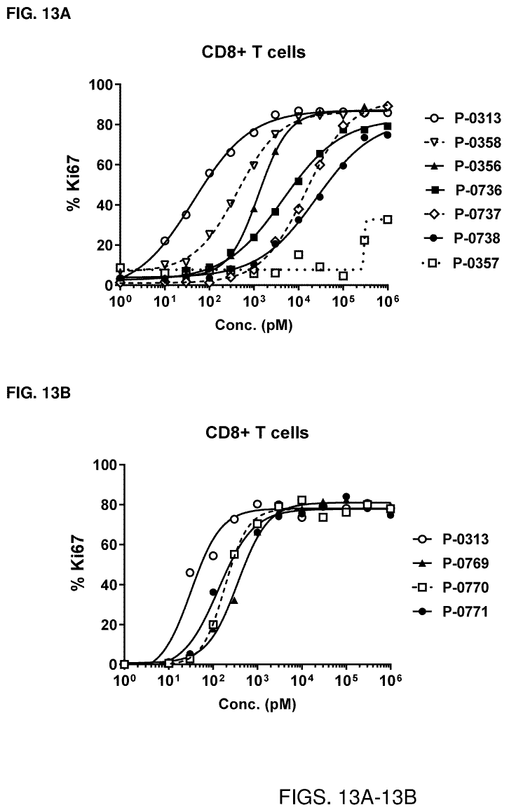

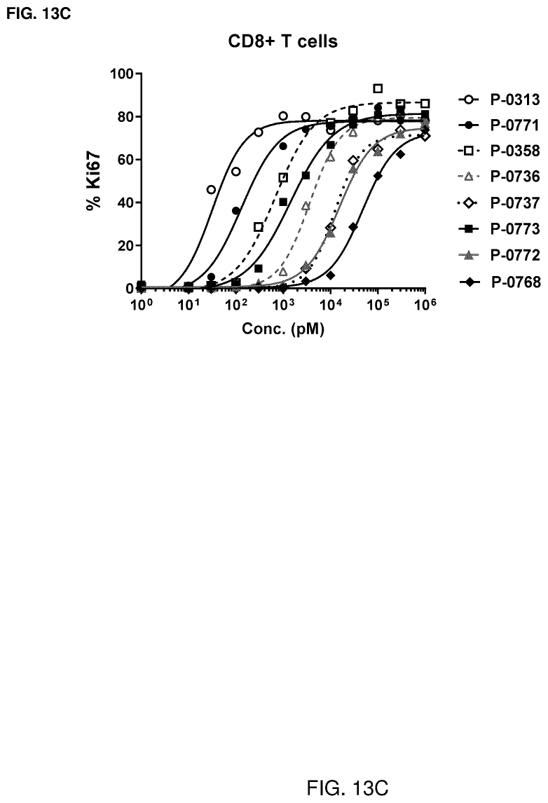

The selection of IL-15 mutations that disrupt IL-5Rβ interface was guided by inspecting the IL-15/IL-15R co-crystal structure (PDB ID: 4GS7 and Ring et al., 2012, Nat. Immunol. 13:1187-1195). While residue 168 forms only Van der Waals interaction with IL-15Rβ, the impact of its substitution is surprisingly diverse. Specifically, amino acid substitutions at position 168 yielded variants with a broad range of potency in stimulating CD8 T cell proliferation. The EC₅₀ values varied from 0.4 nM to approximately 300 nM, marking a significant difference of 700-fold. Compared to the control molecule, P-0313, the levels of activity reduction range from 10-fold to roughly 7000-fold. The results are illustrated in FIG. 13A and summarized in Table 17A.

TABLE 17A
Impact of exemplary I68 mutations on IL-15's
potency in stimulating CD8 T cell proliferation

| Fc | IL-15 | CD8 T cell Ki67 | Fold change |
|---|---|---|---|
| fusion | mutation | EC₅₀ (pM) | over P-0313 |
| P-0313 | S58D | 44 | 1 |
| P-0358 | I68H | 419 | 9.5 |
| P-0356 | I68F | 1251 | 29 |
| P-0736 | I68Q | 4473 | 102 |
| P-0737 | I68G | 14466 | 330 |
| P-0738 | I68K | 24842 | 564 |
| P-0357 | I68D | ~296000 | ~6727 |

In contrast to residue 168, V63 does not directly interact with IL-15Rβ residues, but it is in close proximity to important contact residues, D61 and N65, that form the receptor binding interface (refer to Ring et al., 2012, Nat. Immunol. 13:1187-1195). As expected, substitutions at the V63 position resulted in only moderate attenuation (approximately 4-10-fold reduction) of IL-15's ability to stimulate CD8 T cell proliferation (illustrated in FIG. 13B). EC₅₀ values of these variants in stimulating CD8 T cell proliferation, along with their fold reduction relative to P-0313 can be found in Table 17B.

TABLE 17B
Impact of exemplary V63 mutations on IL-15's
potency in promoting CD8 T cell proliferation

| Fc | IL-15 | CD8 T cell Ki67 | Fold change |
|---|---|---|---|
| fusion | mutation | EC₅₀ (pM) | over P-0313 |
| P-0313 | S58D | 32 | 1 |
| P-0739 | V63K | 359.8 | 11.3 |
| P-0740 | V63R | 184.4 | 5.8 |
| P-0741 | V63A | 131.7 | 4.2 |

While alterations in the amino acid at the V63 position led to only a modest reduction in IL-15's activity, it offers an approach to fine-tune the attenuation level by pairing it with other IL-15 amino acid changes that interfere IL-15 receptor interactions. Exemplary combination mutants incorporating V63A and one of the mutations at 168, including V68H, 168Q, and 168G, were constructed and their activity were assessed in human PBMCs. These combined mutations that disrupt IL-15Rβ interactions incrementally reduced the potency in stimulating CD8 T cell proliferation (detailed in Table 17C). When compared to P-0313, the V63A substitution brough about a 4.2-fold reduction in potency reduction. Consistently, within the mutational contexts of 168H, 168Q, and 168G, the V63A substitution resulted in a 2.1-fold, 4.3-fold, and 3.8-fold attenuation in CD8 T cell proliferation potency, respectively. The data are also illustrated in FIG. 13C.

TABLE 17C
Impact of the exemplary combined mutations that interfere
IL-15Rβ interactions on IL-15's Potency

| | | CD8 T cell | | Impact of |
|---|---|---|---|---|
| Fc | IL-15 | Ki67 EC₅₀ | Fold change | V63A |
| Fusion | mutation(s) | (pM) | over P-0313 | (fold change) |
| P-0313 | S58D | 31.7 | 1 | N/A |
| P-0771 | V63A | 132 | 4.2 | 4.2 |
| P-0358 | I68H | 683 | 21.5 | N/A |
| P-0773 | V63A/I68H | 1418 | 45 | 2.1 |
| P-0736 | I68Q | 3614 | 114 | N/A |
| P-0772 | V63A/I68Q | 15379 | 485 | 4.3 |
| P-0737 | I68G | 12783 | 403 | N/A |
| P-0768 | V63A/I68G | 48015 | 1513 | 3.8 |

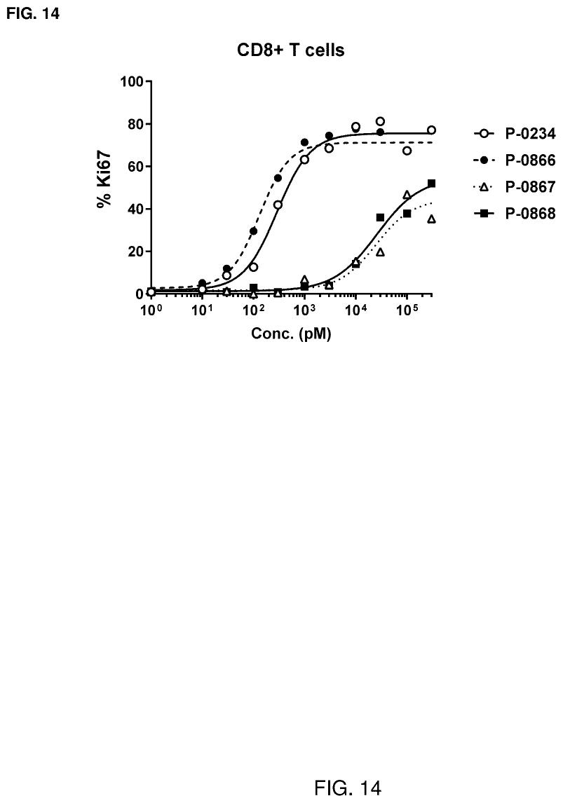

In addition to modifying IL-15 residues that interface with the IL-15Rβ receptor subunit for targeted activity attenuation, truncating the N-terminal residues presents an alternative strategy. The N-terminus of IL-15 is part of the alpha helix, which contains important residues, e.g., Ser7, Asp8 and Lys10, that engage with IL-15Rβ (Ring et al., 2012, Nat. Immunol. 13:1187-1195). Three dimeric IL-15 Fc fusion proteins, P-0866, P-0867, and P-0868, each with deletions of 1, 2, and 3 amino acids at the IL-15 N-terminus respectively, were assayed using the human PBMC assay. The results are depicted in FIG. 14. For P-0866, the deletion of a single amino acid did not affect its ability to stimulate CD8 T cell proliferation relative to the wild-type control, P-0234. Rather, a modest 2-fold increase in potency was noted (EC₅₀ of 130 pM compared to P-0234's EC₅₀ of 289 pM). Introducing a second amino acid deletion in P-0867 saw a significant drop in potency, nearly 100-fold reduction (EC₅₀ of 24,800 pM compared to 289 pM for P-0234). Adding one more deletion in P-0868 did not further diminish its functional activity.

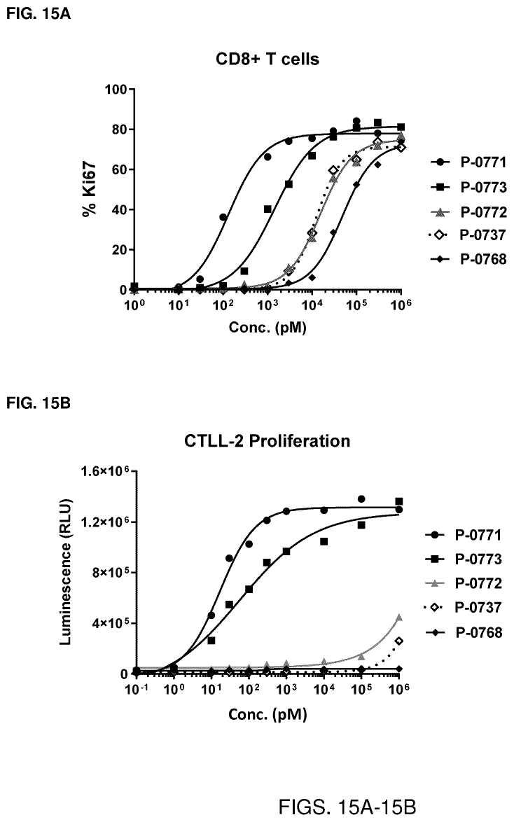

The CTLL-2 cell proliferation assay (described in Example 7) was subsequently used to assess the cross-reactivity IL-15 variants in mice. Specifically, five compounds—P-0771, P-0773, P-0772, P-0737, and P-0768—each with distinct IL-15Rβ-interfering mutations, were compared for their biological activity in terms of promoting human CD8 T cell proliferation and supporting mouse-derived CTLL-2 cell growth. The results were illustrated in FIGS. 15A and 15B and summarized in Table 18. From P-0771 to P-0773 to P-0772/P-0737, there were approximately 10-fold incremental decreases in the induction of Ki67 expression in human CD8 T cells. The potency gap between P-0771 and P-0768 was a significant 350-fold (with EC₅₀ values of 0.132 nM and 48 nM, respectively). In the CTLL-2 cell proliferation assay, P-0771 and P-0773 retained the biological activity observed in human PBMC assay, showing a 10-fold difference in EC₅₀ values. However, P-0772 and P-0737 exhibited a sharp decline in their ability to sustain CTLL-2 growth. This decline was not proportional to their potency in stimulating Ki67 expression in human CD8 T cells (˜5-log drop vs 100-fold reduction). Furthermore, P-0768 completely lost its capability to sustain CTLL-2 proliferation.

TABLE 18
Activity of IL-15 variants in stimulating human CD8 T cell proliferation
and sustaining mouse-derived CTLL-2 cell proliferation

| Fc | IL-15 | EC₅₀ (pM) | |
|---|---|---|---|
| Fusion | mutation(s) | CD8 T cell Ki67 | CTLL-2 proliferation |
| P-0771 | V63A | 132 | 32.7 |
| P-0773 | V63A/I68H | 1418 | 324 |
| P-0772 | V63A/I68Q | 15379 | Low |
| P-0737 | I68G | 12783 | Low |
| P-0768 | V63A/I68G | 48015 | Not detectable |

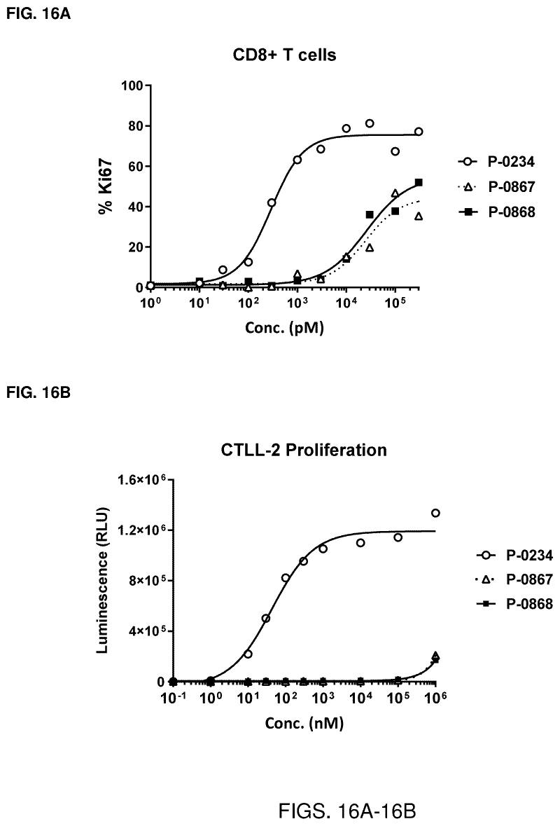

The disproportionate decrease in activity observed in murine cells compared to human cells was also seen in the two N-terminal deletion mutants, P-0867 and P-0868. As illustrated in FIGS. 16A, there was a nearly 100-fold reduction in stimulating CD8 T cell proliferation compared to the wild-type control, P-0234. However, these mutants nearly completely lost their effectiveness in supporting CTLL-2 cell growth, contrasted with the robust EC₅₀ of 32.7 nM by P-0234 (FIG. 16B).

To ensure that the absence of CTLL-2 activity correlates with the in vivo activity in mice, P-0768 was administered to naïve Balb/C mice to evaluate its impact on peripheral CD8 and NK cell proliferation. Consistent with the CTLL-2 assay results, there were no observable pharmacodynamic effects on peripheral lymphocytes, including CD8 T and NK cells. This was in sharp contrast to the pronounced expansion peripheral blood CD8 T and NK cells when either P-0313 or P-0773 was administered. Both these compounds exhibit CTLL2 activity that aligns with their activity in human cells activity, as detailed in Table 18.

The diminished or complete loss of cross-reactivity to mouse receptors makes it difficult to assess compounds with desired in vitro potency in human cells, such as P-0772 and P-0768, for in vivo pharmacodynamic effect and anti-tumor efficacy in well-established syngeneic mouse tumor models including CT26 and MC38. An alternative mutational strategy is needed.

### Example 9

### Modulating IL-15 Activity by Substituting IL-15 Residues that Interface with γc

IL-15's Q108 residue is one of the hotspots that interfaces with several key γc residues (Ring et al., 2012, Nat. Immunol. 13:1187-1195). Multiple IL-15 variants comprising amino acid substitution at Q108 were constructed and assessed for their impact on cell proliferation, specifically by measuring Ki67 expression in CD8 T cells of fresh human PBMCs. Exemplary fusion proteins of IL-15 variants containing mutations at the Q108 position can be found in Table 16.

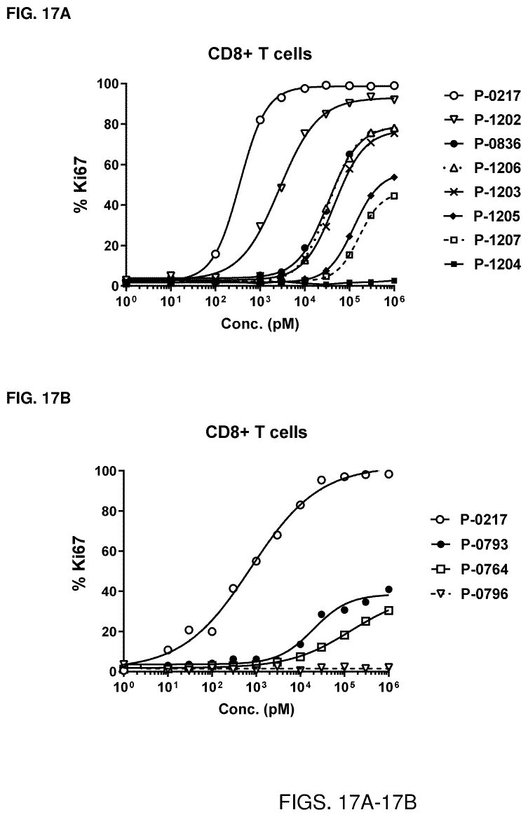

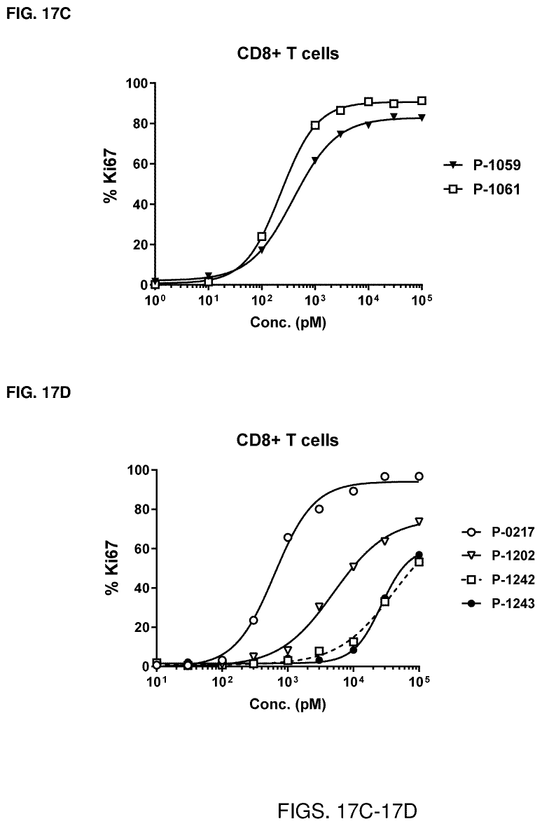

As illustrated in FIG. 17A, a spectrum of agonist activity in CD8 T cell proliferation arose from amino acid substitutions at the Q108 position of IL-15. This includes Q108M in P-0836, Q108N in P-1202, Q108T in P-1203, and Q108D in P-1204, Q108F in P-1205, Q108L in P-1206, and Q108Y in P-1207. The EC₅₀ values exhibit a wide range, from 2.7 nM for P-1202 (IL-15 Q108N) up to 164 nM for P-1207 (IL-15 Q108Y). Moreover, P-1204 (IL-15 Q108D) shows a complete loss of activity. These results are relative to the EC₅₀ value of 0.35 nM for P-0217, the wild-type counterpart.

FIGS. 17B and 17C further depict the ex vivo activity of additional IL-15 Q108 variants. P-0793 (IL-15 Q108A) and P-0764 (IL-15 Q108S) displayed a significant decrease in efficacy, showing EC₅₀ values ranging between 20-50 nM. Their signaling intensities were also remarkably lowered, with the signaling amplitudes down to 30-40% of the wild-type Eₘₐₓ (maximum possible effect), displaying partial agonist characteristics (FIG. 17B). Similar to P-1204 (IL-15 Q108D) and P-0684 (IL-15 Q108E; data not shown), P-0796 (IL-15 Q108K) completely lost its agonist capability as well. Shown in FIG. 17C is the comparison of P-1059 and P-1061, both Fc fusions comprising the dimeric IL-15 domain. The IL-15 Q108H mutation in P-1061 showed a slightly better potency than the Q108N mutation in P-1059 with EC₅₀ of 0.23 nM and 0.39 nM, respectively. A summary of the exemplary IL-15 variants with changes at the Q108 residue (consisting of a monomeric IL-15 unless otherwise noted) and their agonist potencies (EC₅₀ values in inducing CD8 T cell proliferation) can be found in Table 19.

TABLE 19
Impact of exemplary amino acid changes at
Q108 on CD8 T cell proliferation potency

| Fc | IL-15 | CD8 T cell Ki67 | Fold change |
|---|---|---|---|
| Fusion | mutations | EC₅₀ (nM) | over P-0217 |
| P-0217 | wildtype | 0.35 | 1 |
| P-1061 | Q108H^{#} | 0.39 | 1 |
| P-1202 | Q108N | 2.70 | 7.7 |
| P-0764 | Q108S | 19.6* | 56 |
| P-1206 | Q108L | 34.1 | 98 |
| P-0836 | Q108M | 33.7 | 97 |
| P-1203 | Q108T | 45.5 | 130 |
| P-0793 | Q108A | ~50* | ~140 |
| P-1205 | Q108F | 125 | 358 |
| P-1207 | Q108Y | 164 | 471 |
| P-0684 | Q108E | N/D | >1000 |
| P-0796 | Q108K | N/D | >1000 |
| P-1204 | Q108D | N/D | >1,000 |

*Displaying partial agonist characteristics with lower signaling amplitudes
^{#}Dimeric IL-15

In summary, IL-15 mutations at the Q108 residue can be divided into distinct categories, based the extent to which the substitution impacts the potency of CD8 T cell proliferation. Mutations in Category 1, exemplified by Q108H and Q108N, cause only slight to modest reductions in IL-15 agonist activity. Mutations in Category 2, represented by Q108A, Q108L, Q108M, Q108S, and Q108T, involve amino acids with either non-aromatic hydrophobic side chains or polar noncharged side chains, leading to a substantial decrease in activity. In Category 3, mutants exemplified by Q108F and Q108Y involve aromatic amino acid substitutions and display a more drastic activity drop compared to the Category 2 mutations. Category 4 mutants, exemplified by Q108D, D108E, and Q108K, involve charged amino acid replacements and display complete activity abrogation. Within each Category, mutations with similar characteristics are expected to demonstrate comparable activity levels. For example, IL-15 variants with Q1081 or Q108V mutations (residues with non-aromatic hydrophobic side chains), are predicted to display similar activity to other variants in Category 2.

The potency of IL-15 can be further tuned by pairing with other mutations that interfere with receptor interactions. For instance, combination mutants that integrate modifications affecting the IL-15Rβ interaction, such as V63A, V63K, V68H, and V68F, with changes impacting the γc interaction, like Q108N and Q108M, have been constructed. When tested for CD8 T cell proliferation in human PBMCs, these combined substitutions demonstrated an incremental reduction in IL-15 agonist activity. This notion was exemplified by comparing the activity of P-1242 and P-1243 to that of P-1202 in stimulating CD8 T cell proliferation (FIG. 17D). All these IL-15 variants contain the Q108N mutation, but P-1242 adds the V63K mutation, while P-1243 incorporates 168H. The EC₅₀ values for P-1242 and P-1243 were 41 nM and 27 nM, respectively compared to 5.0 nM for P-1202, indicating a 5-8-fold reduction due to the additional amino acid changes, consistent with the impact observed for these two mutations (refer to Table 17).

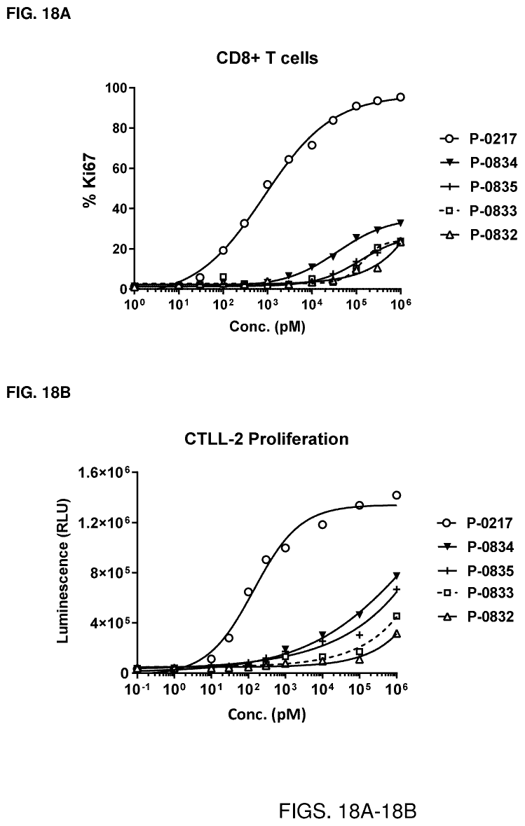

Further, IL-15 variants with Q108 mutations retained mouse cross-reactivity, despite substantially reduced agonist activity for human lymphocytes. This contrasted sharply with 168 variants like 168Q and 168G, which lost this cross-reactivity (FIGS. 14A and 14B). In FIG. 18, four IL-15 variants, all with the Q108M mutation plus one additional substitution that interferes IL-15Rβ interaction, including 168H in P-0832, 168F in P-0833, V63A in P-0834, and V63K in P-0835, showed a significant reduction in efficacy and signaling strength in stimulating CD8 T cell proliferation compared to the wild-type control, P-0217. The variations in activity among them were ascribed to distinct IL-15Rβ-disrupting substitutions (FIG. 18A). Such activity pattern was consistent across CTLL-2 assays (FIG. 18B), suggesting that the Q108 mutated IL-15 variants maintain their ability to cross-react with mouse receptors. The preservation of mouse cross-reactivity streamlines translational research, allowing convenient mouse studies to investigate in vivo pharmacodynamics and anti-tumor efficacy, particularly for IL-15 variants with targeted low potency.

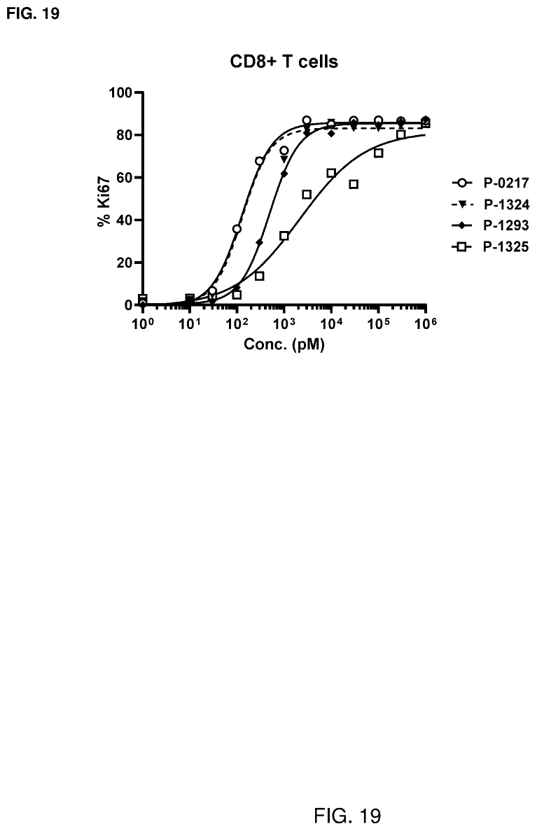

In addition to Q108, other IL-15 residues at or around γc receptor interface, including but not limited to D30, H32, M109, and N112, can also be modified to achieve various levels of potency attenuation. Exemplary substitutions include D30T, H32E, H32D, H32N, H32Q, M109A, M109H, M109R, N112D, N112R, N112G, and N112P. These amino acid alterations resulted in varying but generally modest decreases in the activity. For example, FIG. 19, P-1324 (N112G) had no change in EC₅₀ value for CD8 T cell proliferation. Meanwhile, P-1293 (N112D) mutation showed a 4-fold reduction (EC₅₀ value of 0.49 nM compared to 0.12 nM for P-0217), and P-135 (N112P) exhibited an 18-fold reduction in the EC₅₀ value (2.14 nM versus 0.12 nM). Furthermore, these mutations can be paired with other IL-15Rβ and/or γc-interfering mutations to achieve specific potency levels. As can be appreciated by skilled artisan, any additional combination mutants come with the spirit and scope of the present invention.

In summary, integrating of amino acid deletions, IL-15Rβ-interfering substitutions, or modifications that disrupt γc into IL-15, whether individually or in combination, led to variants displaying a broad range of potency levels in stimulating human cytotoxic lymphocytes. Notably, when the potency falls below a certain threshold, N-terminal deletions or certain substitutions impacting IL-15's interaction with IL-15Rβ can lead to a loss of mouse receptor cross-reactivity. Yet, with similar potency levels, changes that interfere with γc preserve IL-15's ability to cross-react with mice.

### Example 10

### Constructing PD1 Ab-IL-15 Immunocytokines with Optimized PD1 Antibodies and IL-15 Variants Across Different Potency Levels

Tethering an IL-15 variant to an PD1 antibody aims to deliver the IL-15 variant preferentially in cis to PD1+ cells, such as activated and exhausted CD8+T in tumor microenvironment, facilitating selective signaling. This strategy also reduces systemic exposure of IL-15 and can provide synergy by removing the negative regulation and reinvigorating T cells in both function and number. Using IL-15 variants with attenuated potency helps balance the disparity in potency and molecular weights between the cytokine and antibody arms in their native forms. This balance allows for optimal dosing and preserves function of each arm. Diminished cytokine activity is expected to minimize peripheral activation, mitigate antigen-sink and target-mediated deposition in vivo, and promote tumor targeting via the antibody arm.

The PD1 antibodies used to construct PD1 Ab-IL-15 immunocytokines were selected from the optimized human PD1 blocking antibodies comprising light chain sequences set forth in SEQ ID NO: 44 and heavy chain sequences set forth in SEQ ID NOS: 45-49. These optimized PD1 blocking antibodies have a high affinity for the human PD1 protein and demonstrate equal or comparable potency as pembrolizumab in blocking PD1. They also possess a higher sequence similarity score to the closest human germline sequence, resulting in an improved degree of humanness compared to pembrolizumab. Furthermore, they are predicted to have lower hydrophobicity, which in turn is likely to lower their aggregation propensity than pembrolizumab. PD1-targeted IL-15 immunocytokines constructed using these optimized PD1 blocking antibodies are also projected to have enhanced developability profiles.

With a flexible linker, ‘GGGGSGGGGSGGGGS’ (SEQ ID NO: 115), the IL-15 domain was fused either to the C-terminus of a PD1 blocking antibody heavy chain forming a dimeric IL-15 structure (illustrated in FIG. 3A) or to the C-terminus of the knob chain of a knob-into-hole heterodimeric heavy chain pair resulting in a monomeric IL-15 structure (shown in FIG. 3A). In both configurations, the IL-15RαSushi+ domain (SEQ ID NO: 165) complexed non-covalently with IL-15 domain through co-expression during cell culture.

As can be appreciated by skilled artisan, any IL-15 variants, exhibiting diverse potency levels as disclosed in current invention, including but not limited to sequences set forth in SEQ ID NOS: 117-163, can serve as the building block in constructing PD1 Ab-IL-15 immunocytokines to potentiate/augment PD1 antibody-based therapies for various cancers. Additionally, adopting a monomeric IL-15 in these constructs is expected to offer an additional approach to modulate IL-15 potency, circumventing the avidity effect. Table 20A lists the exemplary PD1 Ab-IL-15 immunocytokine constructs.

TABLE 20A
Exemplary human PD1 Ab-IL-15 immunocytokine constructs

| IL-15 | Immunocytokine | IL-15 | IL-15 |
|---|---|---|---|
| Immunocytokine | SEQ ID NOs: | mutations | Valency |
| P-0870 | 175 + 50 + 165 | V63A/I68H | Dimeric |
| P-0886 | 176 + 50 + 165 | 2-aa N-del. | Dimeric |
| P-1369 | 177 + 44 + 165 | V63A/I68H | Dimeric |
| P-1385 | 178 + 174 + 44 + 165 | V63A/I68H | Monomeric |
| P-1380 | 179 + 174 + 44 + 165 | Q108N | Monomeric |
| P-1352 | 180 + 174 + 44 + 165 | I68H/Q108N | Monomeric |

Given that the human PD1 blocking antibodies of the present invention did not bind to mouse PD1, surrogate mouse PD1 Ab-IL-15 immunocytokines in either dimeric or monomeric forms were produced analogously. The linker connecting the PD1 antibody heavy chain and IL-15 domain is set forth in SEQ ID NO: 115. In both configurations, an IL-15RαSushi+ domain (SEQ ID NO: 165) is non-covalently complexed with each IL-15 domain (as depicted in FIGS. 3A and 3B). These surrogate immunocytokines were used for in vivo studies to assess their pharmacodynamic effects on lymphocyte stimulation in mice and anti-tumor efficacy in syngeneic mouse tumor models. Table 20B lists the detailed information of the surrogate constructs.

TABLE 20B
Exemplary Surrogate mouse PD1 Ab-
IL-15 immunocytokine constructs

| | | IL-15 | | |
|---|---|---|---|---|
| | Component | domain | | |
| IL-15 | PD1 Ab | SEQ ID | IL-15 | IL-15 |
| Immunocytokine | SEQ ID NOS: | Nos: | mutations | Valency |
| P-1284 | 169 + 170 + 52 | 116 | Wild-type | Monomeric |
| P-0869 | 52 + 53 | 132 | V63A/I68H | Dimeric |
| P-1266 | 169 + 170 + 52 | 132 | V63A/I68H | Monomeric |
| P-1295 | 169 + 170 + 52 | 142 | Q108N | Monomeric |
| P-1296 | 169 + 170 + 52 | 151 | I68H/Q108N | Monomeric |

Construction of the expression vectors, transient expression, as well as the subsequent purification and characterization of these immunocytokine fusions were carried out based on the procedures outlined in Example 2.

### Example 11

### Ex Vivo Characterization of PD1 Ab-IL-15 Immunocytokines

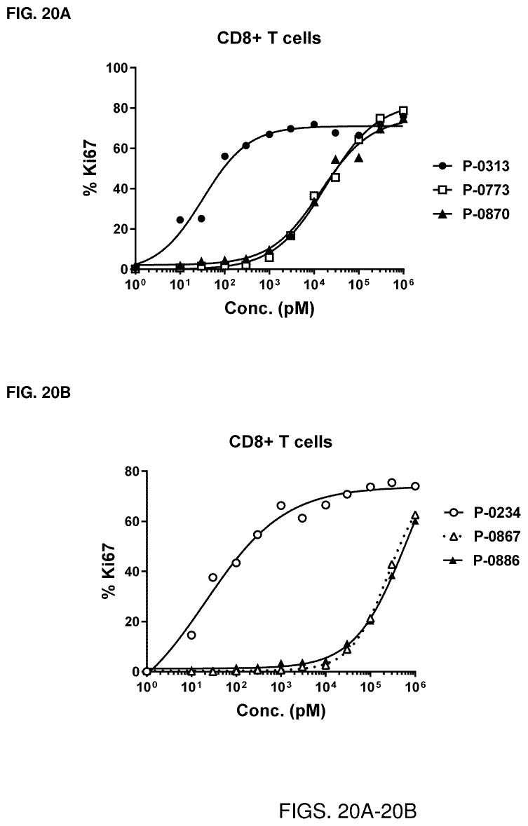

It is important to demonstrate that the observed potency levels of the IL-15 variants when in the Fc fusion format remain consistent when fused with an antibody. This consistency ensures the reliability of the results across different fusion formats. As depicted in FIG. 20, whether the IL-15 domain was fused to an Fc or a PD1 blocking antibody, its biological activity remains consistent. Specifically, P0773 is an Fc fusion and P-0870 is a PD1 antibody fusion, both containing the dimeric IL-15 variant V63A/168H, and they exhibited identical potency in stimulating dose-dependent increases in Ki67 expression in CD8 T cells (FIG. 20A). P-0867 and P-0886 in FIG. 20B are a similar pair, both featuring a 2-amino acid deletion at the N-terminus of IL-15 and displayed the same activity. This consistency across formats underscores the reliability and relevance the observed potency levels of IL-15 variants.

It is also essential that the PD1 antibody retains its binding and functional activities when incorporated into the PD1 Ab-IL-15 immunocytokines. PD1 antibodies of superior target-binding and PD1 blocking function can enhance the specificity and selectivity of TIL-targeting, and further synergize with IL-15 anticancer immune response by efficiently reversing T cell anergy and exhaustion.

Two ELISA assays were conducted to evaluate the PD1 binding and inhibition capabilities of the exemplary immunocytokine P-1352 against its component PD1 antibody, P-1271. A non-targeting germline antibody, P-1260 (SEQ ID NOS: 171, 172 and 173) was included as the negative control. The binding ELISA procedures were outlined in Example 3. The competition ELISA followed a similar protocol. After the coating, blocking, and washing steps, each well received a mixture of biotinylated human PD-L1-Fc (Acro Biosystems) at 0.5 μg/mL with an equal volume of 3-fold serial dilutions of either P-1271 or P-1352, starting at a concentration of 100 μM. After 1-hour incubation at 37° C., HRP-conjugated Streptavidin (ThermoFisher) diluted to 0.1 μg/ml were added to the plate and incubate at 37° C. for 1 hour. The plate was subsequently developed using TMB (ThermoFisher) substrate. Absorbance readings were taken at 450 nm, with 630 nm as the reference wavelength. The half-maximal inhibitory concentrations (IC50) values were deduced using GraphPad Prism software.

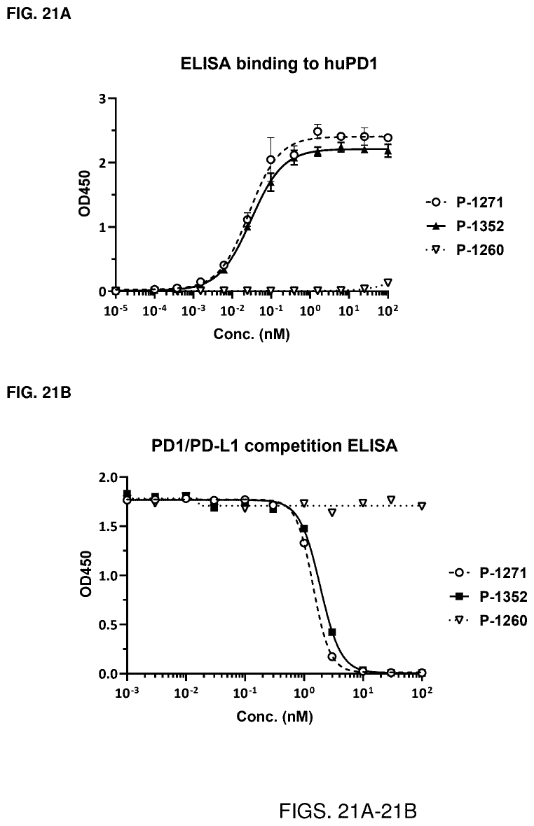

FIG. 21A shows that both P-1271 and P-1352 exhibit undistinguishable PD1 binding capabilities, demonstrated by their EC₅₀ values of 26.8 pM and 30.4 pM, respectively. Similarly, FIG. 21B reveals their consistent efficiency in blocking PD-L1 from binding to PD1 with equal potency with IC50 values of 1.42 nM for P-1271 and 1.88 nM for P-1352. These findings underscore that the PD1 antibody fully retained its binding and blocking activity when incorporated into immunocytokine constructs.

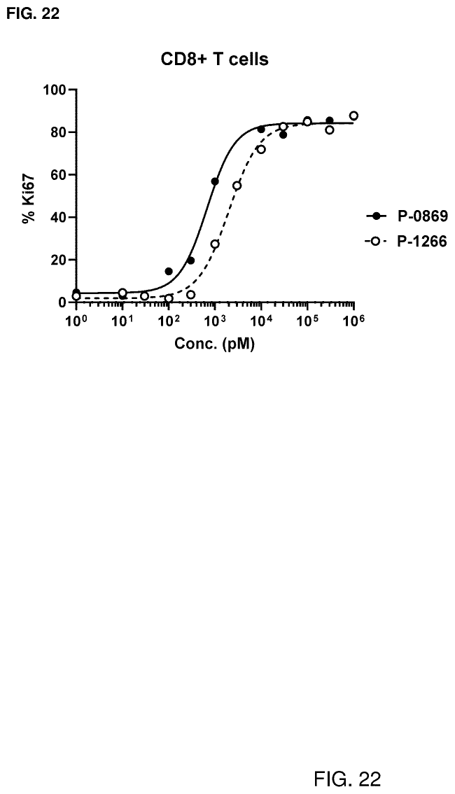

Additionally, FIG. 22 highlights that transitioning the IL-15 from its dimeric form in P-0869 to its monomeric form in P-1266 resulted in a 3-fold decrease in ex vivo activity, with EC₅₀ values of 0.67 nM and 2.0 nM for P-9869 and P-1266, respectively. Both P-0869 and P-1266 are PD1 Ab-IL-15 immunocytokines that incorporate the IL-15 V63A/168H variant. These findings underscore that incorporating a monomeric IL-15 in fusion constructs could offer an alternative approach to modulate IL-15 potency, circumventing the avidity effect commonly observed with dimeric forms.

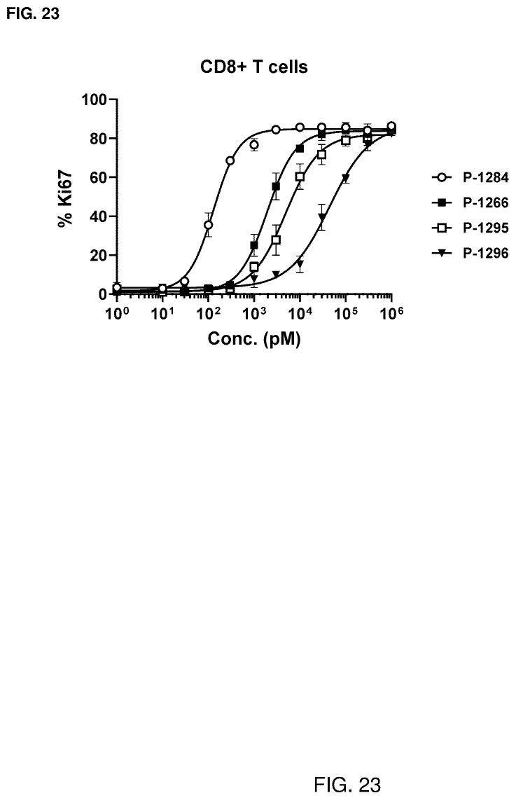

Finally, the mouse PD1 Ab-IL-15 immunocytokines, P-1266, P-1295, and P-1296, were assessed for their activity in stimulating Ki67 expression in human CD8+T before proceeding with in vivo studies. The monomeric IL-15 variants in these immunocytokines have mutations including V63A/168H (targeting IL-15Rβ binding), Q108N (targeting γc binding), and 168H/Q108N (interfering both IL-15Rβγ binding). As illustrated in FIG. 23, the potency, indicated by EC₅₀ values, is 1.94 nM for P-1266, 4.93 nM for P-1295, and 44.3 nM for P-1296. When compared to the EC₅₀ value of 0.13 nM for P-1284, the wild-type IL-15 control, the decrease in potency is by factors of 15, 38, and 340, respectively.

### Example 12

### Pharmacokinetic and Pharmacodynamic Effects of PD1 Ab-IL-15 Immunocytokines in Mice

The pharmacodynamic effects of PD1Ab-IL-15 immunocytokines on immune cells in peripheral blood of C57BL/6 mice were investigated using P-1266 and P-1295. The monomeric IL-15 domain in P-1266, containing the V63A/168H mutations, demonstrated a potency that was 2-3 times higher than that of P-1295 (with the Q108N mutation) in stimulating Ki67 expression in human CD8 T cells (FIG. 23).

Seven-week-old female C57BL/6 mice were received from Charles River Laboratory and acclimated in-house prior to the study. On Day 0, the mice were intraperitoneally administered with either a Vehicle (sterile PBS buffer) or a single dose of each test compound, P-1266 and P-1295, at a dosage of 1.5 mg/kg (mpk). Each group consisted of five mice. Blood samples were taken on Days 0, 5, 7, 10, and 12 following the injection and subsequently processed to prepare single cell suspensions.

In brief, red blood cell were lysed using BD Pharmingen lysis buffe and the total number of viable mononuclear blood cells were counted excluding the dead cells by trypan blue. These lysed immune cells underwent a fixation and permeabilization process by being incubated for 30 minutes at room temperature in the dak using a fixation/permeabilization buffer (eBioscience). After washing, the fixed and permeabilized cells were stained with antibodies to identify different immune cell subsets using a flow cytometer (Beckton Dickinson). Additionally, the Ki67 proliferation marker and Granzyme B cytotoxic marker were used to assess cell proliferation and activation within the identified subsets. Different immune cell subsets were identified, and the absolute numbers of circulating cells were quantified on a flow cytometer using the following commercially available antibodies: CD3-APC.Cy7, CD8-Percp-cy5.5, CD335-APC, Ki67-PE, and granzyme B-BV421. Data from flow cytometry was analyzed using FlowJo software and results were plotted with GraphPad Prism.

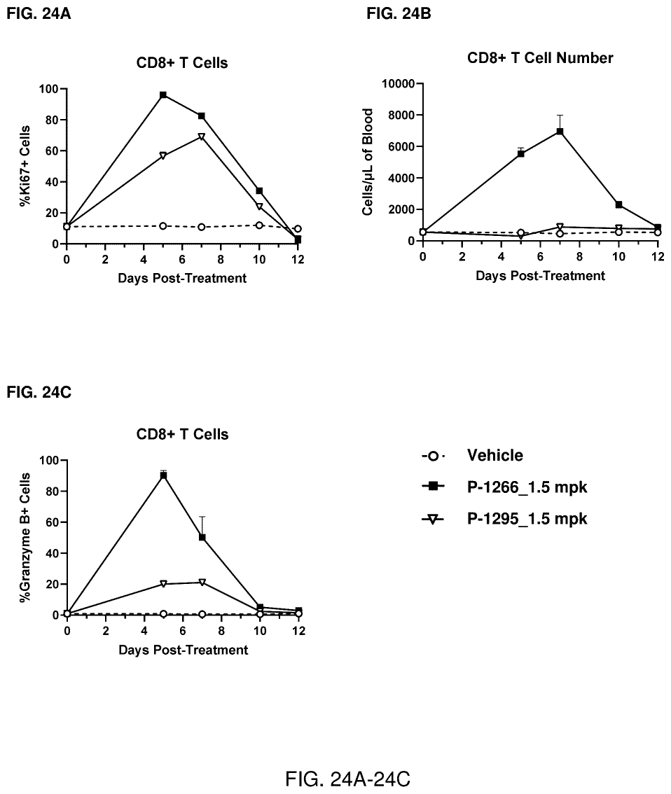

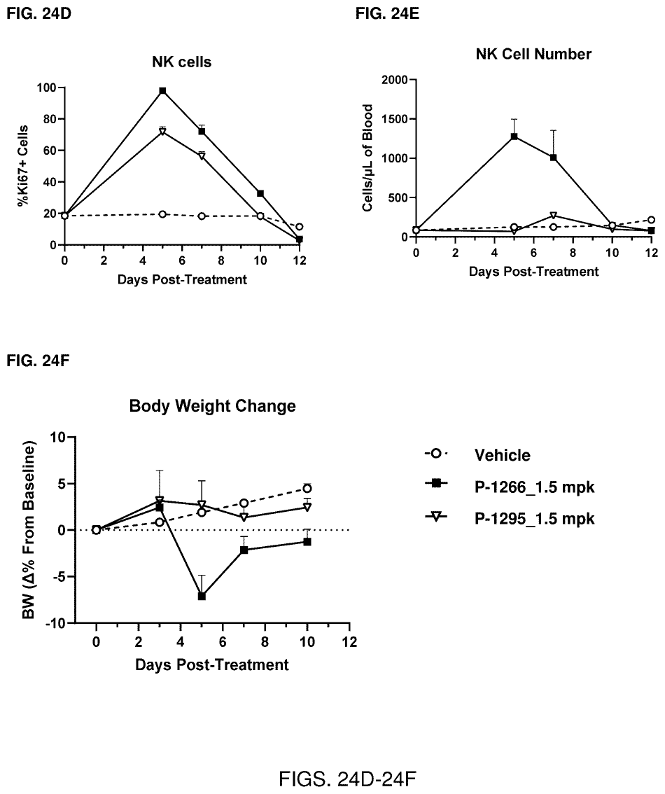

Both P-1266 and P-1295 stimulated a marked increase in the percentage of cells expressing Ki67, a marker indicating cell proliferation, in CD8+T (FIG. 24A) and NK Cells (FIG. 24D). The slight difference in the maximal signals aligns with the difference in their in vitro activities, suggesting that both compounds exhibit similar cross-reactivity to mouse receptors. For NK cells, known to be more responsive to IL-15, both showed peak activity on Day 5; for CD8 cells, P-1266 reached its peak on Day 5 while P-1295 peaked on Day 7, consistent with their respective potency.

In contrast to cell proliferation, there were significant differences in the cell expansion of CD8 T cells (FIG. 24B) and NK cells (FIG. 24E) between P-1266 and P-1295. Responding to P-1266, CD8 T cells increased from 550 to 6955 cells/μL of blood at the peak on Day 7, an increase of 12.5 times. Similarly, NK cells underwent a 15-fold surge, reaching a peak of 1275 cells/μL of blood on Day 5 from an initial count of 86 cells/μL. On the other hand, P-1295 led to only a slight 1.6-fold rise in CD8 T cells and a modest 3-fold growth in NK cells. This pattern is reflected in the percentage of CD8 T cells expressing the activation marker, granzyme B. For P-1295, it is significantly lower at 21% compared to 90% for P-1266 (FIG. 24C). Given that the disparity in mouse receptor cross-reactivity isn't the likely cause, it's plausible that the variation in cell expansion results from that the Q108N mutation in P-1295 disrupting its interaction with γc, while the V63A/168H mutations in P-1266 affects its bond with IL-15Rβ. This in turn impacts how each receptor influences the signaling cascade leading to cell expansion.

Moreover, P-1266 was associated with a noticeable drop in body weight on Day 5, which aligns with the dramatic expansion of cytotoxic lymphocytes. In contrast, P-1295 exhibited no body weight loss (FIG. 24F).

P-1266 and P-1295's pharmacokinetic (PK) effects were compared in a concurrently conducted in vivo study. Each compound was administered by intravenous injection at a dose of 1 mg/kg, Blood samples were withdrawn at 4 hours, 24 hours, 48 hours, 72 hours, 120 hours, 168 hours, and 240 hours post-injection by cheek bleeding. Each group consisted of 3 mice, and blood was taken either weekly or every three days, with a maximum frequency of twice per mouse.

The serum concentrations of the compounds were determined using an ELISA assay. Briefly, maxisorp plates were coated with mouse PD1 protein (R&D systems) overnight at 4° C. Following this, plates were blocked with Superblock (ThermoFisher). Blood samples at various dilutions were added to the plates and incubated for one hour at room temperature. A biotinylated monoclonal anti-IL 15 antibody (BD Bioscience) was applied, paired with HRP-conjugated streptavidin (ThermoFisher). The resultant signals were developed using the Ultra TMB substrate solution, and values were extrapolated from non-linear regression curve fits in GraphPad Prism.

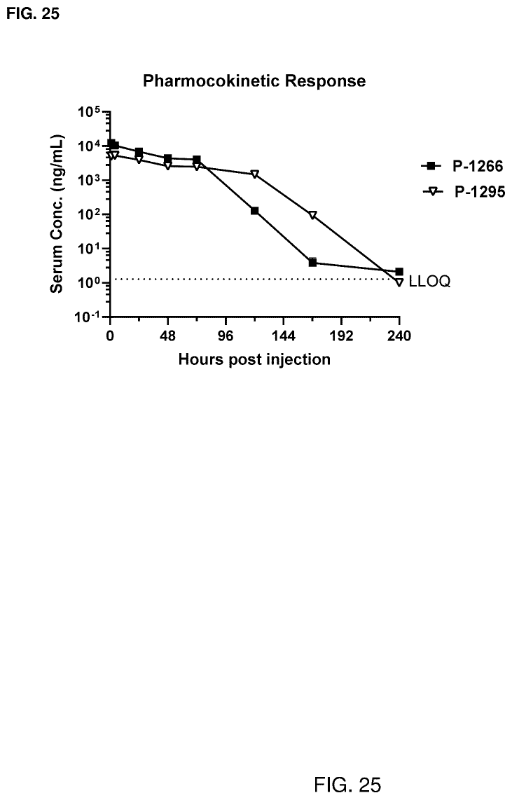

As shown in FIG. 25, P-1295 exhibits improved PK profile compared to P-1266. P-1295's serum concentration remained constant up to 128 hours after a 1 mg/kg dosage, while P-1266's concentration began to decrease by 72 hours. For both compounds, serum concentrations approached the lower limit of quantification (LLOQ), represented by the dotted line. The improved PK profile of P-1295 could be attributed to its diminished lymphocyte expansion, stemming from its γc-interfering mutation. This potentially leads to reduced target-mediated drug deposition and target sink.

Further, in a parallel pharmacodynamic experiment, we compared the effects of P-1266 and P-1296 on peripheral lymphocytes in MC38 tumor-bearing mice (with tumor model specifics detailed in Example 13). The treatments consisted of a Vehicle control, P-1266 at a dose of 1.5 mg/kg, and P-1296 at doses of 1.5 and 3 mg/kg, with each group having four mice. Five days post-injection, blood samples were collected and subsequently processed to prepare single cell suspensions. Given the observed activity pattern of P-1295 and the fact that P-1296, with its significantly reduced potency, contains the same γc-interfering mutation Q108N as in P-1295 alongside the 168H mutation targeting IL-15Rβ interaction, it was hypothesized that P-1296 would stimulate lower Ki67 expression on lymphocytes. Moreover, a more pronounced diminishment in cell expansion activity was anticipated.

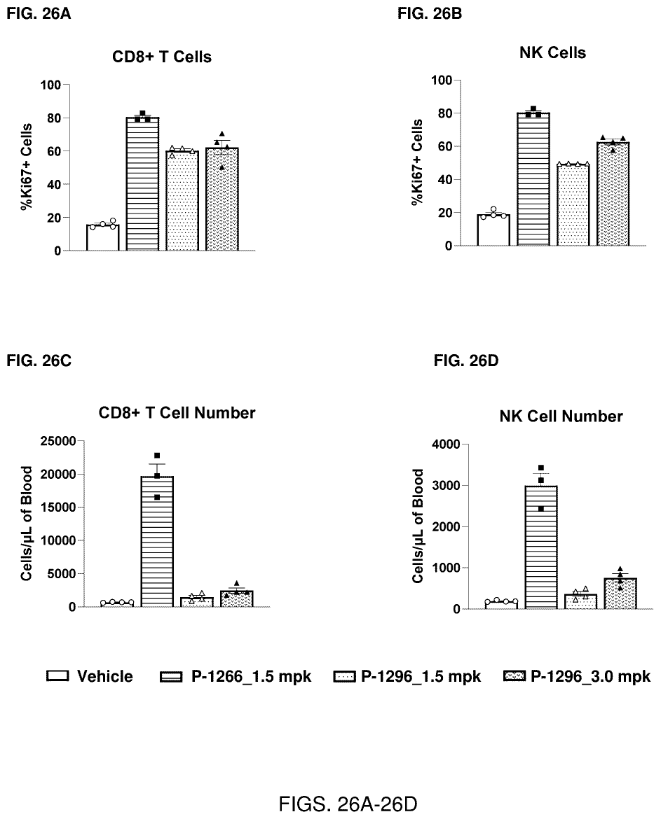

As illustrated in FIG. 26, when P-1296 was administered at dosages of 1.5 and 3 mg/kg, it did stimulate notable Ki67 expression on CD8 T (shown in FIG. 26A) and NK cells (FIG. 26B), when compared to the Vehicle control. However, the expression was still less intense than that seen with P-1266. This data confirms the idea that P-1296, even with its diminished potency, still reacts with mouse receptors. A trend echoing P-1295's results (FIG. 24) was seen with P-1296, where the expansion of both lymphocytes was significantly lower than what was observed with P-1266, as illustrated in FIGS. 26C and 26D.

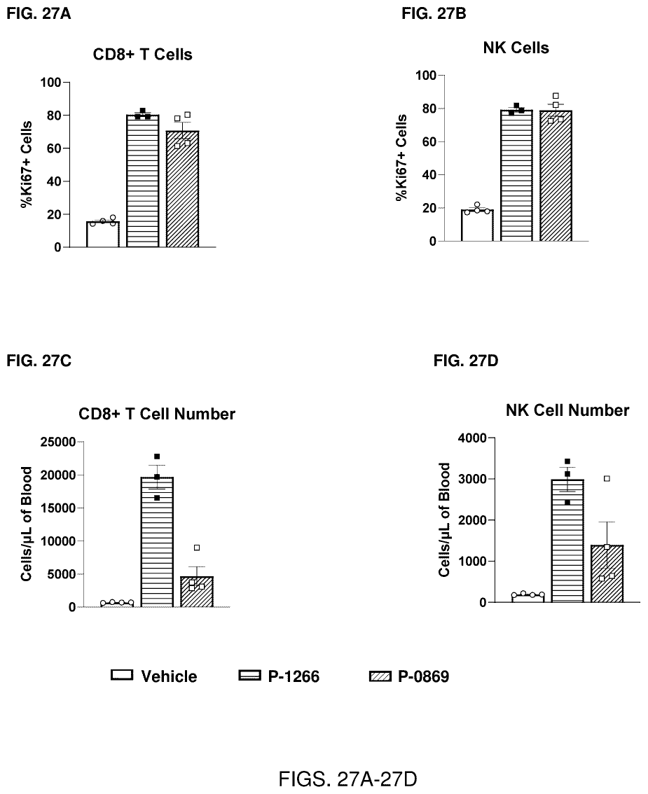

It was generally believed that incorporating a monomeric IL-15 domain can circumvent the dimeric form's avidity effect, leading to lower potency. This was evidenced by a 3-fold decrease in activity when transitioning the IL-15 from its dimeric form in P-0869 to its monomeric form in P-1266 (FIG. 22). However, when these compounds were tested in MC38 tumor-bearing mice at a dose of 1.5 mg/kg, the immunophenotyping outcome assessed 5 days later defied this general assumption and the in vivo results. Notably, while the dimeric P-0869 led to comparable Ki67 expression to its monomeric counterpart, P-1266, it unexpectedly prompted a substantially reduced cell expansion, as illustrated in FIG. 27. Specifically, P-1266 treatment led to a 26-fold surge in CD8 T cells and 17-fold boost in NK cells, while P-0869 yielded just a 6-fold and 8-fold increase in CD8 T and NK cells, respectively.

In summary, when compared with the IL-15 variant impacting IL-15Rβ binding, the IL-15 variant with γc-interfering mutation trigger Ki67 expression in mice that aligns with its ex vivo potency, yet the increase in cell count is disproportionately lower, which is accompanied with an improved PK profile. Furthermore, the dimeric IL-15 triggered far less lymphocyte expansion than its monomeric equivalent, a finding that contradicts both widespread perception and in vitro observations.

### Example 13

### Anti-Tumor Efficacy of PD1 Ab-IL-15 Immunocytokines in Syngeneic Mouse Tumor Models

The anti-tumor efficacy of PD1 Ab-IL-15 immunocytokine was investigated in both the MC38 and CT26 mouse colon carcinoma models. Both tumor models have been extensively used and proved to be highly valuable for assessing the efficacy of anti-cancer immunotherapies. Notably, CT26 is considered as a “cold” tumor” and is less responsive than MC38 model to PD1 therapy.

For the MC38 tumors, female C57BL/6 mice aged between 7-9 weeks were implanted subcutaneously on the right flank with 5×10⁵ MC38 colon carcinoma cells. About two weeks later, mice with established MC38 tumors, having an average volume approximately 75 mm³, were randomized into groups, marking that day as Day 0. In a parallel manner, CT26 tumor model was established by implanting 5×10⁵ CT26 cells subcutaneously on the right flank of female Balb/C mice. After between 9-11 days, once the average tumor volume reached approximately 75 mm³, the mice were grouped, designating that day as Day 0. Test compounds were dosed via intraperitoneal injections on Day 1, the next day following randomization. Tumor size and body weight were checked bi-weekly. The tumor volume (TV) was monitored using caliper and calculated as: volume=0.5×(width)²×(length). The tumor growth inhibition (TGI, %) was calculated using the following formula: TGI (%)=[1−(TV of the treated group)/(TV of the control group)]×100(%). The termination criterion for sacrificing animals was when the tumor size reached/exceeded 1500 mm³ and/or tumor became necrotic.

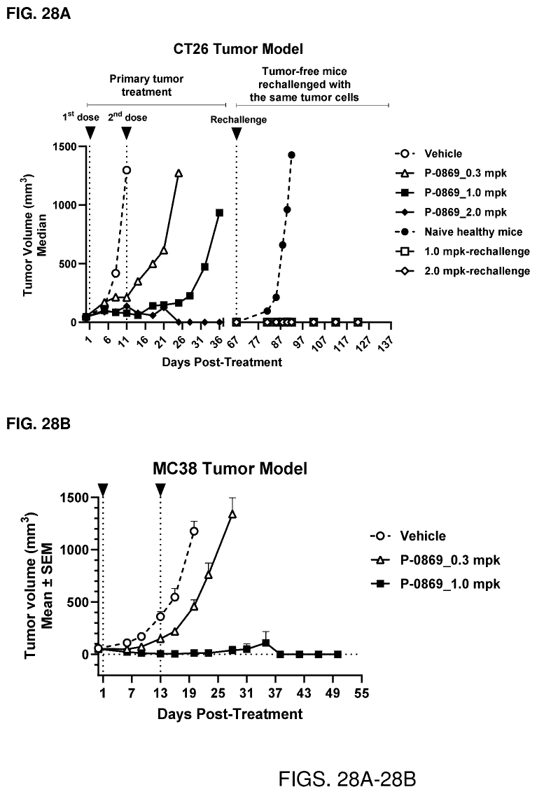

P-0869 at various dosing levels (0.3, 1.0, and 2.0 mg/kg) were administered every 10 days (Q10D) for a total of 2 injections on Days 1 and 11 to CT26 tumor-bearing mice. Each dose is marked with a dotted line and an arrow. Vehicle (sterile PBS) was included as the negative control. Media tumor volumes for each group as a function of time were illustrated in FIG. 28A. Mice treated with Vehicle rapidly developed large subcutaneous tumors. P-0869 demonstrated a potent efficacy in inhibiting tumor growth in a dose-dependent manner. Remarkably, tumors were entirely eradicated in three mice: one from the 1 mg/kg dose group and two from the 2 mg/kg dose group. Given the generally reduced effectiveness seen with the CT26 tumor model when subjected to PD1 therapy, the observed anti-cancer results are notable.

The 3 mice that remained tumor-free were then reintroduced to a CT26 cell implantation on Day 67 post-initial treatment, or 75 days post-primary implantation. As shown in FIG. 28A, none of the rechallenged mice had tumor recurrence, in comparison to the successful engraftment observed in age-matched, naïve mice used as the control. It is evident that the PD1 Ab-IL-15 immunocytokine induced long-term immunity.

P-0869 also effectively suppressed the growth of MC38 tumors in a dose-dependent manner, as illustrated in FIG. 28B. Two doses of P-0869 were administered on Days 1 and 13, with dosing levels of 0.3 and 1 mg/kg. Vehicle (sterile PBS) was included as the negative control, and each group consisted of 8 mice. On Day 19, when compared to the Vehicle-treated group, the tumor growth inhibition (TGI) was 64% for the group treated with 0.3 mg/kg P-0869 and reached a full 100% for the 1 mg/kg group. Notably, in the group receiving 1 mg/kg dosage, 6 of the 8 mice saw total tumor eradication.

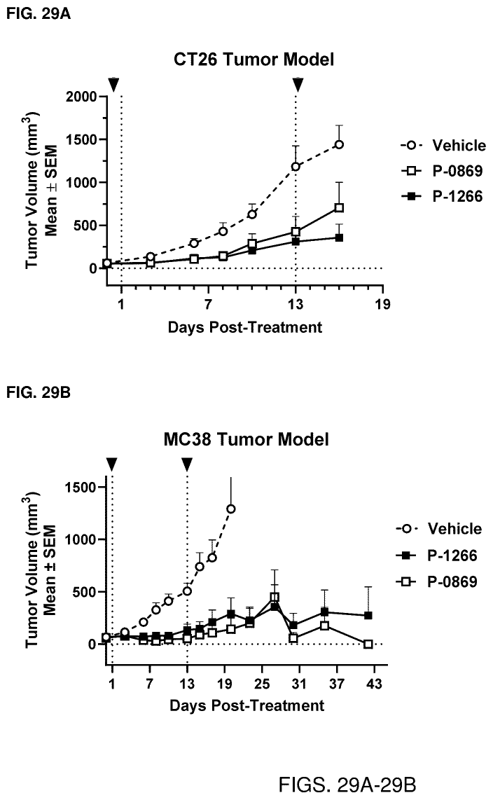

As described in Example 12, dimeric IL-15 in P-0869 triggered far less CD8 T and NK cell expansion than its monomeric equivalent, P-1266, despite a comparable potency in stimulating Ki67 expression on the same lymphocytes. Nonetheless, when considering their anti-tumor effectiveness, both P-1266 and P-0869 yielded similar results in CT26 tumor (FIG. 29A) and MC38 tumor models (FIG. 29B). In the CT26 tumor model, each compound was administered in two Q12D doses of 1 mg/kg. Post-treatment with P-0869, 2 out of 7 mice were left tumor-free, while for the P-1266 group, 1 out of 7 mice achieved total tumor eradication. Similarly, in the MC38 tumor model, each mouse received two doses of 1.5 mg/kg on Days 1 and 13. The P-0869 treatment led to 3 out of 7 mice being tumor-free, while 4 out of 7 mice from the P-1266 group witnessed complete tumor removal. In both cases, P-1266 and P-0869 yielded comparable TGI.

In a comparable manner, P-1295 and P-1296 were similarly assessed in CT26 and MC38 tumor models. Both P-1295 and P-1296 contain the Q108N mutation which hinders their interaction with the γc receptor subunit. Additionally, P-1296 has an 168H mutation impacting the IL-15Rβ interface. Their in vitro activity comparison was shown in FIG. 23. Compared to the wild-type IL-15 counterpart, P-1284, the EC₅₀ values changed from 0.13 nM to 1.94 nM for P-1266, 4.93 nM for P-1295, and 44.3 nM for P-1296, representing potency reductions of by 15, 38, and 340 times. The immune cell expansion triggered by P-1295 and P-1296 was markedly less than that by P-1266 (illustrated in FIGS. 24 and 25). This difference is likely attributed to the varied receptor subunits affected, which subsequently alters the signaling cascade driving cell expansion.

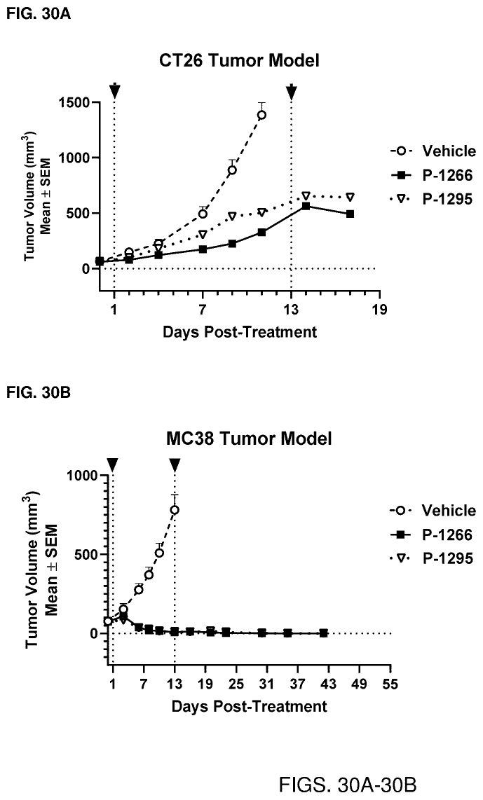

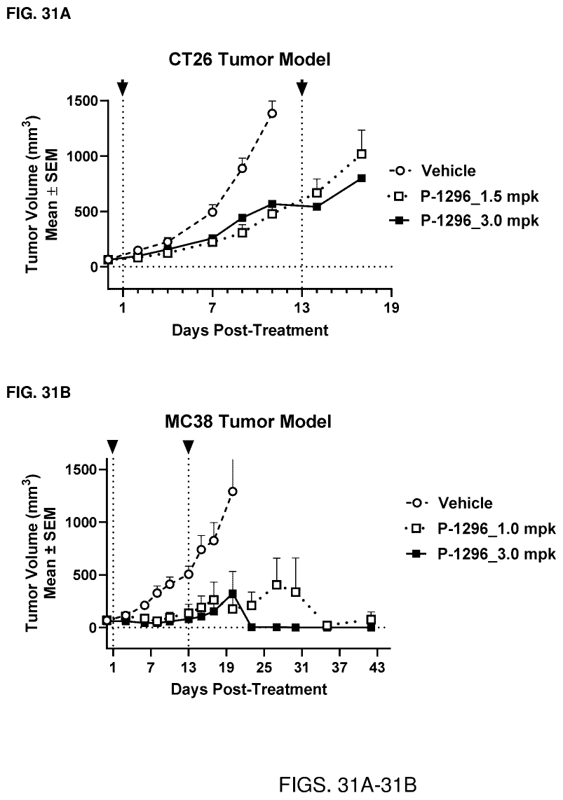

Despite the observed reduction in immune cell expansion, both P-1295 and P-1296 exhibited anti-tumor effectiveness in both MC38 and CT26 models. Specifically, with two doses at 1.5 mg/kg, P-1295's effectiveness closely matched that of P-1266, as depicted in FIGS. 30A and 30B. Remarkably, in the MC38 tumors, both compounds achieved full tumor elimination in all eight mice (FIG. 30B). Furthermore, P-1296, at doses of 1.5 and 3 mg/kg, significantly inhibited tumor growth in CT26 (TGI of 72% and 64% on Day 9, shown in FIG. 31A) and MC38 models (TGI of 97% and 100% on Day 17, with 3 and 5 out of 7 mice becoming tumor-free, as illustrated in FIG. 31B).

In summary, PD1 Ab-IL-15 immunocytokines with IL-15 variants of a range of agonist potencies impacting different receptor subunits all demonstrated anti-tumor capabilities. These results were consistently observed when the compounds were assessed in CT26 and MC38 tumor models. This emphasized the potential versatility and efficacy of PD1-Ab IL-15 immunocytokines in addressing multiple tumor types.

### Example 14

### Tuning IL-15 VitoKines' Intrinsic Basal Activity with IL-15 Variants of Varying Potency Levels

Compared to PD1 Ab-IL15 immunocytokines with attenuated IL-15Rβγ activity, VitoKine platform may provide a more sophisticated strategy to balance antibody and cytokine components, prevent pathway over-activation, and minimize antigen sink as well target-mediated deposition. This enhances the safety profile and bioavailability, potentially allowing human dosing within the effective range of a PD1 antibody. The Vitokine technology is disclosed in WO2019246392 and WO2021119516 by the current inventors. In such a construct, a cytokine domain's activity is concealed until activated locally by tumor-associated antigens. Despite an efficient activity concealment by over 1000-fold with an optimal L2 linker and concealing domain, a VitoKine with a highly potent IL-15 domain might still exhibit significant intrinsic basal activity.

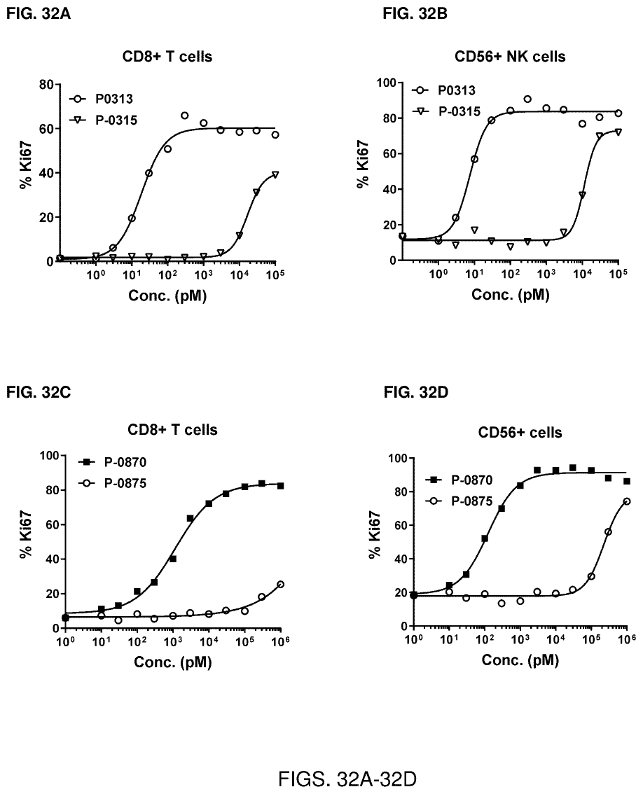

For instance, P-0315, an Fc dimeric IL-15 VitoKine, harboring a fully active IL-15 S58D variant, has an intrinsic basal activity capable of stimulating CD8 T cells with an EC₅₀ of 30 nM and NK cells with an EC₅₀ of 11 nM in human PBMCs. This is in spite of an over three orders of magnitude decrease in activity compared to its non-VitoKine counterpart P-0313 (FIGS. 32A and 32B). When administer at in vivo dosages higher than 1 mg/kg, P-0315's inherent basal activity could stimulate peripheral receptors persistently and lead to prolonged in vivo pharmacodynamic effect, posing risks of systematic toxicity (data not shown).

Incorporating IL-15 variants of lower potency helps tune the intrinsic basal activity of IL-15 VitoKines, which proportionally correlates with the L-15 moiety's activity. For example, P-0875, a PD1 Ab-IL-15 VitoKine that includes the IL-15 V63A/168H variant as the D2 domain, displays a significantly reduced intrinsic basal activity due to the weakening of the IL-15 domain. In line with the 1000- to 2000-fold concealment efficiency characteristic of this IL-15 VitoKine platform, P-0875′ is approximately 1000-2000-fold less potent than P-0870, its non-VitoKine counterpart. The estimated EC₅₀ values for P-0875 are ˜2 μM and 225 nM in stimulating proliferation of human CD8 T cells and NK cells respectively as illustrated in FIGS. 32C and 32D. Given their higher sensitivity to IL-15 compared to CD8 T cells, NK cells were included in the assessment to evaluate the ex vivo activity of IL-15 VitoKine constructs, especially those with lower intrinsic basal activity.

In a preliminary pharmacodynamic study using cynomolgus monkeys, P-0875 was administered at a dosage exceeding the typical effective range of a PD1 antibody in humans. Despite this elevated dose, there was only a minimal increase in cytotoxic lymphocytes in peripheral blood and no adverse events were observed. This notably enhanced safety profile can be attributed to the adoption of an attenuated active domain, thereby leading to reduced intrinsic basal activity of the VitoKine.

Importantly, the tunable IL-15 VitoKine intrinsic basal activity allows for a fine balance between the VitoKine's activity inertness prior to cleavage and its potency upon activation. This facilitates achieving the desired anti-tumor efficacy while minimizing the risk of the unintended systemic toxicity. Any IL-15 variants, each with differing potency levels as disclosed in this invention, including those defined by SEQ ID NOS: 116-163, can be used as the active moiety domain to construct PD1 Ab-IL-15 VitoKines.

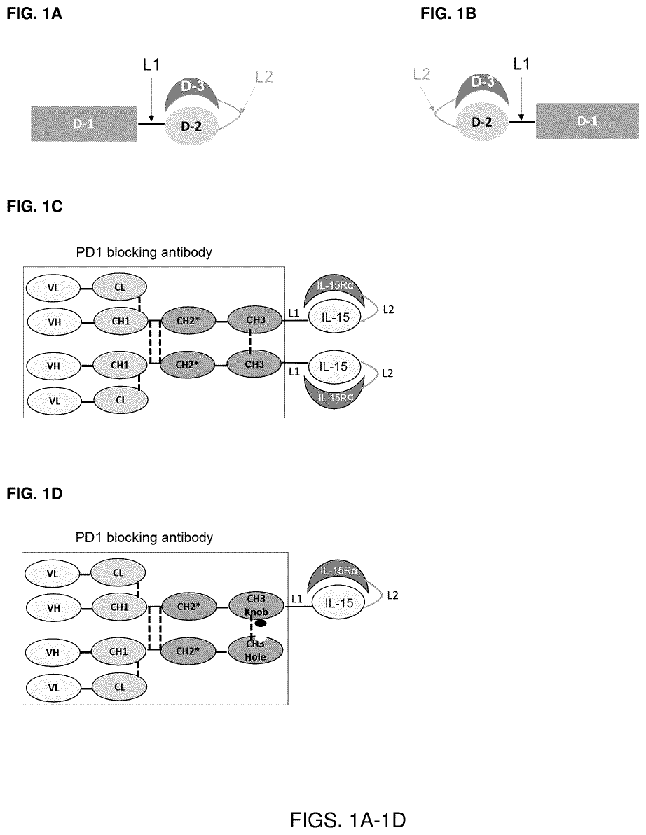

Furthermore, the valency of the IL-15 domain in PD1 Ab-IL-15 VitoKines can be adjusted to further tune their intrinsic basal activity as well as the potency upon proteolytic activation. Both dimeric and monomeric PD1 Ab-IL-15 VitoKines, with their structures respectively illustrated in FIG. 1C and FIG. 1D, were constructed.

### Example 15

### Optimizing IL-15 VitoKine's Developability and Activity Through Adopting Varied L2 Linkers

IL-15 VitoKine constructs comprising a 10-amino acid MMP-2/9 cleavable L2 linker ‘GGPLGMLSQS’ (SEQ ID NO: 85) tend to have inferior protein developability profiles compared to IL-15 fusion proteins with a non-covalently associated IL-15RαSushi+ domain. Among them, P-0874 and P-0869 represent such a pair. The structure of P-0874, a PD1 Ab-IL-15 VitoKine, is shown in FIG. 1C, and its molecular details can be found in Table 23B. P-0869, with its structure illustrated in FIG. 3A, is the non-VitoKine counterpart of P-0874, with IL-15RαSushi+ domain (SEG ID NO: 165) introduced non-covalently during co-expression.

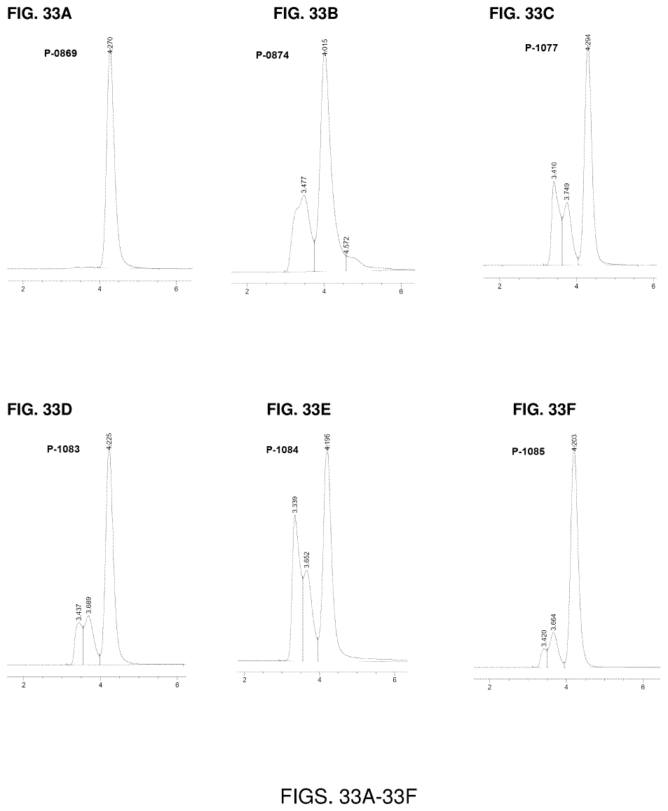

From the size-exclusion chromatography (SEC) analysis of P-0869 and P-0874, as illustrated in FIGS. 33A and 33B, P-0874 clearly displays poorer purity. After one protein A purification step, P-0869 contains 37.6% of impurities, primarily attributed to aggregates and a minor fragment content, which is in sharp contrast to the 100% purity for P-0874. Furthermore, P-0869 was produced at a significantly reduced level compared to P-0874, in addition to its increased tendency to aggregate.

The suboptimal expression profiles of IL-15 VitoKines could be attributed to the spatial constraints introduced by the L2 linker, which may lead to distorted interactions between IL-15 and IL-15RαSushi domains. It is postulated that by adjusting the L2 linker's length and/or varying its sequence/flexibility, the developability and biophysical attributes of the resulting IL-15 VitoKines might be enhanced. Given that the L2 linker length and composition play a crucial role in the efficiency of D3 concealment, there is a need to strike a balance to ensure that the VitoKine's activity inertness remains mostly unaltered. Following the above considerations, the L2 linker in P-0874 was replaced with linkers of varying lengths and compositions. The resulting PD1 Ab-IL-15 VitoKine constructs are listed in Table 21. For instance, the L2 linker in P-0874 has 10 amino acids, whereas it has 15 amino acids in P-1077, P-1083, P-1084, and 20 amino acids in P-1085.

TABLE 21
Sequences and protease targets for L2
Linkers in PD1 Ab-IL-15 VitoKines

| IL-15 | | L2 Linker | Protease |
|---|---|---|---|
| VitoKine | L2 Linker Seq | SEQ ID NO: | Targets |
| P-0874 | GGPLGMLSQS | 85 | MMP-2/9 |
| P-1077 | GGSGPLGMLSQGGGS | 84 | MMP-2/9 |
| P-1083 | GGGGSPMAKKGGGGS | 91 | Matriptase |
| P-1084 | GGPLGMLSQPMAKKS | 92 | MMP-2/9 & |
| | | | Matriptase |
| P-1085 | GGSGPLGMLSQPMAK | 93 | MMP-2/9 & |
| | KGGGS | | Matriptase |

The protein A purified material's expression level (in mg/L) and purity, as determined by SEC chromatography for the exemplary molecules, are summarized in Table 22. Furthermore, their SEC chromatograms are depicted in FIG. 33. Strikingly, the incorporation of the 20-amino acid dual protease-cleavable L2 linker (SEQ ID NO: 93) in P-1085 considerably enhanced its developability profiles. There was a markedly lowered aggregation propensity, with purity improved from 62% for P-0874 to over 80% for P-1085, as indicated by SEC (FIG. 33 and Table 22). Additionally, the productivity saw a considerable increase, changing from roughly 20 mg/L to 91 mg/L. Such improvement in developability was only seen when the linker was elongated from 10 to 20 amino acids, not when the linker length was increased to 15 amino acids.

Even more strikingly, P-1084, which contains a 15-amino acid L2 linker (SEQ ID NO: 92) of the identical core sequence (GPLGMLSQPMAKK; SEQ ID NO: 76) as in P-1085, not only displayed poorer expression compared to RO1085 (as listed in Table 22). it also showed a minor (<10%) premature cleavage during the cell culturing process (data not shown). This observation suggests that the peptide spacers flanking the cleavable linkers could introduce extra, undesirable cleavable site(s) in a context-dependent manner. Finally, the composition of the L2 linker, in addition to its length, significantly influenced the developability profile of IL-15 VitoKine. This is evidenced by the data for P-1347. Even though its only difference from P-1085 is the L2 linker composition, which is a 20-amino acid non-cleavable flexible linker, GGGGSGGGGSGGGGSGGGGS (SEQ ID NO: 102), P-1347 exhibited poor purity at 50% and a diminished expression level of 19.2 mg/L.

TABLE 22
Expression, purity, and ex vivo activity of exemplary
PD1 Ab-IL-15 VitoKines with different L2 linkers

| IL-15 | Titer | Monomer % | % Ki67 expression EC₅₀ (nM) | |
|---|---|---|---|---|
| VitoKine | (mg/L) | by SEC | CD8 T cells | NK cells |
| P-0874 | 19.7 | 62.4 | >1000 nM | ~300 |
| P-1077 | 33.7 | 54.8 | ~1000 nM | 282 |
| P-1083 | 25.8 | 69.5 | ~1000 nM | 93.1 |
| P-1084 | 39.3 | 57 | 63.9 | 3.95 |
| P-1085 | 91 | 80.5 | 372 | 33.1 |

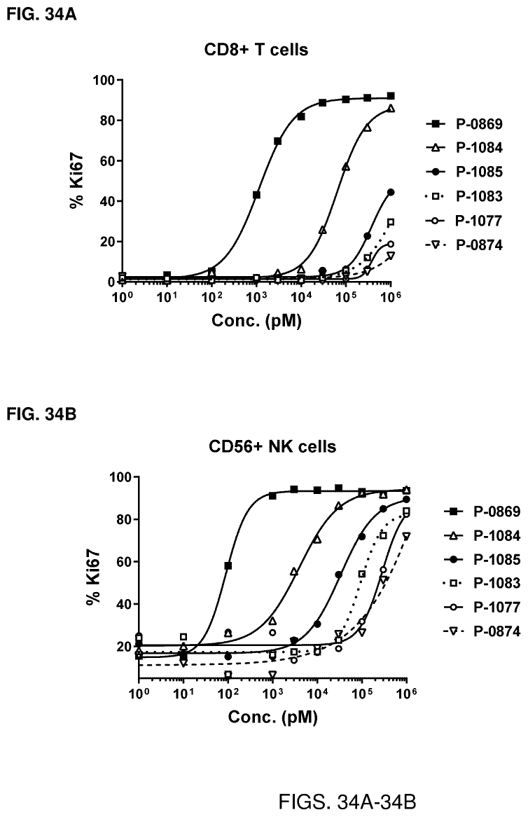

The biological function of the VitoKine constructs was assessed by measuring the increases in Ki67 expression in CD8 T cells and NK cells within human PBMCs. The data can be seen in FIG. 34 with EC₅₀ values summarized in Table 22. The EC₅₀ values for P-0869, their non-VitoKine fusion counterpart, were 1.14 nM for CD8 T cells and 0.089 nM for NK cells. The pre-mature cleavage of the L2 linker in P-1084, which was neither expected nor desirable, resulted in unwanted elevation in its basal activity. Compared to P-1083, which features an L2 linker of identical length, P-1084 displayed a basal activity that was 25 times higher, with EC₅₀ values of 3.95 nM for P-1084 and 93.1 nM for P-1083 in stimulating NK cell proliferation.

As anticipated, the extension of the L2 linker, from 10 amino acids in P-0874 to 15 in P-1083, and further to 20 in P-1085, led to incremental enhancement in activity for both CD8 T and NK cells (FIG. 34). In direct comparison with P-0869, P-1085 demonstrated an overall concealing efficiency around 350-fold. This efficiency captures a potency reduction of 325-fold for CD8 T cells and 371-fold for NK cells and is a modest reduction from the concealing efficiency of 1000-2000-fold when the 10-amino acid cleavable linker (SEQ ID NO: 85) was used as the L2 linker.

The 20-amino acid dual protease-cleavable linker (SEQ ID NO: 93) in P-1085 was subsequently integrated into two additional PD1 Ab-IL-15 VitoKines: P-1265 and P-1263. P-1265 is the monomeric counterpart of P-1085 comprising IL-15 V63A/168H variant. P-1263 and P-1265 differ solely in the IL-15 domain, with P-1263 containing the Q108N mutation. Both exhibited markedly better developability profiles compared to the IL-15 VitoKine with a 10-amino acid L2 linker, exemplified by P-0874. Notably, P-1263 exhibited a purity of 90% purity, and P-1265 achieved 85% purity, both had decent expression levels exceeding 50 mg/L.

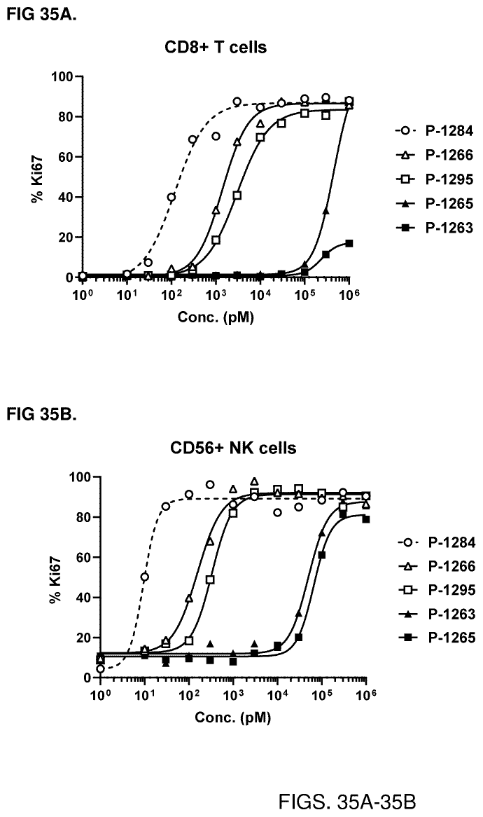

The activity of P-1265 and P-1263 was assessed in human PBMCs for their capability in stimulating Ki67 expression in CD8 T and NK cells. As illustrated in FIG. 35, both compounds exhibited a roughly 300-fold decrease in activity when compared to their respective non-VitoKine counterparts, namely P-1266 for P-1265 and P-1295 for P-1263. This diminished activity is characteristic of the concealing efficiency associated with the 20-amino acid L2 linker. Additionally, the basal activity of the VitoKine proportionally correlates with the activity of its L-15 moiety.

In summary, the incorporation of the 20-amino acid MMP and matriptase dual cleavable linker ‘GGSGPLGMLSQPMAKKGGGS’ (SEQ ID NO: 93) notably improved the developability profiles of IL-15 VitoKines. However, the incorporation of a longer linker slightly reduce the concealing efficiency, leading to VitoKines that had relatively higher inherent basal activity. If a lower basal activity is preferred, tuning the level of inertness could be achieved by incorporating a less potent IL-15 variant. Furthermore, altering the valency of the cytokine domain provides another approach to modulate VitoKine's inertness.

### Example 16

### Constructing PD1 Ab-IL-15 VitoKines with Optimized Components

PD1 Ab-IL-15 VitoKines comprising a PD1 blocking antibody as the targeting domain (D1), an IL-15 or IL-15 variant as the active moiety domain (D2), and an IL-15RαSushi+ domain (SEQ ID NO: 165) as the concealing moiety domain (D3) are illustrated in FIG. 1C (dimeric IL-15) and FIG. 1D (monomeric IL-15).

It is desirable to construct PD1 Ab-IL-15 VitoKines with PD1 antibodies of superior PD1 binding and blocking activities to reverse T-cell anergy or exhaustion and synergize with IL-15 anticancer immune response. The PD1 antibodies used to construct PD1 Ab-IL-15 VitoKines were selected from the optimized human PD1 blocking antibodies comprising light chain sequences set forth in SEQ ID NO: 44 and heavy chain sequences set forth in SEQ ID NOS: 45-49. These optimized PD1 blocking antibodies have a high affinity for human PD1 protein and demonstrate equal or comparable potency as pembrolizumab in blocking PD1. They also possess a higher sequence similarity score to their closest human germline sequence, resulting in an improved degree of humanness compared to pembrolizumab. Furthermore, they are predicted to have lower hydrophobicity, which in turn is likely to lower their aggregation propensity than pembrolizumab. PD1-targeted IL-15 VitoKines constructed using these optimized PD1 blocking antibodies are also projected to have enhanced developability profiles.

Both the L1 linker connecting D1 and D2 and the L2 linker connecting D2 and D3 can be cleavable and non-cleavable, but the L2 linker is preferably cleavable. Cleavage of the L2 linker leads to Active Form 2 (depicted in FIG. 2), which is a fully functional IL-15 domain along with a non-covalently complexed IL-15RαSushi fused to the PD1 Ab. This form can activate IL-2R signaling in PD1-expressing T cells near the disease site, enhancing both pathways and synergizing the anticancer immune response, while reducing systemic toxicity. On the other hand, if the L1 linker is cleavable, its cleavage results in Active Form 1, which has shorter half-life, exhibits reduced potency, and lacks TIL-targeting capabilities.

By adjusting the length and composition of the L2 linker, the inherent basal activity of VitoKines can be fine-tuned. For instance, a 10-aa MMP-2/9 cleavable L2 linker (SEQ ID NO: 85) typically results in a concealing efficiency of 1000- to 2000-fold. In contrast, a 20-aa MMP and matriptase dual cleavable L2 linker (SEQ ID NO: 93) leads to an approximately 350-fold concealment efficiency. In addition to intrinsic basal activity adjustment, the dual cleavable L2 linker notably enhanced the developability profiles IL-15 VitoKines. The sequence of the cleavable linkers can be further refined to better suit various tumors. Furthermore, by incorporating IL-15 variants with varying potency, the intrinsic basal activity of IL-15 VitoKines can be modulated. Table 23A lists the exemplary PD1 Ab-IL-15 immunocytokine constructs.

TABLE 23A
Exemplary human PD1 Ab-IL-15 VitoKine constructs

| IL-15 | VitoKine | IL-15 | IL-15 | Linker 2 (L2) |
|---|---|---|---|---|
| VitoKine | SEQ ID NOs: | mutations | Valency | SEQ ID NO: |
| P-0875 | 181 + 50 | V63A/I68H | Dimeric | 85 |
| P-1349 | 182 + 44 | V63A/I68H | Dimeric | 93 |
| P-1339 | 183 + 174 + 44 | V63A/I68H | Monomeric | 93 |
| P-1340 | 184 + 174 + 44 | Q108N | Monomeric | 93 |
| P-1350 | 185 + 44 | Q108N | Dimeric | 93 |

All genes were codon optimized for expression in mammalian cells, which were synthesized and subcloned into the recipient mammalian expression vector through the service of GenScript. The VitoKine constructs were produced by co-transfecting Expi293 cells (ThermoFisher) with the mammalian expression vectors following manufacturer's instructions. Protein purification and characterization were carried out in accordance with the procedures outlined in Example 2.

Since the human PD1 blocking antibodies of the present invention did not bind to mouse PD1, surrogate mouse PD1 Ab-IL-15 immunocytokines were constructed using a mouse PD1 antibody in a similar manner. They were made for in vivo studies to assess the pharmacodynamic effects on stimulating lymphocytes proliferation and expansion in mice, as well as to evaluate their efficacy in inhibiting tumor growth in syngeneic mouse tumor models. Table 23B lists the exemplary surrogate mouse PD1 Ab-IL-15 VitoKine constructs. The L1 linker in all VitoKines in Table 23B is a non-cleavable ‘GGGGSGGGGSGGGGS’ linker (SEQ ID NO: 115). In the case of P-0878, P-1347, and P-1264, they are the non-cleavable equivalents of P-0874, P-1265, and P-1264, respectively. The contained length matched, noncleavable L2 linkers that corresponded to their respective VitoKines.

TABLE 23B
Exemplary Surrogate mouse PD1 Ab-IL-15 VitoKine constructs

| IL-15 | Component PD1 | IL-15 | IL-15 | IL-15 | Linker 2 (L2) |
|---|---|---|---|---|---|
| VitoKine | Ab SEQ ID NOS: | mutations | SEQ ID NO: | Valency | SEQ ID NO: |
| P-0874 | 53 + 52 | V63A/I68H | 132 | Dimeric | 85 |
| P-0878 | 53 + 52 | V63A/I68H | 132 | Dimeric | 114 |
| P-1077 | 53 + 52 | V63A/I68H | 132 | Dimeric | 84 |
| P-1083 | 53 + 52 | V63A/I68H | 132 | Dimeric | 91 |
| P-1084 | 53 + 52 | V63A/I68H | 132 | Dimeric | 92 |
| P-1085 | 53 + 52 | V63A/I68H | 132 | Dimeric | 93 |
| P-1265 | 169 + 170 + 52 | V63A/I68H | 132 | Monomeric | 93 |
| P-1347 | 169 + 170 + 52 | V63A/I68H | 132 | Monomeric | 102 |
| P-1263 | 169 + 170 + 52 | Q108N | 142 | Monomeric | 93 |
| P-1264 | 169 + 170 + 52 | Q108N | 142 | Monomeric | 102 |
| P-1346 | 53 + 52 | Q108N | 142 | Dimeric | 93 |
| P-1248 | 53 + 52 | Q108N | 142 | Dimeric | 102 |

### Example 17

### In Vitro Activity and Proteolytic Activation of PD1 Ab-IL-15 VitoKines

It is essential that the PD1 antibody retains its binding and functional activities when incorporated into PD1 Ab-IL-15 VitoKines. PD1 antibodies of superior target-binding and PD1 blocking function can enhance the specificity and selectivity of TIL-targeting, and further synergize with IL-15 anticancer immune response by efficiently reversing T cell anergy and exhaustion.

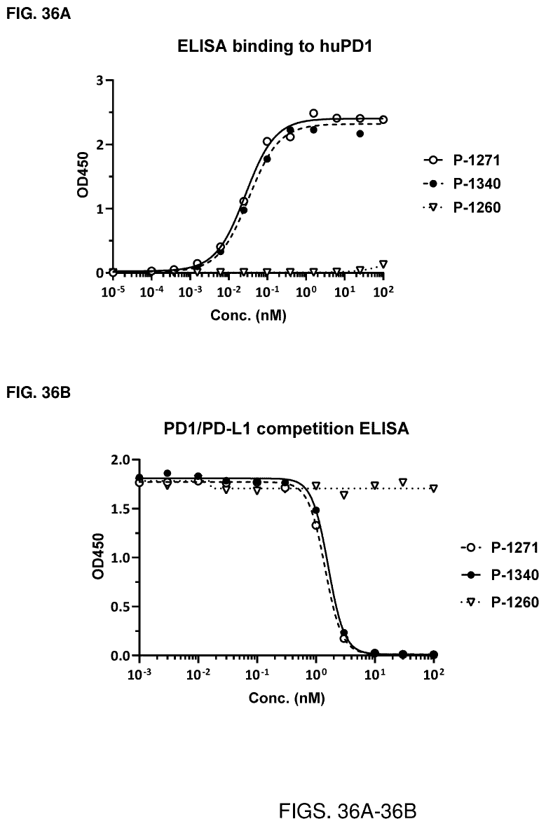

Two ELISA assays were conducted to evaluate the PD1 binding and inhibition capabilities of the exemplary IL-15 VitoKine P-1340 against its component PD1 antibody, P-1271. Additionally, a non-targeting germline antibody, P-1260 (SEQ ID NOS: 171, 172, and 173) was included as the negative control. The binding ELISA method is outlined in Example 3, while the competition ELISA is described in Example 11. FIG. 36A shows that both P-1271 and P-1340 exhibit undistinguishable PD1 binding capabilities, demonstrated by their EC₅₀ values of 26.8 pM and 32.8 pM, respectively. Similarly, FIG. 36B reveals their consistent efficiency in blocking PD-L1 from binding to PD1 with equal potency with IC50 values of 1.42 nM for P-1271 and 1.51 nM for P-1340. These findings underscore that the PD1 antibody fully retained its binding and blocking activity when incorporated into VitoKine constructs.

In addition to confirming that the IL-15 moiety activity can be efficiently concealed by IL-15RαSushi+ domain and remain inert regardless of the PD1 antibody sequence compositions, it is also crucial to verify that PD1 Ab-IL-15 VitoKines can be efficiently cleaved and activated to fully restore the activity of the IL-15 moiety. The presence of a bulky antibody may spatially hinder the protease accessibility and prevent efficient cleavage.

An exemplary PD1 Ab-IL-15 VitoKine, P-0875, was assessed for protease cleavage and subsequent activation of the IL-15 domain. P-0875 contains a single MMP-2/9 cleavable linker ‘GGPLGMLSQS’ (SEQ ID NO: 85) connecting IL-15 V63A/168H variant and IL-15RαSushi+ domains. Briefly, 3.3 μg of latent MMP-2 (BioLegend) was first activated by APMA (Millipore Sigma) according to the manufacturer's instruction, which was then buffer exchanged and added to 120 μg P-0875 in 0.4 ml of the manufacture recommended assay buffer (100 mM Tris, 20 mM CaCl₂), 300 mM NaCl, 0.1% (w/v) Brij 35, pH 7.5). After incubation at 37° C. for 2 hours, the digested sample was then purified with protein A resin using bind-elute mode, and the eluted sample was analyzed in a reduced SDS-PAGE gel and its biological function was assessed in an ex vivo functional assay.

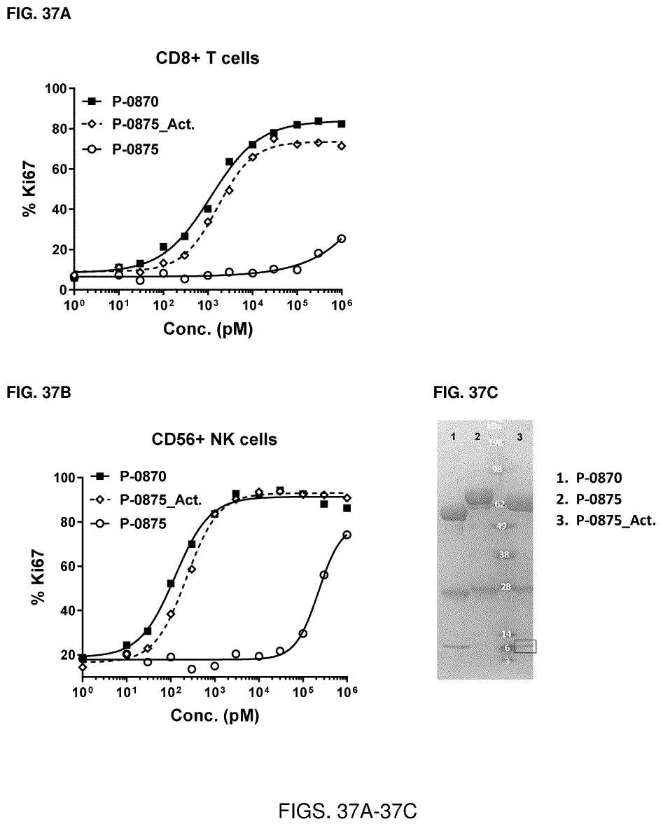

As depicted in FIG. 37C, the appearance of the IL-15RαSushi+ domain as a sharp band at ˜9 KDa on the gel (boxed) confirmed the efficient cleavage at the MMP-2/9 substrate peptide linker. The presence of the IL-15RαSushi+ domain after protein A elution also suggested that the IL-15RαSushi+ domain released from the covalent linkage remain non-covalently associated with IL-15. Such association was strong enough to withstand low-pH conditions during Protein A elution. FIGS. 37A and 37B further demonstrated the activity inertness of the VitoKine and an approximately 2000-fold potency restoration in both NK cells and CD8+ T cells after in vitro proteolytic activation, which brought the activity back to the level matching that of the non-VitoKine PD1 Ab-IL-15 fusion counterpart P-0870.

VitoKines P-1340 and P-1349 underwent similar assessment. They also demonstrated activity inertness as the intact molecule with approximately 350-fold concealing efficiency when compared to their respective non-VitoKine counterparts, P-1380 and P-1369, and full activity restoration upon in vitro protease cleavage. They were also confirmed to be cleavable by both MMP-2/9 (BioLegend) and matriptase (R&D systems).

### Example 18

### PD1-Ab-IL-15 VitoKines Minimized Systemic Pharmacodynamic Effects in Non-Tumor Bearing Mice

The VitoKine platform is designed to reduce systemic toxicity and widen the therapeutic window. By maintaining the active cytokine in an inert state, it avoids interactions with receptors on healthy cells, curtailing unintended cytokine pathway activations and minimizing adverse effects. To validate this, healthy C57BL/6 mice were administered with P-1265 to compare its pharmacodynamic effects on peripheral immune cells against those elicited by P-1266, its non-VitoKine counterpart. This experiment was conducted following similar procedures detailed in Example 12.

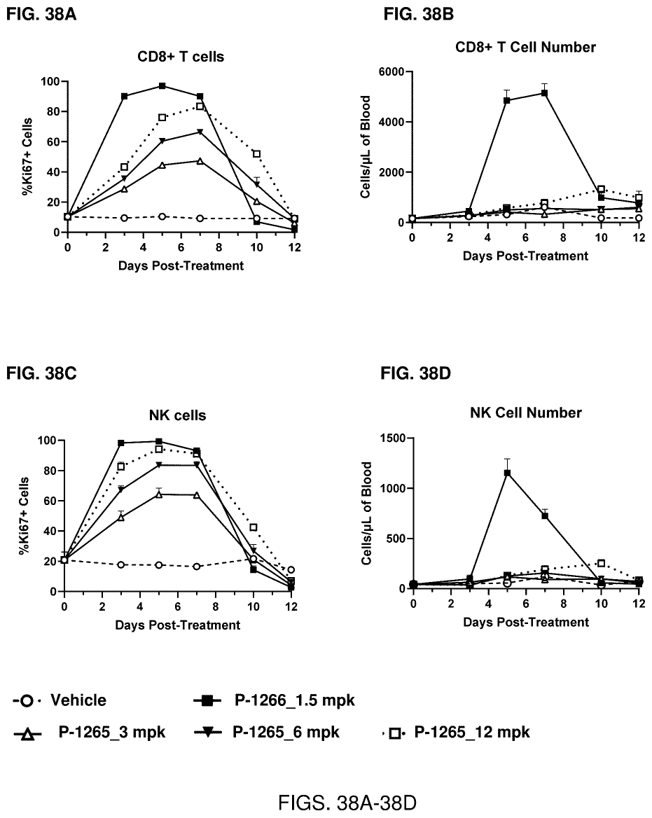

As illustrated in FIGS. 38A and 38C, at a dose of 1.5 mg/kg, P-1266, the non-VitoKine counterpart of P-1265, induced maximum Ki67 expression (100%) in both CD8 T and NK cells. The expression remained consistently high between Day 3 and Day 7 before rapidly declining to baseline by Day 10. On the other hand, P-1265, the PD1 Ab-IL-15 VitoKine with the monomeric IL-15 V63A/168H variant, showed dose-dependent elevations in Ki67 expression for both CD8 T and NK cells. Specifically, for CD8 T cells, the expression peaked at 47%, 66%, and 84% on Day 7 for substantially higher dosages of 3, 6, and 12 mg/kg, respectively (FIG. 38A). For NK cells, peak levels of 64%, 84%, and 94% were observed on Day 5 for doses of 3, 6, and 12 mg/kg, respectively (FIG. 38C).

However, for cell expansion, P-1265 demonstrated a drastically different profile from P-1266. As illustrated in FIGS. 38B and 38D and summarized in Table 24, P-1266 dosed at 1.5 mg/kg promoted the expansion of CD8 T cells by 33-fold from baseline which peaked on Day 7 and NK cells by 27-fold on Day 5. The increases in CD8 T and NK cell numbers are markedly lower even at the 12 mg/kg dose, an 8-fold higher than the dose of P-1266. For the lower doses of 3 and 6 mg/kg, the expanded cells remained relatively constant from Day 5 through Day 12, the end of the study.

TABLE 24
Peripheral immune cell expansion in response to various treatments

| | | CD8 T cells | | | NK cells | | |
|---|---|---|---|---|---|---|---|
| | Dose | | Days Post- | Fold | | Days Post- | Fold |
| Compound | (mg/kg) | Cells/μL | Treatment | Change | Cells/μL | Treatment | Change |
| P-1266 | 1.5 | 5150 | 7 | 33 | 1153 | 5 | 27 |
| P-1265 | 3 | 552 | 12 | 3.5 | 117 | 5 | 2.7 |
| P-1265 | 6 | 606 | 12 | 3.8 | 156 | 7 | 3.6 |
| P-1265 | 12 | 1315 | 10 | 8.3 | 251 | 10 | 5.8 |

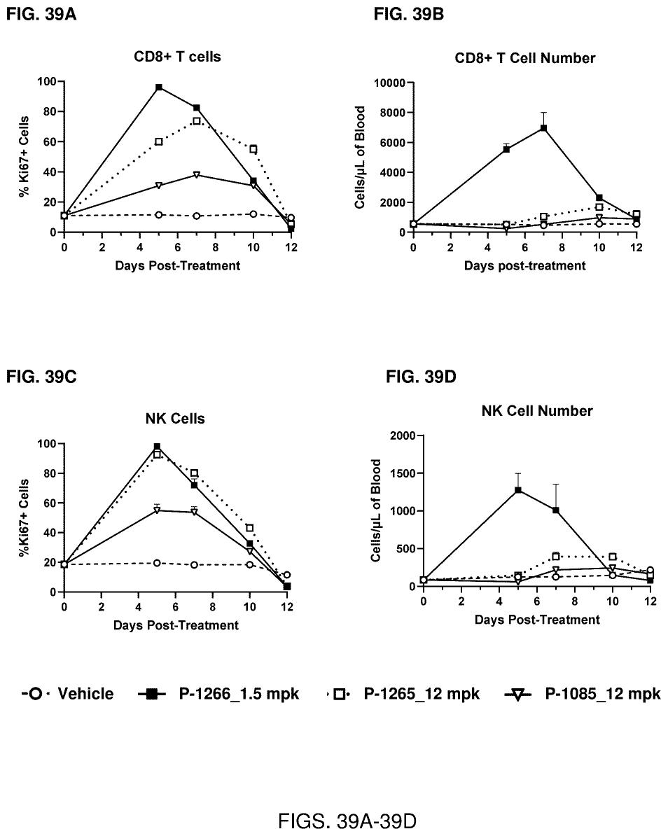

In a parallel in vivo study using naïve C57B/L6 mice, the pharmacodynamic effects of the PD1 Ab-IL-15 VitoKine, P-1265, were assessed alongside its dimeric VitoKine equivalent, P-1085, and its non-VitoKine counterpart, P-1266. A single dosage of 12 mg/kg was administered for P-1265 and P-1085, whereas P-1266 was dosed at 1.5 mg/kg. Blood samples were collected on Days 0, 5, 7, 10, and 10 for lymphocyte analysis using flow cytometry. FIG. 39 reveals that both VitoKine format, monomeric and dimeric, induced a more notable increase in Ki67 expression on peripheral lymphocytes (FIGS. 39A and 39C) compared to the expansion of these cells (FIGS. 39B and 39D). However, this increase is still substantially lower than that of their active non-VitoKine counterpart. Recalling observations from Example 12 and FIG. 27, the dimeric IL-15, previously in immunocytokine format and now in VitoKine form, had less pronounced effects than its monomeric counterpart, opposing in vitro findings. Specifically, at 12 mg/kg, peak Ki67 expression was 74% for P-1265 and 38% for P-1085 (FIG. 39A). Meanwhile, CD8 cell counts went from a baseline level of 551 cells/μL blood to 1315 cells/μL peak for P-1265 and to 962 cells/μL peak for P-1085 (FIG. 39B). A similar pattern was observed for NK cells (FIGS. 39 C and 39D).

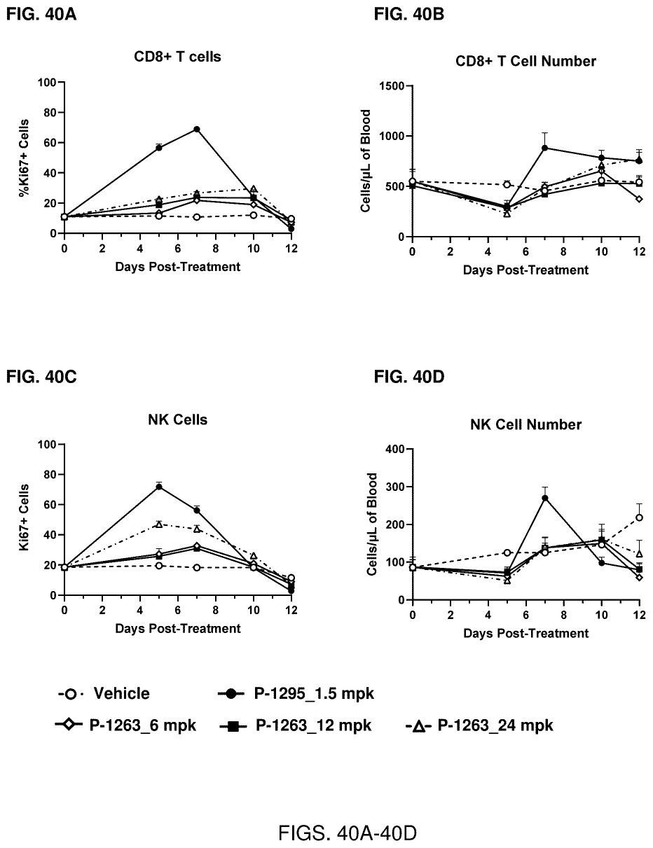

Similarly, the effects of PD1 Ab-IL-15 VitoKine, P-1263, at vary dosing levels (6, 12, and 24 mpk) was compared to its non-VitoKine counterpart, P-1295, dosed at 1.5 mpk, in C57B/L6 mice. Both compounds contain the monomeric IL-15 variant with the Q108N mutation that interferes γc interaction. Following a single intraperitoneal injection, blood samples were collected on Days 0, 5, 7, 10, and 10 for lymphocyte analysis using flow cytometry. As illustrated in FIG. 40, even when dosed at 24 mg/kg, 16 times the dose of P-1295, the only evident pharmacodynamic change from P-1263 was a modest increase in Ki67 expression in NK cells (FIG. 40C). Ki67 expression in CD8 T cells showed only a minor rise (FIG. 40A). Given that the IL-15 variant with γc-disrupting mutations typically led to much less cell expansions than the IL-12 variant with IL-12Rβ mutations (as seen in Example 12 and FIG. 24), it is anticipated that the VitoKine with the γc-disrupting mutations in its IL-15 domain exhibited even less cell expansion. These expected findings are demonstrated in FIGS. 40C and 40D. Notably, even dosed as high as 24 mg/kg, no body weight loss or other signs of stress was observed with P-1263 treatment.

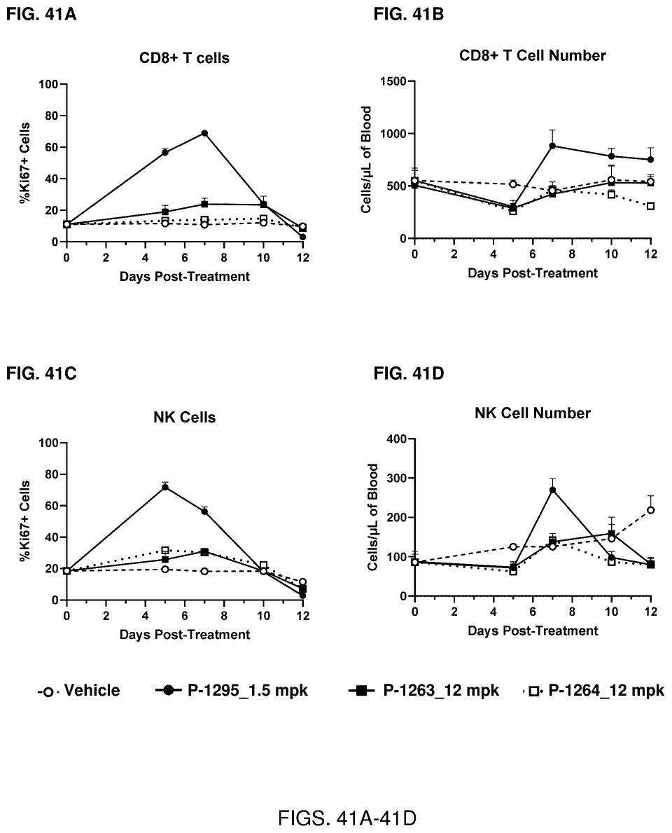

Finally, in a parallel experiment, the pharmacodynamic effects of P-1263 was assessed against its non-cleavable VitoKine equivalent, P-1264, in C57B/L6 mice. The only difference between P-1264 and P-1263 lies in the L2 linker (refers to table 23B for details). While P-1264 displayed identical in vitro activity as P-1263, the IL-15 domain in P-1264 remains concealed and inactive as the D3 domain cannot be cleaved leading to activation. Both P-1263 and P-1264 were administered at 12 mg/kg, while the active non-VitoKine counterpart, P-1295, was dosed at 1.5 mg/kg. Blood samples were analyzed on Days 0, 5, 7, 10, and 10 to phenotype lymphocytes. As illustrated in FIG. 41, no discernible differences in cell proliferation or expansion were observed between the VitoKine and its noncleavable equivalent. suggesting that the VitoKine remains intact in the peripheral blood circulation.

In summary, compared to their active IL-15 non-VitoKine counterparts, IL-15 VitoKines exhibited a marked reduction in systemic proliferation and, particularly, expansion of specific lymphocyte groups, including CD8+ T and NK cells. This highlights the effectiveness of the VitoKine format in concealing IL-15 activity, thereby preventing unwanted activation of the IL-15 pathway and mitigating the risk of undesirable “on-target” effects in “off tissue”. Furthermore, the decrease in systematic pharmacodynamics is more pronounced in VitoKine incorporating IL-15 variants with mutation(s) that disrupt γc interaction and when the IL-15 domain is in a dimeric format. These findings provide added avenue to fine-tune VitoKine's intrinsic basal activity and balance it with its post-activation potency.

### Example 19

### Anti-Tumor Efficacy of PD1 Ab-IL-15 VitoKines in Syngeneic Mouse Tumor Models

The critical role of the VitoKine activation in its anti-tumor efficacy was studied by comparing P-874 and its non-cleavable VitoKine equivalent, P-0878, using the CT26 murine colon carcinoma model. The only difference between P-874 and P-0878 lies in the L2 linker connecting IL-15 (D2) and IL-15RαSushi+ (D3) domains. P-0878 contains a length-matched, noncleavable L2 linker (refers to table 23B for details). While P-0878 displayed identical in vitro activity as P-08874, the IL-15 domain in P-0878 remains concealed and inactive as the D3 domain cannot be cleaved leading to activation.

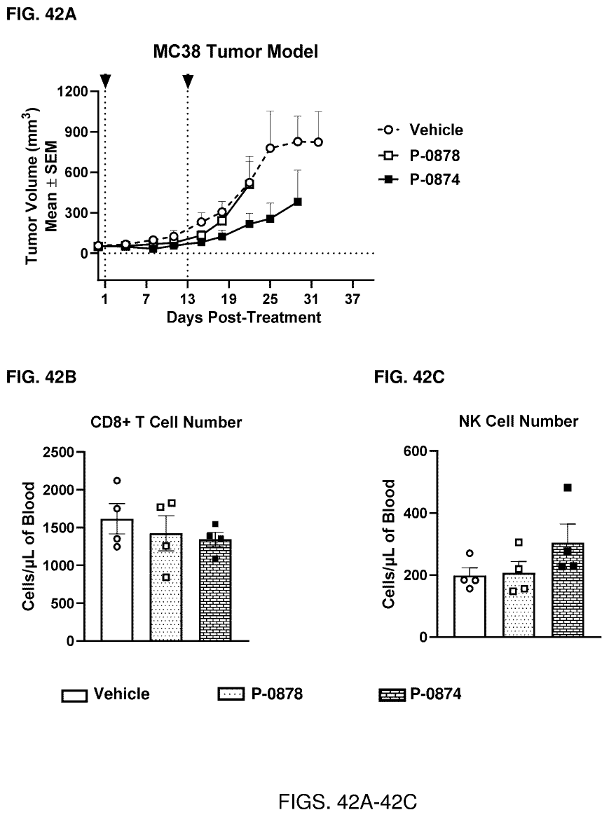

CT26 model was established in the same way detailed in Example 13. P-0874 and P-0878 were administered at a dosage of 10 mg/kg twice following a Q12D dosing schedule, and a Vehicle (sterile PBS) group was included for comparison. As depicted in FIG. 42A, tumors eventually developed in all mice since CT26 is considered as a “cold” tumor” and is less responsive than MC38 model to PD1 therapy. Treatment with P-0878, which has the non-activable inert IL-15 domain, yielded no observable anti-tumor effect. In a sharp contrast, administering P-0784 at the same dosage demonstrated a markedly improved efficacy, showing a 67% TGI on Day 25. This finding suggests that the enhanced anti-tumor effectiveness of VitoKine molecules hinges on the enzymatic cleavage of the linker to release the concealing moiety, thereby activating the IL-15 domain around the tumor.

Notably, there was minimal peripheral immune cell expansion and marginal difference between P-0874 and P-0878 5 days post-the first injection in these tumor-bearing mice (FIGS. 42B and 42C). It is also worth noting that both VitoKines, when given at 10 mg/kg, were well-tolerated with no evidence of weight loss in mice.

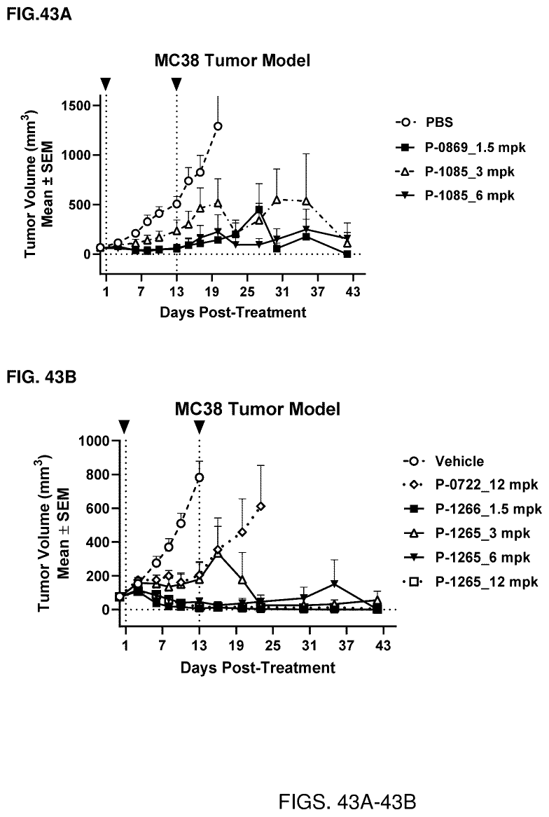

The anti-tumor efficacy of the PD1 Ab-IL-15 VitoKine, P-1265, relative to its dimeric VitoKine equivalent, P-1085, and their respective non-VitoKine counterparts, P-1266 and P-0869, was assessed in an established MC38 tumor model. All these compounds contain an IL-15 V63A/168H variant disputing its interaction with IL-15Rβ. P-1085 was administered two every 12 days (Q12D) dosages of 3 and 6 mg/kg, and P-0869, was given 2 doses of 1.5 mg/kg. The mean tumor volume, along with the standard error of the mean (SEM) for each group as a function of time, is illustrated in FIG. 43A. Mice treated with Vehicle rapidly developed large subcutaneous tumors, and other treatment groups all exhibited high efficacy in inhibiting tumor growth with close to 100% tumor growth inhibition (TGI) on 43 days after the start of the treatment.

P-1265, the PD1 Ab-IL15 VitoKine containing monomeric IL15 V63A/168H variant, was similarly compared to its active non-VitoKine counterpart, P-1266. On Day 1, treatments were given as follows: mouse PD1 antibody P-0722 at 12 mg/kg, P-1265 at varying dosing levels (3 mg/kg, 6 mg/kg, and 12 mg/kg), and P-1265 at 1.5 mg/kg. These treatments were given intraperitoneally Q12D for a total of 2 doses. Vehicle (PBS) was used as a control. As illustrated in FIG. 43B, P-0722 modestly delayed tumor growth, but all the other treatment groups exhibited high efficacy in tumor growth inhibition. Particularly for the VitoKine low dose group (3 mg/kg), the initial weaker tumor growth inhibition was reversed later and eventually resulted in 6 out of 7 mice that is free of tumor.

The anti-tumor activity of P-1263, a mouse PD1 Ab-IL-15 VitoKine containing monomeric IL-15 Q108N variant as the active domain, was assessed in an established MC38 model. P-1263 at varying dosing levels (6 mg/kg, 9 mg/kg, and 18 mg/kg) were administered twice following a Q12D schedule. The component PD1 antibody, P-0722, administered at dosages of 6 and 18 mg/kg, was included for comparative analysis.

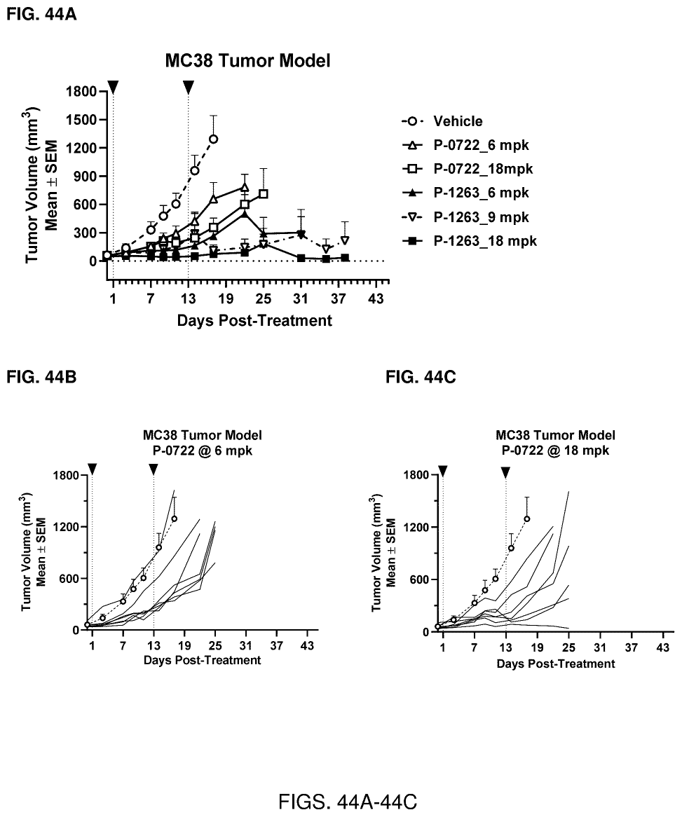

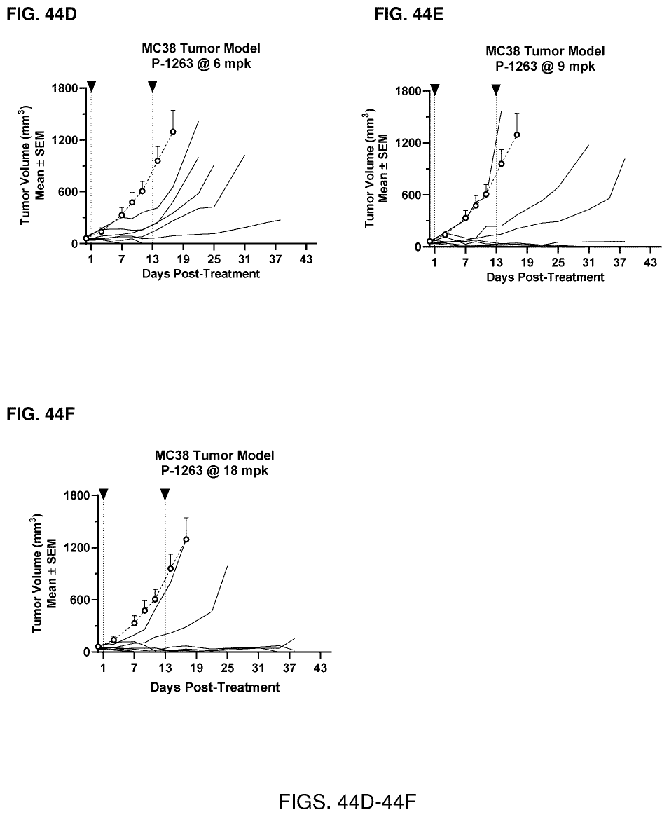

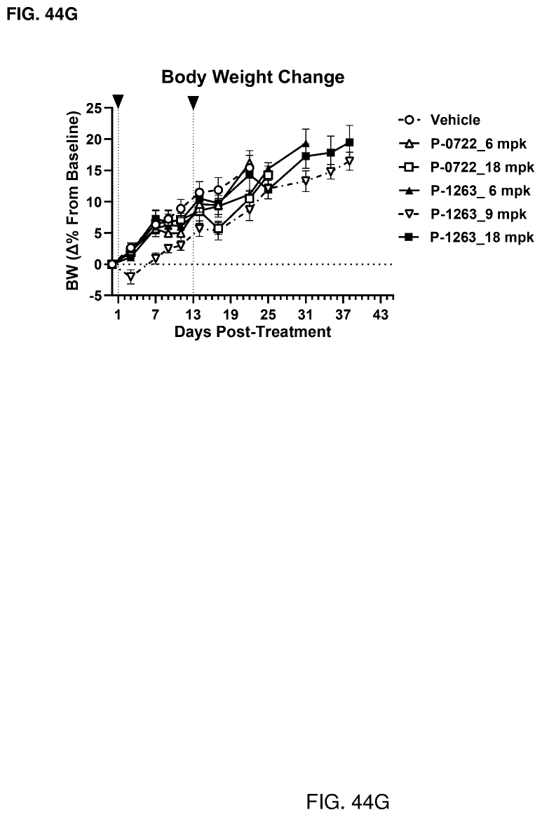

The mean tumor volume, along with SEM for each group as a function of time, is illustrated in FIG. 44A. Mice treated with Vehicle rapidly developed large subcutaneous tumors. The PD1 antibody treatment showed modest anti-tumor growth effect and such effect increased with the increasing dosage. Conversely, P-1263 treatment groups exhibited high efficacy in inhibiting tumor growth in a dose-dependent manner.

FIGS. 44B-44F further illustrate tumor growth curves of individual mice for the five different treatment groups. Each line in the graphs represents one mouse, along with the average tumor growth of the Vehicle group represented by a dotted line. The treatment with P-1263 at 18 mg/kg demonstrated the most pronounced and sustained effect, with 5 out of 7 mice from this group completely eradicating tumor growth on Day 38 (FIG. 44F). Likewise, in the group treated with P-1263 at 12 mg/kg, 4 out of 7 mice remained tumor-free at the end of study (FIG. 44E). On the other hand, P-1263 administered at 6 mg/kg had lower efficacy, with 2 out 7 mice remaining tumor-free (FIG. 44D). Other mice in each group showed tumor growth after an initial period of delayed tumor growth. For comparison, P-0722 dosed at 18 mg/kg did show some effect in delaying tumor growth, but the treatment did not result in total tumor eradication (FIG. 44C).

FIG. 44G shows that P-1263 was well tolerated with little or no body weight loss, even at dosages as high as 18 mg/kg. Notably, this anti-tumor efficacy was achieved with considerably lower peripheral lymphocyte proliferation and expansion (as seen in FIG. 40). This efficacy is partly attributed to the high dose tolerance afforded by the VitoKine format. Consequently, PD1 Ab-IL-15 VitoKine platform provides a wider therapeutic window, enabling the antibody component to fully achieve its potential in reversing T-cell anergy and exhaustion.

Taken together, PD1 Ab-IL-15 VitoKines effectively inhibited tumor growth while minimizing the proliferation and expansion of peripheral lymphocytes. Consequently, issues like over-stimulation of the immune pathway, undesirable “on-target” “off tissue” toxicity, and unwanted target sink generally associated with fully active cytokine could be mitigated by using the VitoKine format, without compromising the anti-tumor effectiveness. Importantly, the PD1 Ab-IL-15 VitoKine's compatibility with higher dosages ensures the antibody arm can optimally target and reverse T-cell anergy and exhaustion, potentiating existing immune responses. This will result in further enhancement of the immune system's activity against tumors.

As can be appreciated by skilled artisan, any PD1 Ab-IL-15 VitoKine construct comprising an optimized PD1 antibody, an IL-15 variant (dimeric or monomeric) with suitable potency to balance activity inertness before cleavage and potency after activation, and appropriate L1 and L2 linker sequences described herein come with the spirit and scope of the present invention.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Sequence Listings

The amino acid sequences listed in the accompanying sequence listing are shown using standard one letter codes for amino acids, as defined in 37 C.F.R. 1.822.

SEQ ID NO: 1 is the amino acid sequence of a mature human PD1 polypeptide.

SEQ ID NOS: 2-5 are the amino acid sequences of human PD1 blocking antibody light chain variable domains.

SEQ ID NOS: 6-18 are the amino acid sequences of human PD1 blocking antibody heavy chain variable domains.

SEQ ID NOS: 19-21 are the amino acid sequences of human PD1 blocking antibody light chain CDR1.

SEQ ID NOS: 22-24 are the amino acid sequences of human PD1 blocking antibody light chain CDR2.

SEQ ID NO: 25 is the amino acid sequence of human PD1 blocking antibody light chain CDR3.

SEQ ID NO: 26 is the amino acid sequence of human PD1 blocking antibody heavy chain CDR1.

SEQ ID NOS: 27-32 are the amino acid sequences of human PD1 blocking antibody heavy chain CDR2.

SEQ ID NO: 33 is the amino acid sequence of human PD1 blocking antibody heavy chain CDR3.

SEQ ID NO: 34 is the amino acid sequence of human kappa light chain constant domain.

SEQ ID NO: 35 is the amino acid sequence of human IgG1 heavy chain constant domain comprising L234A/L235A/G237A mutations.

SEQ ID NO: 36 is the amino acid sequence of human IgG4 heavy chain constant domain comprising S228P mutation.

SEQ ID NO: 37 is the amino acid sequence of human immunoglobulin germline exon HGHV1-2 (GenBank accession NO: X62106).

SEQ ID NO: 38 is the amino acid sequence of human immunoglobulin germline exon HGHV3-23 (GenBank accession NO: M99660).

SEQ ID NO: 39 is the amino acid sequence of human immunoglobulin germline exon HGKV3D-11 (GenBank accession NO: X17264).

SEQ ID NO: 40 is the amino acid sequence of human antibody heavy chain variable domain with GenBank accession NO: AB063829.

SEQ ID NO: 41 is the amino acid sequence of human antibody light chain variable domain with GenBank accession NO: M29469.

SEQ ID NO: 42 is the amino acid sequence of the light chain of reference human PD1 blocking antibody P-0734.

SEQ ID NO: 43 is the amino acid sequence of the heavy chain of reference human PD1 blocking antibody P-0734.

SEQ ID NO: 44 is the amino acid sequence of the light chain of human PD1 blocking antibodies.

SEQ ID NO: 45 is the amino acid sequence of the heavy chain of human PD1 blocking antibody P-1174.

SEQ ID NO: 46 is the amino acid sequence of the heavy chain of human PD1 blocking antibody P-1194.

SEQ ID NO: 47 is the amino acid sequence of the heavy chain of human PD1 blocking antibody P-1201.

SEQ ID NO: 48 is the amino acid sequence of the heavy chain of human PD1 blocking antibody P-1238.

SEQ ID NO: 49 is the amino acid sequence of the heavy chain of PD1 human blocking antibody P-1271.

SEQ ID NO: 50 is the amino acid sequence of the light chain of a benchmark human PD1 blocking antibody P-0795.

SEQ ID NO: 51 is the amino acid sequence of the heavy chain of a benchmark human PD1 blocking antibody P-0795.

SEQ ID NO: 52 is the amino acid sequence of the light chain of a surrogate mouse PD1 blocking antibody P-0722.

SEQ ID NO: 53 is the amino acid sequence of the heavy chain of a surrogate mouse PD1 blocking antibody P-0722.

SEQ ID SEQ ID NOS: 54-77 are the amino acid sequences of various protease substrate peptides.

SEQ ID NOS: 78-94 are the amino acid sequences of various protease cleavable linkers comprising various spacer peptides flanking protease substrate peptides.

SEQ ID NOS: 95-115 are the amino acid sequences of various non-cleavable linker sequences.

SEQ ID NO: 116 is a human IL-15 mature form amino acid sequence.

SEQ ID NOS: 117-163 are the amino acid sequences of human IL-15 variant polypeptides.

SEQ ID NO: 164 is a human IL-15Rα amino acid sequence.

SEQ ID NO: 165 is a human IL-15RαSushi domain+ amino acid sequence.

SEQ ID NO: 166 is the amino acid sequence of a human IgG1-Fc comprising L234A/L235A/G237A mutations.

SEQ ID NO: 167 is the amino acid sequence of a human IgG1 Knob-Fc comprising L234A/L235A/G237A mutations.

SEQ ID NO: 168 is the amino acid sequence of a human IgG1 Hole-Fc comprising L234A/L235A/G237A mutations.

SEQ ID NOs: 169 and 170 are the amino acid sequences of the heterodimeric heavy chain pair of a surrogate mouse PD1 blocking antibody P-0722.

SEQ ID NOS: 171 and 172 are the amino acid sequences of the heterodimeric heavy chains of a germline antibody P-1260.

SEQ ID NOS: 173 is the amino acid sequence of the light chain of a germline antibody P-1260.

SEQ ID NOS: 174 is the amino acid sequence of the PD1 human blocking antibody P-1271's heavy chain that contains the hole mutations.

SEQ ID NOS: 175-180 are the amino acid sequences of the heavy chains of various human PD1 Ab-IL-15 immunocytokines.

SEQ ID NOS: 181-185 are the amino acid sequences of the heavy chains of various human PD1 Ab-IL-15 VitoKines.

SEQUENCE LIST
Human PD1 protein mature sequence
(SEQ ID NO: 1)
FLDSPDRPWNPPTFSPALLVVTEGDNATFTCSFSNTSESFVLNWYRMSPSNQTDKLAAFP
EDRSQPGQDCRFRVTQLPNGRDFHMSVVRARRNDSGTYLCGAISLAPKAQIKESLRAELR
VTERRAEVPTAHPSPSPRPAGQFQTLVVGVVGGLLGSLVLLVWVLAVICSRAARGTIGAR
RTGQPLKEDPSAVPVFSVDYGELDFQWREKTPEPPVPCVPEQTEYATIVFPSGMGTSSPA
RRGSADGPRSAQPLRPEDGHCSWPL
Human PD1 blocking antibody light chain variable domain sequence
(SEQ ID NO: 2)
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKR
Human PD1 blocking antibody light chain variable domain
(SEQ ID NO: 3)
EIVLTQSPATLSLSPGERATLSCRASQGVSTSGYSYLHWYQQKPGQAPRLLIYLASYRES
GVPARFSGSGSGTDFTLTISSLEPEDFAVYYCQHSRDLPLTFGGGTKVEIKR
Human PD1 blocking antibody light chain variable domain sequence
(SEQ ID NO: 4)
EIVLTQSPATLSLSPGERATLSCRASQGVSTSGYSYLHWYQQKPGQAPRLLIYLASYRAS
GVPARFSGSGSGTDFTLTISSLEPEDFAVYYCQHSRDLPLTFGGGTKVEIKR
Human PD1 blocking antibody light chain variable domain sequence
(SEQ ID NO: 5)
EIVLTQSPATLSLSPGERATLSCRASQGVSTSGYSYLAWYQQKPGQAPRLLIYLASYRAS
GVPARFSGSGSGTDFTLTISSLEPEDFAVYYCQHSRDLPLTFGGGTKVEIKR
Human PD1 blocking antibody heavy chain variable domain sequence
(SEQ ID NO: 6)
QVQLVQSGVEVKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYWGQGTTVTVSS
Human PD1 blocking antibody heavy chain variable domain sequence
(SEQ ID NO: 7)
QVQLVQSGVEVKKPGASVKVSCKASGYTFTNYYMYWVRQAPGQGLEWMGGINPSNGGTNF
AQKFQGRVTLTTDSSTTTAYMELKSLQFDDTAVYYCARRDYRFDMGFDYWGQGTTVTVSS
Human PD1 blocking antibody heavy chain variable domain sequence
(SEQ ID NO: 8)
QVQLVQSGVEVKKPGASVKVSCKASGYTFTNYYMYWVRQAPGQGLEWMGGINPSNGGTNY
AQKFQGRVTLTTDSSTTTAYMELKSLQFDDTAVYYCARRDYRFDMGFDYWGQGTTVTVSS
Human PD1 blocking antibody heavy chain variable domain sequence
(SEQ ID NO: 9)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
Human PD1 blocking antibody heavy chain variable domain sequence
(SEQ ID NO: 10)
EVQLLESGGGLVQPGGSLRLSCAASGFTFTNYYMYWVRQAPGKGLEWVSGINPSNGGTNY
ADKFKGRFTISRDNSKNTLYLQMNSLRAEDTAVYYCARRDYRFDMGFDYWGQGTLVTVSS
Human PD1 blocking antibody heavy chain variable domain sequence
(SEQ ID NO: 11)
EVQLLESGGGLVQPGGSLRLSCAASGFTFTNYYMYWVRQAPGKGLEWVSGINPSNGGTNY
ADKFKGRFTLSTDSSKNTLYLQMNSLRAEDTAVYYCARRDYRFDMGFDYWGQGTLVTVSS
Human PD1 blocking antibody heavy chain variable domain sequence
(SEQ ID NO: 12)
EVQLLESGGGLVQPGGSLRLSCAASGFTFTNYYMYWVRQAPGKGLEWMGGINPSNGGTNY
ADKFKGRFTISRDNSKNTLYLQMNSLRAEDTAVYYCARRDYRFDMGFDYWGQGTLVTVSS
Human PD1 blocking antibody heavy chain variable domain sequence
(SEQ ID NO: 13)
EVQLLESGGGLVQPGGSLRLSCAASGFTFTNYYMYWVRQAPGKGLEWMGGINPSNGGTNY
ADKFKGRFTLSTDSSKNTLYLQMNSLRAEDTAVYYCARRDYRFDMGFDYWGQGTLVTVSS
Human PD1 blocking antibody heavy chain variable domain sequence
(SEQ ID NO: 14)
EVQLLESGGGLVQPGGSLRLSCAASGFTFTNYYMYWVRQAPGKGLEWVSGINPSNGGTNF
NDSVKGRFTISRDNSKNTLYLQMNSLRAEDTAVYYCARRDYRFDMGFDYWGQGTLVTVSS
Human PD1 blocking antibody heavy chain variable domain sequence
(SEQ ID NO: 15)
EVQLLESGGGLVQPGGSLRLSCAASGFTFTNYYMYWVRQAPGKGLEWVSGINPSNGGTNF
ADKFKGRFTISRDNSKNTLYLQMNSLRAEDTAVYYCARRDYRFDMGFDYWGQGTLVTVSS
Human PD1 blocking antibody heavy chain variable domain sequence
(SEQ ID NO: 16)
EVQLLESGGGLVQPGGSLRLSCAASGFTFTNYYMYWVRQAPGKGLEWVSGINPSNGGTNF
ADKFKGRFTISRDSSKNTLYLQMNSLRAEDTAVYYCARRDYRFDMGFDYWGQGTLVTVSS
Human PD1 blocking antibody heavy chain variable domain sequence
(SEQ ID NO: 17)
EVQLLESGGGLVQPGGSLRLSCAASGFTFTNYYMYWVRQAPGKGLEWVSGINPSNGGTNF
ADKFKGRFTISTDSSKNTLYLQMNSLRAEDTAVYYCARRDYRFDMGFDYWGQGTLVTVSS
Human PD1 blocking antibody heavy chain variable domain sequence
(SEQ ID NO: 18)
EVQLLESGGGLVQPGGSLRLSCAASGFTFTNYYMYWVRQAPGKGLEWVSGINPSNGGTNF
ADKFKGRFTLSTDSSKNTLYLQMNSLRAEDTAVYYCARRDYRFDMGFDYWGQGTLVTVSS
Human PD1 blocking antibody CDR-L1 sequence
(SEQ ID NO: 19)
RASKGVSTSGYSYLH
Human PD1 blocking antibody CDR-L1 sequence
(SEQ ID NO: 20)
RASQGVSTSGYSYLH
Human PD1 blocking antibody CDR-L1 sequence
(SEQ ID NO: 21)
RASQGVSTSGYSYLA
Human PD1 blocking antibody CDR-L2 sequence
(SEQ ID NO: 22)
YLASYLES
Human PD1 blocking antibody CDR-L2 sequence
(SEQ ID NO: 23)
YLASYRES
Human PD1 blocking antibody CDR-L2 sequence
(SEQ ID NO: 24)
YLASYRAS
Human PD1 blocking antibody CDR-L3 sequence
(SEQ ID NO: 25)
QHSRDLPLT
Human PD1 blocking antibody CDR-H1 sequence
(SEQ ID NO: 26)
NYYMY
Human PD1 blocking antibody CDR-H2 sequence
(SEQ ID NO: 27)
GINPSNGGTNFNEKFKN
Human PD1 blocking antibody CDR-H2 sequence
(SEQ ID NO: 28)
GINPSNGGTNFAQKFQG
Human PD1 blocking antibody CDR-H2 sequence
(SEQ ID NO: 29)
GINPSNGGTNYAQKFQG
Human PD1 blocking antibody CDR-H2 sequence
(SEQ ID NO: 30)
GINPSNGGTNYADKFKG
Human PD1 blocking antibody CDR-H2 sequence
(SEQ ID NO: 31)
GINPSNGGTNFADKFKG
Human PD1 blocking antibody CDR-H2 sequence
(SEQ ID NO: 32)
GINPSNGGTNFNDSVKG
Human PD1 blocking antibody CDR-H3 sequence
(SEQ ID NO: 33)
RDYRFDMGFDY
Human Kappa light chain constant domain sequence
(SEQ ID NO: 34)
TVAAPSVFIFPPSDEQLKSGTASVVCLLNNFYPREAKVQWKVDNALQSGNSQESVTEQDS
KDSTYSLSSTLTLSKADYEKHKVY ACEVTHQGLSSPVTKSFNRGEC
Human IgG1 constant domain with L234A/L235A/G237A mutations
sequence
(SEQ ID NO: 35)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Human IgG4 constant domain with S228P mutation sequence
(SEQ ID NO: 36)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLG
Human antibody germline IGHV1-2 sequence
(SEQ ID NO: 37)
QVQLVQSGAEVKKPGASVKVSCKASGYTFTGYYMHWVRQAPGQGLEWMGWINPNSGGTNY
AQKFQGRVTMTRDTSISTAYMELSRLRSDDTAVYYCAR
Human antibody germline IGHV3-23 sequence
(SEQ ID NO: 38)
EVQLLESGGGLVQPGGSLRLSCAASGFTFSSYAMSWVRQAPGKGLEWVSAISGSGGSTYY
ADSVKGRFTISRDNSKNTLYLQMNSLRA EDTAVYYCAK
Human antibody germline IGKV3D-11 sequence
(SEQ ID NO: 39)
EIVLTQSPATLSLSPGERATLSCRASQGVSSYLAWYQQKPGQAPRLLIYDASNRATGIPA
RFSGSGPGTDFTLTISSLEPEDFAVYYCQQRSNWH
Human antibody GenBank NO: AB063829 sequence
(SEQ ID NO: 40)
QVQLVQSGVEVKKPGASVKVSCKASGYTFTSNAISWVRQAPGQGLEWMGWISTYKGKANY
AQKFQDRVTLTTDSSTTTAYMELKSLQFDDTAVYYCARWRAVVGRGGGLDVWGQGTTVTV
SS
Human antibody GenBank NO: M29469 sequence
(SEQ ID NO: 41)
EIVLTQSPATLSLSPGERATLSCRASQSVSSYLAWYQQKPGQAPRLLIYDASNKATGVPA
RFSGSGSGTDFTLTISSLEPEDFAVYYCQQSSKWPLTFGGGTKVEIKG
Reference Antibody P-0734 light chain sequence
(SEQ ID NO: 42)
EIVLTQSPATLSLSPGERATLSCRASKGVSTSGYSYLHWYQQKPGQAPRLLIYLASYLES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQDSKDSTYSLS
STLTLSKADYEKHKVY ACEVTHQGLSSPVTKSFNRGEC
Reference Antibody P-0734 heavy chain sequence
(SEQ ID NO: 43)
QVQLVQSGVEVKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYWGQGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Human PD1 blocking Ab light chain sequence
(SEQ ID NO: 44)
EIVLTQSPATLSLSPGERATLSCRASQGVSTSGYSYLHWYQQKPGQAPRLLIYLASYRES
GVPARFSGSGSGTDFTLTISSLEPEDFAVYYCQHSRDLPLTFGGGTKVEIKRTVAAPSVF
IFPPSDEQLKSGTASVVCLLNNFYPREAKVQWKVDNALQSGNSQESVTEQDSKDSTYSLS
STLTLSKADYEKHKVY ACEVTHQGLSSPVTKSFNRGEC
Human PD1 blocking Ab P-1174 heavy chain sequence
(SEQ ID NO: 45)
QVQLVQSGVEVKKPGASVKVSCKASGYTFTNYYMYWVRQAPGQGLEWMGGINPSNGGTNF
AQKFQGRVTLTTDSSTTTAYMELKSLQFDDTAVYYCARRDYRFDMGFDYWGQGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Human PD1 blocking antibody P-1194 heavy chain sequence
(SEQ ID NO: 46)
EVQLLESGGGLVQPGGSLRLSCAASGFTFTNYYMYWVRQAPGKGLEWVSGINPSNGGTNY
ADKFKGRFTLSTDSSKNTLYLQMNSLRAEDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Human PD1 blocking antibody P-1201 heavy chain sequence
(SEQ ID NO: 47)
EVQLLESGGGLVQPGGSLRLSCAASGFTFTNYYMYWVRQAPGKGLEWVSGINPSNGGTNF
ADKFKGRFTLSTDSSKNTLYLQMNSLRAEDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Human PD1 blocking antibody P-1238 heavy chain sequence
(SEQ ID NO: 48)
EVQLLESGGGLVQPGGSLRLSCAASGFTFTNYYMYWVRQAPGKGLEWMGGINPSNGGTNY
ADKFKGRFTLSTDSSKNTLYLQMNSLRAEDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Human PD1 blocking Ab P-1271 heavy chain sequence
(SEQ ID NO: 49)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG
Benchmark human PD1 blocking antibody P-0795 light chain sequence
(SEQ ID NO: 50)
DIVMTQSPLSLPVTPGEPASITCKASQDVETVVAWYLQKPGQSPRLLIYWASTRHTGVPD
RFSGSGSGTDFTLKISRVEAEDVGVYYCQQYSRYPWTFGQGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Benchmark human PD1 blocking antibody P-0795 heavy chain sequence
(SEQ ID NO: 51)
EVQLVESGGGLVKPGGSLRLSCAASGFTFSSYDMSWVRQAPGKGLEWVATISGGGSYTYY
PDSVKGRFTISRDNAKNSLYLQMNSLRAEDTAVYYCASPDSSGVAYWGQGTLVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPG
Surrogate mouse PD1 block antibody P-0722 light chain sequence
(SEQ ID NO: 52)
DIVMTQGTLPNPVPSGESVSITCRSSKSLLYSDGKTYLNWYLQRPGQSPQLLIYWMSTRA
SGVSDRFSGSGSGTDFTLKISGVEAEDVGIYYCQQGLEFPTFGGGTKLELKRTDAAPTVS
IFPPSSEQLTSGGASVVCFLNNFYPRDINVKWKIDGSERQNGVLNSWTDQDSKDSTYSMS
STLTLTKDEYERHNSYTCEATHKTSTSPIVKSFNRNEC
Surrogate mouse PD1 block antibody P-0722 heavy chain sequence
(SEQ ID NO: 53)
EVQLQESGPGLVKPSQSLSLTCSVTGYSITSSYRWNWIRKFPGNRLEWMGYINSAGISNY
NPSLKRRISITRDTSKNQFFLQVNSVTTEDAATYYCARSDNMGTTPFTYWGQGTLVTVSS
AKTTPPSVYPLAPGSAAQTNSMVTLGCLVKGYFPEPVTVTWNSGSLSSGVHTFPAVLQSD
LYTLSSSVTVPSSTWPSQTVTCNVAHPASSTKVDKKIVPRDCGCKPCICTVPEVSSVFIF
PPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGGRPKAPQVYTIPPPKEQMAKDK
VSLTCMITNFFPEDITVEWQWNGQPAENYKNTQPIMDTDGSYFVYSKLNVQKSNWEAGNT
FTCSVLHEGLHNHHTEKSLSHSPG
Protease substrate peptide sequence
(SEQ ID NO: 54)
SPLGLAGS
Protease substrate peptide sequence
(SEQ ID NO: 55)
EPLELRAG
Protease substrate peptide sequence
(SEQ ID NO: 56)
LSGRSDNH
Protease substrate peptide sequence
(SEQ ID NO: 57)
GPLGIAGQ
Protease substrate peptide sequence
(SEQ ID NO: 58)
GTAHLMGG
Protease substrate peptide sequence
(SEQ ID NO: 59)
RIGSLRTA
Protease substrate peptide sequence
(SEQ ID NO: 60)
SGRSENIRTA
Protease substrate peptide sequence
(SEQ ID NO: 61)
GPLGMLSQ
Protease substrate peptide sequence
(SEQ ID NO: 62)
GPAGMKGL
Protease substrate peptide sequence
(SEQ ID NO: 63)
RPSASRSA
Protease substrate peptide sequence
(SEQ ID NO: 64)
PLGLAG
Protease substrate peptide sequence
(SEQ ID NO: 65)
LGGSGRSANAILE
Protease substrate peptide sequence
(SEQ ID NO: 66)
GGSGRSANAI
Protease substrate peptide sequence
(SEQ ID NO: 67)
SGRSA
Protease substrate peptide sequence
(SEQ ID NO: 68)
AANL
Protease substrate peptide sequence
(SEQ ID NO: 69)
GPTNKVR
Protease substrate peptide sequence
(SEQ ID NO: 70)
GFFY
Protease substrate peptide sequence
(SEQ ID NO: 71)
GPICFRLG
Protease substrate peptide sequence
(SEQ ID NO: 72)
RQAGFSL
Protease substrate peptide sequence
(SEQ ID NO: 73)
RQARAVGG
Protease substrate peptide sequence
(SEQ ID NO: 74)
PMAKK
Protease substrate peptide sequence
(SEQ ID NO: 75)
HSSKLQ
Protease substrate peptide sequence
(SEQ ID NO: 76)
GPLGMLSQPMAKK
Protease substrate peptide sequence
(SEQ ID NO: 77)
PMAKKGPLGMLSQ
Protease cleavable linker sequence
(SEQ ID NO: 78)
GGGSGGGGSGGGGSLSGRSDNHGGSGGGGS
Protease cleavable linker sequence
(SEQ ID NO: 79)
GSSSGRSENIRTAGT
Protease cleavable linker sequence
(SEQ ID NO: 80)
GGGGSGGGGSGGGSLGGSGRSANAILEGGSGGGGS
Protease cleavable linker sequence
(SEQ ID NO: 81)
GGGGSGGGGSLGGSGRSANAILEGGGGS
Protease cleavable linker sequence
(SEQ ID NO: 82)
GGGGSLGGSGRSANAILEGGS
Protease cleavable linker sequence
(SEQ ID NO: 83)
GGGSGPTNKVRGGS
Protease cleavable linker sequence
(SEQ ID NO: 84)
GGSGPLGMLSQGGGS
Protease cleavable linker sequence
(SEQ ID NO: 85)
GGPLGMLSQS
Protease cleavable linker sequence
(SEQ ID NO: 86)
GGGPLGMLSQGGS
Protease cleavable linker sequence
(SEQ ID NO: 87)
GGPTNKVRGS
Protease cleavable linker sequence
(SEQ ID NO: 88)
GRQARAVGGS
Protease cleavable linker sequence
(SEQ ID NO: 89)
GGGSGRSENIRTAGG
Protease cleavable linker sequence
(SEQ ID NO: 90)
SGGPGPAGMKGLPGS
Protease cleavable linker sequence
(SEQ ID NO: 91)
GGGGSPMAKKGGGGS
Protease cleavable linker sequence
(SEQ ID NO: 92)
GGPLGMLSQPMAKKS
Protease cleavable linker sequence
(SEQ ID NO: 93)
GGSGPLGMLSQPMAKKGGGS
Protease cleavable linker sequence
(SEQ ID NO: 94)
GGGPMAKKGPLGMLSQGGGS
Non-cleavable linker sequence
(SEQ ID NO: 95)
EPKSSDKTHTSPPS
Non-cleavable linker sequence
(SEQ ID NO: 96)
GGGSGGGSGGGS
Non-cleavable linker sequence
(SEQ ID NO: 97)
GGGS
Non-cleavable linker sequence
(SEQ ID NO: 98)
GSSGGSGGS
Non-cleavable linker sequence
(SEQ ID NO: 99)
GSSGT
Non-cleavable linker sequence
(SEQ ID NO: 100)
GGGGSGGGGSGGGS
Non-cleavable linker sequence
(SEQ ID NO: 101)
AEAAAKEAAAKEAAAKA
Non-cleavable linker sequence
(SEQ ID NO: 102)
GGGGSGGGGSGGGGSGGGGS
Non-cleavable linker sequence
(SEQ ID NO: 103)
GGGSGGGS
Non-cleavable linker sequence
(SEQ ID NO: 104)
GS
Non-cleavable linker sequence
(SEQ ID NO: 105)
GGS
Non-cleavable linker sequence
(SEQ ID NO: 106)
GGGGS
Non-cleavable linker sequence
(SEQ ID NO: 107)
GGSGG
Non-cleavable linker sequence
(SEQ ID NO: 108)
SGGG
Non-cleavable linker sequence
(SEQ ID NO: 109)
GSGS
Non-cleavable linker sequence
(SEQ ID NO: 110)
GSGSGS
Non-cleavable linker sequence
(SEQ ID NO: 111)
GSGSGSGS
Non-cleavable linker sequence
(SEQ ID NO: 112)
GSGSGSGSGS
Non-cleavable linker sequence
(SEQ ID NO: 113)
GSGSGSGSGSGS
Non-cleavable linker sequence
(SEQ ID NO: 114)
GGGGSGGGGS
Non-cleavable linker sequence
(SEQ ID NO: 115)
GGGGSGGGGSGGGGS
Human IL-15 mature form sequence
(SEQ ID NO: 116)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFINTS
Human IL-15 S58D variant sequence
(SEQ ID NO: 117)
NWVNVISDLKKIEDLIQSMHIDATLYTESDVHPSCKVTAMKCFLLELQVISLESGDADIH
DTVENLIILANNSLSSNGNVTESGCKECEELEEKNIKEFLQSFVHIVQMFINTS
Human IL-15 variant with 1 amino acid deletion at the N-terminus
(SEQ ID NO: 118)
WVNVISDLKKIEDLIQSMHIDATLYTESDVHPSCKVTAMKCFLLELQVISLESGDASIHD
TVENLIILANNSLSSNGNVTESGCKECEELEEKNIKEFLQSFVHIVQMFINTS
Human IL-15 variant with 2 amino acid deletion at the N-terminus
(SEQ ID NO: 119)
VNVISDLKKIEDLIQSMHIDATLYTESDVHPSCKVTAMKCFLLELQVISLESGDASIHDT
VENLIILANNSLSSNGNVTESGCKECEELEEKNIKEFLQSFVHIVQMFINTS
Human IL-15 variant with 3 amino acid deletion at the N-terminus
(SEQ ID NO: 120)
NVISDLKKIEDLIQSMHIDATLYTESDVHPSCKVTAMKCFLLELQVISLESGDASIHDTV
ENLIILANNSLSSNGNVTESGCKECEELEEKNIKEFLQSFVHIVQMFINTS
Human IL-15 V63A variant sequence
(SEQ ID NO: 121)
NWVNVISDLKKIEDLIQSMHIDATLYTESDVHPSCKVTAMKCFLLELQVISLESGDASIH
DTAENLIILANNSLSSNGNVTESGCKECEELEEKNIKEFLQSFVHIVQMFINTS
Human IL-15 V63F variant sequence
(SEQ ID NO: 122)
NWVNVISDLKKIEDLIQSMHIDATLYTESDVHPSCKVTAMKCFLLELQVISLESGDASIH
DTFENLIILANNSLSSNGNVTESGCKECEELEEKNIKEFLQSFVHIVQMFINTS
Human IL-15 V63K variant sequence
(SEQ ID NO: 123)
NWVNVISDLKKIEDLIQSMHIDATLYTESDVHPSCKVTAMKCFLLELQVISLESGDASIH
DTKENLIILANNSLSSNGNVTESGCKECEELEEKNIKEFLQSFVHIVQMFINTS
Human IL-15 V63R variant sequence
(SEQ ID NO: 124)
NWVNVISDLKKIEDLIQSMHIDATLYTESDVHPSCKVTAMKCFLLELQVISLESGDASIH
DTRENLIILANNSLSSNGNVTESGCKECEELEEKNIKEFLQSFVHIVQMFINTS
Human IL-15 168D variant sequence
(SEQ ID NO: 125)
NWVNVISDLKKIEDLIQSMHIDATLYTESDVHPSCKVTAMKCFLLELQVISLESGDASIH
DTVENLIDLANNSLSSNGNVTESGCKECEELEEKNIKEFLQSFVHIVQMFINTS
Human IL-15 168F variant sequence
(SEQ ID NO: 126)
NWVNVISDLKKIEDLIQSMHIDATLYTESDVHPSCKVTAMKCFLLELQVISLESGDASIH
DTVENLIFLANNSLSSNGNVTESGCKECEELEEKNIKEFLQSFVHIVQMFINTS
Human IL-15 168G variant sequence
(SEQ ID NO: 127)
NWVNVISDLKKIEDLIQSMHIDATLYTESDVHPSCKVTAMKCFLLELQVISLESGDASIH
DTVENLIGLANNSLSSNGNVTESGCKECEELEEKNIKEFLQSFVHIVQMFINTS
Human IL-15 168H variant sequence
(SEQ ID NO: 128)
NWVNVISDLKKIEDLIQSMHIDATLYTESDVHPSCKVTAMKCFLLELQVISLESGDASIH
DTVENLIHLANNSLSSNGNVTESGCKECEELEEKNIKEFLQSFVHIVQMFINTS
Human IL-15 168K variant sequence
(SEQ ID NO: 129)
NWVNVISDLKKIEDLIQSMHIDATLYTESDVHPSCKVTAMKCFLLELQVISLESGDASIH
DTVENLIKLANNSLSSNGNVTESGCKECEELEEKNIKEFLQSFVHIVQMFINTS
Human IL-15 168Q variant sequence
(SEQ ID NO: 130)
NWVNVISDLKKIEDLIQSMHIDATLYTESDVHPSCKVTAMKCFLLELQVISLESGDASIH
DTVENLIQLANNSLSSNGNVTESGCKECEELEEKNIKEFLQSFVHIVQMFINTS
Human IL-15 V63A/168G variant sequence
(SEQ ID NO: 131)
NWVNVISDLKKIEDLIQSMHIDATLYTESDVHPSCKVTAMKCFLLELQVISLESGDASIH
DTAENLIGLANNSLSSNGNVTESGCKECEELEEKNIKEFLQSFVHIVQMFINTS
Human IL-15 V63A/168H variant sequence
(SEQ ID NO: 132)
NWVNVISDLKKIEDLIQSMHIDATLYTESDVHPSCKVTAMKCFLLELQVISLESGDASIH
DTAENLIHLANNSLSSNGNVTESGCKECEELEEKNIKEFLQSFVHIVQMFINTS
Human IL-15 V63A/168Q variant sequence
(SEQ ID NO: 133)
NWVNVISDLKKIEDLIQSMHIDATLYTESDVHPSCKVTAMKCFLLELQVISLESGDASIH
DTAENLIQLANNSLSSNGNVTESGCKECEELEEKNIKEFLQSFVHIVQMFINTS
Human IL-15 Q108A variant sequence
(SEQ ID NO: 134)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVAMFINTS
Human IL-15 Q108D variant sequence
(SEQ ID NO: 135)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVDMFINTS
Human IL-15 Q108E variant sequence
(SEQ ID NO: 136)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVEMFINTS
Human IL-15 Q108F variant sequence
(SEQ ID NO: 137)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVFMFINTS
Human IL-15 Q108H variant sequence
(SEQ ID NO: 138)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVHMFINTS
Human IL-15 Q108K variant sequence
(SEQ ID NO: 139)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVKMFINTS
Human IL-15 Q108L variant sequence
(SEQ ID NO: 140)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVLMFINTS
Human IL-15 Q108M variant sequence
(SEQ ID NO: 141)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVMMFINTS
Human IL-15 Q108N variant sequence
(SEQ ID NO: 142)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVNMFINTS
Human IL-15 Q108S variant sequence
(SEQ ID NO: 143)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVSMFINTS
Human IL-15 Q108T variant sequence
(SEQ ID NO: 144)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVTMFINTS
Human IL-15 Q108Y variant sequence
(SEQ ID NO: 145)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVYMFINTS
Human IL-15 V63A/Q108M variant sequence
(SEQ ID NO: 146)
NWVNVISDLKKIEDLIQSMHIDATLYTESDVHPSCKVTAMKCFLLELQVISLESGDASIH
DTAENLIILANNSLSSNGNVTESGCKECEELEEKNIKEFLQSFVHIVMMFINTS
Human IL-15 V63K/Q108M variant sequence
(SEQ ID NO: 147)
NWVNVISDLKKIEDLIQSMHIDATLYTESDVHPSCKVTAMKCFLLELQVISLESGDASIH
DTKENLIILANNSLSSNGNVTESGCKECEELEEKNIKEFLQSFVHIVMMFINTS
Human IL-15 168F/Q108M variant sequence
(SEQ ID NO: 148)
NWVNVISDLKKIEDLIQSMHIDATLYTESDVHPSCKVTAMKCFLLELQVISLESGDASIH
DTVENLIFLANNSLSSNGNVTESGCKECEELEEKNIKEFLQSFVHIVMMFINTS
Human IL-15 168H/Q108M variant sequence
(SEQ ID NO: 149)
NWVNVISDLKKIEDLIQSMHIDATLYTESDVHPSCKVTAMKCFLLELQVISLESGDASIH
DTVENLIHLANNSLSSNGNVTESGCKECEELEEKNIKEFLQSFVHIVMMFINTS
Human IL-15 V63K/Q108N variant sequence
(SEQ ID NO: 150)
NWVNVISDLKKIEDLIQSMHIDATLYTESDVHPSCKVTAMKCFLLELQVISLESGDASIH
DTKENLIILANNSLSSNGNVTESGCKECEELEEKNIKEFLQSFVHIVNMFINTS
Human IL-15 168H/Q108N variant sequence
(SEQ ID NO: 151)
NWVNVISDLKKIEDLIQSMHIDATLYTESDVHPSCKVTAMKCFLLELQVISLESGDASIH
DTVENLIHLANNSLSSNGNVTESGCKECEELEEKNIKEFLQSFVHIVNMFINTS
Human IL-15 D30T variant sequence
(SEQ ID NO: 152)
NWVNVISDLKKIEDLIQSMHIDATLYTESTVHPSCKVTAMKCFLLELQVISLESGDASIH
DTVENLIILANNSLSSNGNVTESGCKECEELEEKNIKEFLQSFVHIVQMFINTS
Human IL-15 H32E variant sequence
(SEQ ID NO: 153)
NWVNVISDLKKIEDLIQSMHIDATLYTESDVEPSCKVTAMKCFLLELQVISLESGDASIH
DTVENLIILANNSLSSNGNVTESGCKECEELEEKNIKEFLQSFVHIVQMFINTS
Human IL-15 H32D variant sequence
(SEQ ID NO: 154)
NWVNVISDLKKIEDLIQSMHIDATLYTESDVDPSCKVTAMKCFLLELQVISLESGDASIH
DTVENLIILANNSLSSNGNVTESGCKECEELEEKNIKEFLQSFVHIVQMFINTS
Human IL-15 H32N variant sequence
(SEQ ID NO: 155)
NWVNVISDLKKIEDLIQSMHIDATLYTESDVNPSCKVTAMKCFLLELQVISLESGDASIH
DTVENLIILANNSLSSNGNVTESGCKECEELEEKNIKEFLQSFVHIVQMFINTS
Human IL-15 H32Q variant sequence
(SEQ ID NO: 156)
NWVNVISDLKKIEDLIQSMHIDATLYTESDVQPSCKVTAMKCFLLELQVISLESGDASIH
DTVENLIILANNSLSSNGNVTESGCKECEELEEKNIKEFLQSFVHIVQMFINTS
Human IL-15 M109A variant sequence
(SEQ ID NO: 157)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQAFINTS
Human IL-15 M109H variant sequence
(SEQ ID NO: 158)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQHFINTS
Human IL-15 M109R variant sequence
(SEQ ID NO: 159)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQRFINTS
Human IL-15 N112D variant sequence
(SEQ ID NO: 160)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFIDTS
Human IL-15 N112G variant sequence
(SEQ ID NO: 161)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFIGTS
Human IL-15 N112P variant sequence
(SEQ ID NO: 162)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFIPTS
Human IL-15 N112R variant sequence
(SEQ ID NO: 163)
NWVNVISDLKKIEDLIQSMHIDATLYTESDVHPSCKVTAMKCFLLELQVISLESGDASIH
DTVENLIILANNSLSSNGNVTESGCKECEELEEKNIKEFLQSFVHIVQMFIRTS
Human IL-15Rα polypeptide sequence
(SEQ ID NO: 164)
MAPRRARGCRTLGLPALLLLLLLRPPATRGITCPPPMSVEHADIWVKSYSLYSRERYICN
SGFKRKAGTSSLTECVLNKATNVAHWTTPSLKCIRDPALVHQRPAPPSTVTTAGVTPQPE
SLSPSGKEPAASSPSSNNTAATTAAIVPGSQLMPSKSPSTGTTEISSHESSHGTPSQTTA
KNWELTASASHQPPGVYPQGHSDTTVAISTSTVLLCGLSAVSLLACYLKSRQTPPLASVE
MEAMEALPVTWGTSSRDEDLENCSHHL
Human IL-15Rαsushi+ domain sequence
(SEQ ID NO: 165)
ITCPPPMSVEHADIWVKSYSLYSRERYICNSGFKRKAGTSSLTECVLNKATNVAHWTTPS
LKCIRDPALVHQRPAPP
Human IgG1-Fc comprising L234A/L235A/G237A mutations sequence
(SEQ ID NO: 166)
DKTHTCPPCPAPEAAGAPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG
Human IgG1 Fc knob chain comprising L234A/L235A/G237A
mutations sequence
(SEQ ID NO: 167)
DKTHTCPPCPAPEAAGAPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVCTLPPSREEMTKNQVSLWCLVKGFYPSDIAVEWESNGQPENNYKTTPPVLDS
DGSFFLYSKLTVDKSRWQQGNVFSCSVMHEALHNHYTQKSLSLSPG
Human IgG1 Fc hole chain comprising L234A/L235A/G237A
mutations sequence
(SEQ ID NO: 168)
DKTHTCPPCPAPEAAGAPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPCREEMTKNQVSLSCAVKGFYPSDIAVEWESNGQPENNYKTTPPVLDS
DGSFFLVSKLTVDKSRWQQGNVFSCSVMHEALHNHYTQKSLSLSPG
Surrogate mouse PD1 Ab P-0722 heterodimeric heavy chain 1
sequence
(SEQ ID NO: 169)
EVQLQESGPGLVKPSQSLSLTCSVTGYSITSSYRWNWIRKFPGNRLEWMGYINSAGISNY
NPSLKRRISITRDTSKNQFFLQVNSVTTEDAATYYCARSDNMGTTPFTYWGQGTLVTVSS
AKTTPPSVYPLAPGSAAQTNSMVTLGCLVKGYFPEPVTVTWNSGSLSSGVHTFPAVLQSD
LYTLSSSVTVPSSTWPSQTVTCNVAHPASSTKVDKKIVPRDCGCKPCICTVPEVSSVFIF
PPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKEQMAKDKV
SLTCMITNFFPEDITVEWQWNGQPAENYDNTQPIMDTDGSYFVYSDLNVQKSNWEAGNTF
TCSVLHEGLHNHHTEKSLSHSPG
Surrogate mouse PD1 Ab P-0722 heterodimeric heavy chain 2
sequence
(SEQ ID NO: 170)
EVQLQESGPGLVKPSQSLSLTCSVTGYSITSSYRWNWIRKFPGNRLEWMGYINSAGISNY
NPSLKRRISITRDTSKNQFFLQVNSVTTEDAATYYCARSDNMGTTPFTYWGQGTLVTVSS
AKTTPPSVYPLAPGSAAQTNSMVTLGCLVKGYFPEPVTVTWNSGSLSSGVHTFPAVLQSD
LYTLSSSVTVPSSTWPSQTVTCNVAHPASSTKVDKKIVPRDCGCKPCICTVPEVSSVFIF
PPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKKQMAKDKV
SLTCMITNFFPEDITVEWQWNGQPAENYKNTQPIMKTDGSYFVYSKLNVQKSNWEAGNTF
TCSVLHEGLHNHHTEKSLSHSPG
Germline antibody P-1260 heterodimeric heavy chain 1
(with knob mutations) sequence
(SEQ ID NO: 171)
EVQLLESGGGLVQPGGSLRLSCAASGFTFSSYAMSWVRQAPGKGLEWVSAISGSGGSTYY
ADSVKGRFTISRDNSKNTLYLQMNSLRAEDTAVYYCAKWDGDYWGQGTLVTVSSASTKGP
SVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPEAAGAPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPCREEMTKNQV
SLWCLVKGFYPSDIAVEWESNGQPENNYKTTPPVLDSDGSFFLYSKLTVDKSRWQQGNVF
SCSVMHEALHNHYTQKSLSLSPG
Germline antibody P-1260 heterodimeric heavy chain 2
(with hole mutations) sequence
(SEQ ID NO: 172)
EVQLLESGGGLVQPGGSLRLSCAASGFTFSSYAMSWVRQAPGKGLEWVSAISGSGGSTYY
ADSVKGRFTISRDNSKNTLYLQMNSLRAEDTAVYYCAKWDGDYWGQGTLVTVSSASTKGP
SVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPEAAGAPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVCTLPPSREEMTKNQV
SLSCAVKGFYPSDIAVEWESNGQPENNYKTTPPVLDSDGSFFLVSKLTVDKSRWQQGNVF
SCSVMHEALHNHYTQKSLSLSPG
Germline antibody P-1260 light chain sequence
(SEQ ID NO: 173)
EIVLTQSPGTLSLSPGERATLSCRASQSVSSSYLAWYQQKPGQAPRLLIYGASSRATGIP
DRFSGSGSGTDFTLTISRLEPEDFAVYYCQQYGSSPYTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Human PD1 Ab P-1271 heavy chain with hole mutations sequence
(SEQ ID NO: 174)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVCTLPPSREE
MTKNQVSLSCAVKGFYPSDIAVEWESNGQPENNYKTTPPVLDSDGSFFLVSKLTVDKSRW
QQGNVFSCSVMHEALHNHYTQKSLSLSPG
Human PD1 Ab-IL-15 immunocytokine P-0870 heavy chain sequence
(SEQ ID NO: 175)
EVQLVESGGGLVKPGGSLRLSCAASGFTFSSYDMSWVRQAPGKGLEWVATISGGGSYTYY
PDSVKGRFTISRDNAKNSLYLQMNSLRAEDTAVYYCASPDSSGVAYWGQGTLVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGGGGGSGGGGSGGGGSNWVNVISDLKKIEDLIQSM
HIDATLYTESDVHPSCKVTAMKCFLLELQVISLESGDASIHDTAENLIHLANNSLSSNGN
VTESGCKECEELEEKNIKEFLQSFVHIVQMFINTS
Human PD1 Ab-IL-15 immunocytokine P-0886 heavy chain sequence
(SEQ ID NO: 176)
EVQLVESGGGLVKPGGSLRLSCAASGFTFSSYDMSWVRQAPGKGLEWVATISGGGSYTYY
PDSVKGRFTISRDNAKNSLYLQMNSLRAEDTAVYYCASPDSSGVAYWGQGTLVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGGGGGSGGGGSGGGGSVNVISDLKKIEDLIQSMHI
DATLYTESDVHPSCKVTAMKCFLLELQVISLESGDASIHDTVENLIILANNSLSSNGNVT
ESGCKECEELEEKNIKEFLQSFVHIVQMFINTS
Human PD1 Ab-IL-15 immunocytokine P-1369 heavy chain sequence
(SEQ ID NO: 177)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTAENLIHLANNSLSS
NGNVTESGCKECEELEEKNIKEFLQSFVHIVQMFINTS
Human PD1 Ab-IL-15 immunocytokine P-1385 heavy chain 1 sequence
(SEQ ID NO: 178)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTAENLIHLANNSLSS
NGNVTESGCKECEELEEKNIKEFLQSFVHIVQMFINTS
Human PD1 Ab-IL-15 immunocytokine P-1380 heavy chain 1 sequence
(SEQ ID NO: 179)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTVENLIILANNSLSS
NGNVTESGCKECEELEEKNIKEFLQSFVHIVNMFINTS
Human PD1 Ab-IL-15 immunocytokine P-1352 heavy chain 1 sequence
(SEQ ID NO: 180)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTVENLIHLANNSLSS
NGNVTESGCKECEELEEKNIKEFLQSFVHIVNMFINTS
Human PD1 Ab-IL-15 VitoKine P-0875 heavy chain sequence
(SEQ ID NO: 181)
EVQLVESGGGLVKPGGSLRLSCAASGFTFSSYDMSWVRQAPGKGLEWVATISGGGSYTYY
PDSVKGRFTISRDNAKNSLYLQMNSLRAEDTAVYYCASPDSSGVAYWGQGTLVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGGGGGSGGGGSGGGGSNWVNVISDLKKIEDLIQSM
HIDATLYTESDVHPSCKVTAMKCFLLELQVISLESGDASIHDTAENLIHLANNSLSSNGN
VTESGCKECEELEEKNIKEFLQSFVHIVQMFINTSGGPLGMLSQSITCPPPMSVEHADIW
VKSYSLYSRERYICNSGFKRKAGTSSLTECVLNKATNVAHWTTPSLKCIRDPALVHQRPA
PP
Human PD1 Ab-IL-15 VitoKine P-1349 heavy chain sequence
(SEQ ID NO: 182)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTAENLIHLANNSLSS
NGNVTESGCKECEELEEKNIKEFLQSFVHIVQMFINTSGGSGPLGMLSQPMAKKGGGSIT
CPPPMSVEHADIWVKSYSLYSRERYICNSGFKRKAGTSSLTECVLNKATNVAHWTTPSLK
CIRDPALVHQRPAPP
Human PD1 Ab-IL-15 VitoKine P-1339 heavy chain 1 sequence
(SEQ ID NO: 183)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTAENLIHLANNSLSS
NGNVTESGCKECEELEEKNIKEFLQSFVHIVQMFINTSGGSGPLGMLSQPMAKKGGGSIT
CPPPMSVEHADIWVKSYSLYSRERYICNSGFKRKAGTSSLTECVLNKATNVAHWTTPSLK
CIRDPALVHQRPAPP
Human PD1 Ab-IL-15 VitoKine P-1340 heavy chain 1 sequence
(SEQ ID NO: 184)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTVENLIILANNSLSS
NGNVTESGCKECEELEEKNIKEFLQSFVHIVNMFINTSGGSGPLGMLSQPMAKKGGGSIT
CPPPMSVEHADIWVKSYSLYSRERYICNSGFKRKAGTSSLTECVLNKATNVAHWTTPSLK
CIRDPALVHQRPAPP
Human PD1 Ab-IL-15 VitoKine P-1350 heavy chain sequence
(SEQ ID NO: 185)
QVQLVQSGAEVKKPGASVKVSCKASGYTFTNYYMYWVRQAPGQGLEWMGGINPSNGGTNF
AQKFQGRVTLTTDSSTSTAYMELSSLRSDDTAVYYCARRDYRFDMGFDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGA
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGGGGGSGGGGSGGGGSNWVNVISDLKKIEDLI
QSMHIDATLYTESDVHPSCKVTAMKCFLLELQVISLESGDASIHDTVENLIILANNSLSS
NGNVTESGCKECEELEEKNIKEFLQSFVHIVNMFINTSGGSGPLGMLSQPMAKKGGGSIT
CPPPMSVEHADIWVKSYSLYSRERYICNSGFKRKAGTSSLTECVLNKATNVAHWTTPSLK
CIRDPALVHQRPAPP

1-37. (canceled)

38. An isolated Interleukin-15 (IL-15) fusion protein complex comprising: (1) an IL-15 polypeptide (or variant thereof) linked to an PD1 blocking antibody; and (2) an IL-15 Receptor alpha (“IL-15Rα”) domain noncovalently linked to the IL-15 polypeptide to form an IL-15/IL-15Rα-PD1 blocking antibody fusion protein, wherein the PD1 blocking antibody is selected from an antibody which comprises: (a) a light chain variable region comprising amino acids having the sequence set forth in SEQ ID NO: 3, and a heavy chain variable region comprising amino acids having the sequence set forth in SEQ ID NO: 7; or (b) a light chain variable region comprising amino acids having the sequence set forth in SEQ ID NO: 3, and a heavy chain variable region comprising amino acids having the sequence set forth in SEQ ID NO: 9; or (c) a light chain variable region comprising amino acids having the sequence set forth in SEQ ID NO: 3, and a heavy chain variable region comprising amino acids having the sequence set forth in SEQ ID NO: 11; (d) a light chain variable region comprising amino acids having the sequence set forth in SEQ ID NO: 3, and a heavy chain variable region comprising amino acids having the sequence set forth in SEQ ID NO: 13; or (e) a light chain variable region comprising amino acids having the sequence set forth in SEQ ID NO: 3, and a heavy chain variable region comprising amino acids having the sequence set forth in SEQ ID NO: 18, and wherein the PD1 bocking antibody targets the IL-15/IL-15Rα-PD1 antibody fusion protein to tumor-infiltrating lymphocytes (TILs).

39. The IL-15/IL-15Rα-PD1 blocking antibody fusion protein according to claim 38, wherein the PD1 blocking antibody is selected from an antibody comprising: (a) the light chain sequence set forth in SEQ ID NO: 44, and the heavy chain sequence set forth in SEQ ID NO: 45; or comprising (b) the light chain sequence set forth in SEQ ID NO: 44, and the heavy chain sequence set forth in SEQ ID NO: 46; or comprising (c) the light chain sequence set forth in SEQ ID NO: 44, and the heavy chain sequence set forth in SEQ ID NO: 47; or the light chain sequence set forth in SEQ ID NO: 44; or comprising (d) the heavy chain sequence set forth in SEQ ID NO: 48; or comprising (e) the light chain sequence set forth in SEQ ID NO: 44, and the heavy chain sequence set forth in SEQ ID NO: 49.

40. The IL-15/IL-15Rα-PD1 blocking antibody fusion protein according to claim 38, wherein the IL-15 polypeptide is linked to the C-terminus of the PD1 blocking antibody.

41. The IL-15/IL-15Rα-PD1 blocking antibody fusion protein according to claim 38, wherein the IL-15 variant polypeptide is selected from the group of polypeptides having the amino acid sequence set forth in SEQ ID NOs: 117-163.

42. The IL-15/IL-15Rα-PD1 blocking antibody fusion protein according to claim 38, wherein the IL-15Rα domain comprises the amino acid sequence set forth in SEQ ID NO: 165 or any functional fragment thereof.

43. The IL-15/IL-15Rα-PD1 blocking antibody fusion protein according to claim 38, wherein the IL-15 polypeptide is covalently attached to the PD1 blocking antibody by a peptide linker, wherein the peptide linker is selected from the group of sequences set forth in SEQ ID NOs: 54-115.

44. The IL-15/IL-15Rα-PD1 blocking antibody fusion protein according to claim 38, wherein the fusion protein is selected from the group of fusion proteins wherein (a) the fusion protein is in the form of a monomer, or (2) the fusion protein is in the form of a dimer.

45. A pharmaceutical composition comprising a fusion protein according to claim 38 in admixture with a pharmaceutically acceptable carrier.

46. A method for treating cancer or cancer metastasis in a subject, comprising administering to the subject a therapeutically effective amount of the pharmaceutical composition according to claim 45.

47. The method according to claim 46, wherein the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.

48. A bioactivatable polypeptide drug construct comprising, in an N- to C-terminal direction (D1-D2-D3): 1) a tumor-infiltrating lymphocyte (TIL)-targeting moiety D1 domain (“D1”), 2) a bioactivatable moiety D2 domain (“D2”), and 3) a concealing moiety D3 domain (“D3”); wherein D1 functions to target the bioactivatable moiety to the intended site of therapy; wherein D3 is capable of concealing the functional activity of D2 until activated at the intended site of therapy; wherein D1 is an PD1 blocking antibody, wherein D2 is an IL-15 variant polypeptide, and wherein D3 is an IL-15Rα domain.

49. The bioactivatable polypeptide drug construct according to claim 48, wherein the PD1 blocking antibody is selected from an antibody which comprises: (a) a light chain variable region comprising amino acids having the sequence set forth in SEQ ID NO: 3, and a heavy chain variable region comprising amino acids having the sequence set forth in SEQ ID NO: 7; or (b) a light chain variable region comprising amino acids having the sequence set forth in SEQ ID NO: 3, and a heavy chain variable region comprising amino acids having the sequence set forth in SEQ ID NO: 9; or (c) a light chain variable region comprising amino acids having the sequence set forth in SEQ ID NO: 3, and a heavy chain variable region comprising amino acids having the sequence set forth in SEQ ID NO: 11; (d) a light chain variable region comprising amino acids having the sequence set forth in SEQ ID NO: 3, and a heavy chain variable region comprising amino acids having the sequence set forth in SEQ ID NO: 13; or (e) a light chain variable region comprising amino acids having the sequence set forth in SEQ ID NO: 3, and a heavy chain variable region comprising amino acids having the sequence set forth in SEQ ID NO: 18.

50. The bioactivatable polypeptide drug construct according to claim 48, wherein the PD1 blocking antibody is selected from an antibody comprising: (a) the light chain sequence set forth in SEQ ID NO: 44, and the heavy chain sequence set forth in SEQ ID NO: 45; or comprising (b) the light chain sequence set forth in SEQ ID NO: 44, and the heavy chain sequence set forth in SEQ ID NO: 46; or comprising (c) the light chain sequence set forth in SEQ ID NO: 44, and the heavy chain sequence set forth in SEQ ID NO: 47; or the light chain sequence set forth in SEQ ID NO: 44; or comprising (d) the heavy chain sequence set forth in SEQ ID NO: 48; or comprising (e) the light chain sequence set forth in SEQ ID NO: 44, and the heavy chain sequence set forth in SEQ ID NO: 49.

51. The bioactivatable polypeptide drug construct according to claim 48, wherein domain D2 is an IL-15 variant polypeptide selected from the group of polypeptides having the amino acid sequence set forth in SEQ ID NOs: 117-163.

52. The bioactivatable polypeptide drug construct according to claim 48, wherein domain D3 is an IL-15Rα sushi variant polypeptide having the amino acid sequence set forth in SEQ ID NO: 165.

53. The bioactivatable polypeptide drug construct according to claim 48, wherein the construct is selected from the group of constructs wherein (a) the D1, D2 and D3 domains of the construct are each in the form of a monomer, or (2) the D1, D2 and D3 domains of the construct are each in the form of a dimer.

54. The bioactivatable polypeptide drug construct according to claim 48, wherein D2 is attached to D1 by a peptide linker (“L1”) selected from the group consisting of a protease cleavable peptide linker and a non-cleavable peptide linker, wherein the protease cleavable peptide linker is selected from the group of sequences set forth in SEQ ID NOs: 54-77 and 78-94, and wherein the non-cleavable peptide linker is selected from the group of sequences set forth in SEQ ID NOs: 95-115.

55. The bioactivatable polypeptide drug construct according to claim 48, wherein D2 is attached to D3 by a peptide linker (“L2”) selected from the group consisting of a protease cleavable peptide linker and a non-cleavable peptide linker, wherein the protease cleavable peptide linker is selected from the group of sequences set forth in SEQ ID NOs: 54-77 and 78-94, and wherein the non-cleavable peptide linker is selected from the group of sequences set forth in SEQ ID NOs: 95-115.

56. A pharmaceutical composition comprising a bioactivatable polypeptide drug construct according to claim 55 in admixture with a pharmaceutically acceptable carrier.

57. A method for treating cancer or cancer metastasis in a subject, comprising administering to the subject a therapeutically effective amount of the pharmaceutical composition according to claim 56, wherein the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.


SEQUENCE LISTING (185 sequences)

SEQ ID NO: 1 - protein, 530 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPLFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 112 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 3 - protein, 112 aa, synthetic construct
EIVLTQSPATLSLSPGERATLSCRASQGVSTSGYSYLHWYQQKPGQAPRLLIYLASYRESGVPARFSGSGSGTDFTLTISSLEPEDFAVYYCQHSRDLPLTFGGGTKVEIKR

SEQ ID NO: 4 - protein, 112 aa, synthetic construct
EIVLTQSPATLSLSPGERATLSCRASQGVSTSGYSYLHWYQQKPGQAPRLLIYLASYRASGVPARFSGSGSGTDFTLTISSLEPEDFAVYYCQHSRDLPLTFGGGTKVEIKR

SEQ ID NO: 5 - protein, 112 aa, synthetic construct
EIVLTQSPATLSLSPGERATLSCRASQGVSTSGYSYLAWYQQKPGQAPRLLIYLASYRASGVPARFSGSGSGTDFTLTISSLEPEDFAVYYCQHSRDLPLTFGGGTKVEIKR

SEQ ID NO: 6 - protein, 120 aa, synthetic construct [sequence omitted]

SEQ ID NO: 7 - protein, 120 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSS

SEQ ID NO: 8 - protein, 120 aa, synthetic construct
QVQLVQSGVEVKKPGASVKVSCKASGYTFTNYYMYWVRQAPGQGLEWMGGINPSNGGTNYAQKFQGRVTLTTDSSTTTAYMELKSLQFDDTAVYYCARRDYRFDMGFDYWGQGTTVTVSS

SEQ ID NO: 9 - protein, 120 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSS

SEQ ID NO: 10 - protein, 120 aa, synthetic construct
EVQLLESGGGLVQPGGSLRLSCAASGFTFTNYYMYWVRQAPGKGLEWVSGINPSNGGTNYADKFKGRFTISRDNSKNTLYLQMNSLRAEDTAVYYCARRDYRFDMGFDYWGQGTLVTVSS

SEQ ID NO: 11 - protein, 120 aa, synthetic construct
EVQLLESGGGLVQPGGSLRLSCAASGFTFTNYYMYWVRQAPGKGLEWVSGINPSNGGTNYADKFKGRFTLSTDSSKNTLYLQMNSLRAEDTAVYYCARRDYRFDMGFDYWGQGTLVTVSS

SEQ ID NO: 12 - protein, 120 aa, synthetic construct
EVQLLESGGGLVQPGGSLRLSCAASGFTFTNYYMYWVRQAPGKGLEWMGGINPSNGGTNYADKFKGRFTISRDNSKNTLYLQMNSLRAEDTAVYYCARRDYRFDMGFDYWGQGTLVTVSS

SEQ ID NO: 13 - protein, 120 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSS

SEQ ID NO: 14 - protein, 120 aa, synthetic construct
EVQLLESGGGLVQPGGSLRLSCAASGFTFTNYYMYWVRQAPGKGLEWVSGINPSNGGTNFNDSVKGRFTISRDNSKNTLYLQMNSLRAEDTAVYYCARRDYRFDMGFDYWGQGTLVTVSS

SEQ ID NO: 15 - protein, 120 aa, synthetic construct
EVQLLESGGGLVQPGGSLRLSCAASGFTFTNYYMYWVRQAPGKGLEWVSGINPSNGGTNFADKFKGRFTISRDNSKNTLYLQMNSLRAEDTAVYYCARRDYRFDMGFDYWGQGTLVTVSS

SEQ ID NO: 16 - protein, 120 aa, synthetic construct
EVQLLESGGGLVQPGGSLRLSCAASGFTFTNYYMYWVRQAPGKGLEWVSGINPSNGGTNFADKFKGRFTISRDSSKNTLYLQMNSLRAEDTAVYYCARRDYRFDMGFDYWGQGTLVTVSS

SEQ ID NO: 17 - protein, 120 aa, synthetic construct
EVQLLESGGGLVQPGGSLRLSCAASGFTFTNYYMYWVRQAPGKGLEWVSGINPSNGGTNFADKFKGRFTISTDSSKNTLYLQMNSLRAEDTAVYYCARRDYRFDMGFDYWGQGTLVTVSS

SEQ ID NO: 18 - protein, 120 aa, synthetic construct
EVQLLESGGGLVQPGGSLRLSCAASGFTFTNYYMYWVRQAPGKGLEWVSGINPSNGGTNFADKFKGRFTLSTDSSKNTLYLQMNSLRAEDTAVYYCARRDYRFDMGFDYWGQGTLVTVSS

SEQ ID NO: 19 - protein, 15 aa, synthetic construct
RASKGVSTSGYSYLH

SEQ ID NO: 20 - protein, 15 aa, synthetic construct
RASQGVSTSGYSYLH

SEQ ID NO: 21 - protein, 15 aa, synthetic construct
RASQGVSTSGYSYLA

SEQ ID NO: 22 - protein, 8 aa, synthetic construct
YLASYLES

SEQ ID NO: 23 - protein, 8 aa, synthetic construct
YLASYRES

SEQ ID NO: 24 - protein, 8 aa, synthetic construct
YLASYRAS

SEQ ID NO: 25 - protein, 9 aa, synthetic construct
QHSRDLPLT

SEQ ID NO: 26 - protein, 5 aa, synthetic construct
NYYMY

SEQ ID NO: 27 - protein, 17 aa, synthetic construct
GINPSNGGTNFNEKFKN

SEQ ID NO: 28 - protein, 17 aa, synthetic construct
GINPSNGGTNFAQKFQG

SEQ ID NO: 29 - protein, 17 aa, synthetic construct
GINPSNGGTNYAQKFQG

SEQ ID NO: 30 - protein, 17 aa, synthetic construct
GINPSNGGTNYADKFKG

SEQ ID NO: 31 - protein, 17 aa, synthetic construct
GINPSNGGTNFADKFKG

SEQ ID NO: 32 - protein, 17 aa, synthetic construct
GINPSNGGTNFNDSVKG

SEQ ID NO: 33 - protein, 11 aa, synthetic construct
RDYRFDMGFDY

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 38 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 39 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 40 - protein, 122 aa, Homo sapiens
QVQLVQSGVEVKKPGASVKVSCKASGYTFTSNAISWVRQAPGQGLEWMGWISTYKGKANYAQKFQDRVTLTTDSSTTTAYMELKSLQFDDTAVYYCARWRAVVGRGGGLDVWGQGTTVTVSS

SEQ ID NO: 41 - protein, 108 aa, Homo sapiens
EIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 42 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 449 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 218 aa, synthetic construct
EIVLTQSPATLSLSPGERATLSCRASQGVSTSGYSYLHWYQQKPGQAPRLLIYLASYRESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 449 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 449 aa, synthetic construct
EVQLLESGGGLVQPGGSLRLSCAASGFTFTNYYMYWVRQAPGKGLEWVSGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 449 aa, synthetic construct
EVQLLESGGGLVQPGGSLRLSCAASGFTFTNYYMYWVRQAPGKGLEWVSGINPSNGGTNFADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 449 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 449 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 214 aa, synthetic construct
DIVMTQSPLSLPVTPGEPASITCKASQDVETVVAWYLQKPGQSPRLLIYWASTRHTGVPDRFSGSGSGTDFTLKISRVEAEDVGVYYCQQYSRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 446 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 218 aa, Mus musculus
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 53 - protein, 444 aa, Mus musculus
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 54 - protein, 8 aa, synthetic construct
SPLGLAGS

SEQ ID NO: 55 - protein, 8 aa, synthetic construct
EPLELRAG

SEQ ID NO: 56 - protein, 8 aa, synthetic construct
LSGRSDNH

SEQ ID NO: 57 - protein, 8 aa, synthetic construct
GPLGIAGQ

SEQ ID NO: 58 - protein, 8 aa, synthetic construct
GTAHLMGG

SEQ ID NO: 59 - protein, 8 aa, synthetic construct
RIGSLRTA

SEQ ID NO: 60 - protein, 10 aa, synthetic construct
SGRSENIRTA

SEQ ID NO: 61 - protein, 8 aa, synthetic construct
GPLGMLSQ

SEQ ID NO: 62 - protein, 8 aa, synthetic construct
GPAGMKGL

SEQ ID NO: 63 - protein, 8 aa, synthetic construct
RPSASRSA

SEQ ID NO: 64 - protein, 6 aa, synthetic construct
PLGLAG

SEQ ID NO: 65 - protein, 13 aa, synthetic construct
LGGSGRSANAILE

SEQ ID NO: 66 - protein, 10 aa, synthetic construct
GGSGRSANAI

SEQ ID NO: 67 - protein, 5 aa, synthetic construct
SGRSA

SEQ ID NO: 68 - protein, 4 aa, synthetic construct
AANL

SEQ ID NO: 69 - protein, 7 aa, synthetic construct
GPTNKVR

SEQ ID NO: 70 - protein, 4 aa, synthetic construct
GFFY

SEQ ID NO: 71 - protein, 8 aa, synthetic construct
GPICFRLG

SEQ ID NO: 72 - protein, 7 aa, synthetic construct
RQAGFSL

SEQ ID NO: 73 - protein, 8 aa, synthetic construct
RQARAVGG

SEQ ID NO: 74 - protein, 5 aa, synthetic construct
PMAKK

SEQ ID NO: 75 - protein, 6 aa, synthetic construct
HSSKLQ

SEQ ID NO: 76 - protein, 13 aa, synthetic construct
GPLGMLSQPMAKK

SEQ ID NO: 77 - protein, 13 aa, synthetic construct
PMAKKGPLGMLSQ

SEQ ID NO: 78 - protein, 30 aa, synthetic construct
GGGSGGGGSGGGGSLSGRSDNHGGSGGGGS

SEQ ID NO: 79 - protein, 15 aa, synthetic construct
GSSSGRSENIRTAGT

SEQ ID NO: 80 - protein, 35 aa, synthetic construct
GGGGSGGGGSGGGSLGGSGRSANAILEGGSGGGGS

SEQ ID NO: 81 - protein, 28 aa, synthetic construct
GGGGSGGGGSLGGSGRSANAILEGGGGS

SEQ ID NO: 82 - protein, 21 aa, synthetic construct
GGGGSLGGSGRSANAILEGGS

SEQ ID NO: 83 - protein, 14 aa, synthetic construct
GGGSGPTNKVRGGS

SEQ ID NO: 84 - protein, 15 aa, synthetic construct
GGSGPLGMLSQGGGS

SEQ ID NO: 85 - protein, 10 aa, synthetic construct
GGPLGMLSQS

SEQ ID NO: 86 - protein, 13 aa, unidentified
GGGPLGMLSQGGS

SEQ ID NO: 87 - protein, 10 aa, synthetic construct
GGPTNKVRGS

SEQ ID NO: 88 - protein, 10 aa, synthetic construct
GRQARAVGGS

SEQ ID NO: 89 - protein, 15 aa, synthetic construct
GGGSGRSENIRTAGG

SEQ ID NO: 90 - protein, 15 aa, synthetic construct
SGGPGPAGMKGLPGS

SEQ ID NO: 91 - protein, 15 aa, synthetic construct
GGGGSPMAKKGGGGS

SEQ ID NO: 92 - protein, 15 aa, synthetic construct
GGPLGMLSQPMAKKS

SEQ ID NO: 93 - protein, 20 aa, synthetic construct
GGSGPLGMLSQPMAKKGGGS

SEQ ID NO: 94 - protein, 20 aa, synthetic construct
GGGPMAKKGPLGMLSQGGGS

SEQ ID NO: 95 - protein, 14 aa, synthetic construct
EPKSSDKTHTSPPS

SEQ ID NO: 96 - protein, 12 aa, synthetic construct
GGGSGGGSGGGS

SEQ ID NO: 97 - protein, 4 aa, synthetic construct
GGGS

SEQ ID NO: 98 - protein, 9 aa, synthetic construct
GSSGGSGGS

SEQ ID NO: 99 - protein, 5 aa, synthetic construct
GSSGT

SEQ ID NO: 100 - protein, 15 aa, synthetic construct
GGGGSGGGGSGGGGS

SEQ ID NO: 101 - protein, 17 aa, synthetic construct
AEAAAKEAAAKEAAAKA

SEQ ID NO: 102 - protein, 20 aa, synthetic construct
GGGGSGGGGSGGGGSGGGGS

SEQ ID NO: 103 - protein, 8 aa, synthetic construct
GGGSGGGS

SEQ ID NO: 104 - protein, 4 aa, synthetic construct
GSGG

SEQ ID NO: 105 - protein, 4 aa, synthetic construct
GGSS

SEQ ID NO: 106 - protein, 5 aa, synthetic construct
GGGGS

SEQ ID NO: 107 - protein, 5 aa, synthetic construct
GGSGG

SEQ ID NO: 108 - protein, 4 aa, synthetic construct
SGGG

SEQ ID NO: 109 - protein, 4 aa, synthetic construct
GSGS

SEQ ID NO: 110 - protein, 6 aa, synthetic construct
GSGSGS

SEQ ID NO: 111 - protein, 8 aa, synthetic construct
GSGSGSGS

SEQ ID NO: 112 - protein, 10 aa, synthetic construct
GSGSGSGSGS

SEQ ID NO: 113 - protein, 12 aa, synthetic construct
GSGSGSGSGSGS

SEQ ID NO: 114 - protein, 10 aa, synthetic construct
GGGGSGGGGS

SEQ ID NO: 115 - protein, 15 aa, synthetic construct
GGGGSGGGGSGGGGS

SEQ ID NO: 116 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 117 - protein, 114 aa, synthetic construct
NWVNVISDLKKIEDLIQSMHIDATLYTESDVHPSCKVTAMKCFLLELQVISLESGDADIHDTVENLIILANNSLSSNGNVTESGCKECEELEEKNIKEFLQSFVHIVQMFINTS

SEQ ID NO: 118 - protein, 113 aa, synthetic construct
WVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 119 - protein, 112 aa, synthetic construct
VNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 120 - protein, 111 aa, synthetic construct
NVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 121 - protein, 114 aa, synthetic construct
NWVNVISDLKKIEDLIQSMHIDATLYTESDVHPSCKVTAMKCFLLELQVISLESGDASIHDTAENLIILANNSLSSNGNVTESGCKECEELEEKNIKEFLQSFVHIVQMFINTS

SEQ ID NO: 122 - protein, 114 aa, synthetic construct
NWVNVISDLKKIEDLIQSMHIDATLYTESDVHPSCKVTAMKCFLLELQVISLESGDASIHDTFENLIILANNSLSSNGNVTESGCKECEELEEKNIKEFLQSFVHIVQMFINTS

SEQ ID NO: 123 - protein, 114 aa, synthetic construct
NWVNVISDLKKIEDLIQSMHIDATLYTESDVHPSCKVTAMKCFLLELQVISLESGDASIHDTKENLIILANNSLSSNGNVTESGCKECEELEEKNIKEFLQSFVHIVQMFINTS

SEQ ID NO: 124 - protein, 114 aa, synthetic construct
NWVNVISDLKKIEDLIQSMHIDATLYTESDVHPSCKVTAMKCFLLELQVISLESGDASIHDTRENLIILANNSLSSNGNVTESGCKECEELEEKNIKEFLQSFVHIVQMFINTS

SEQ ID NO: 125 - protein, 114 aa, synthetic construct
NWVNVISDLKKIEDLIQSMHIDATLYTESDVHPSCKVTAMKCFLLELQVISLESGDASIHDTVENLIDLANNSLSSNGNVTESGCKECEELEEKNIKEFLQSFVHIVQMFINTS

SEQ ID NO: 126 - protein, 114 aa, synthetic construct
NWVNVISDLKKIEDLIQSMHIDATLYTESDVHPSCKVTAMKCFLLELQVISLESGDASIHDTVENLIFLANNSLSSNGNVTESGCKECEELEEKNIKEFLQSFVHIVQMFINTS

SEQ ID NO: 127 - protein, 114 aa, synthetic construct
NWVNVISDLKKIEDLIQSMHIDATLYTESDVHPSCKVTAMKCFLLELQVISLESGDASIHDTVENLIGLANNSLSSNGNVTESGCKECEELEEKNIKEFLQSFVHIVQMFINTS

SEQ ID NO: 128 - protein, 114 aa, synthetic construct
NWVNVISDLKKIEDLIQSMHIDATLYTESDVHPSCKVTAMKCFLLELQVISLESGDASIHDTVENLIHLANNSLSSNGNVTESGCKECEELEEKNIKEFLQSFVHIVQMFINTS

SEQ ID NO: 129 - protein, 114 aa, synthetic construct
NWVNVISDLKKIEDLIQSMHIDATLYTESDVHPSCKVTAMKCFLLELQVISLESGDASIHDTVENLIKLANNSLSSNGNVTESGCKECEELEEKNIKEFLQSFVHIVQMFINTS

SEQ ID NO: 130 - protein, 114 aa, synthetic construct
NWVNVISDLKKIEDLIQSMHIDATLYTESDVHPSCKVTAMKCFLLELQVISLESGDASIHDTVENLIQLANNSLSSNGNVTESGCKECEELEEKNIKEFLQSFVHIVQMFINTS

SEQ ID NO: 131 - protein, 114 aa, synthetic construct
NWVNVISDLKKIEDLIQSMHIDATLYTESDVHPSCKVTAMKCFLLELQVISLESGDASIHDTAENLIGLANNSLSSNGNVTESGCKECEELEEKNIKEFLQSFVHIVQMFINTS

SEQ ID NO: 132 - protein, 114 aa, synthetic construct
NWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTS

SEQ ID NO: 133 - protein, 114 aa, synthetic construct
NWVNVISDLKKIEDLIQSMHIDATLYTESDVHPSCKVTAMKCFLLELQVISLESGDASIHDTAENLIQLANNSLSSNGNVTESGCKECEELEEKNIKEFLQSFVHIVQMFINTS

SEQ ID NO: 134 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVAMFINTS

SEQ ID NO: 135 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVDMFINTS

SEQ ID NO: 136 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 137 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVFMFINTS

SEQ ID NO: 138 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVHMFINTS

SEQ ID NO: 139 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVKMFINTS

SEQ ID NO: 140 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVLMFINTS

SEQ ID NO: 141 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVMMFINTS

SEQ ID NO: 142 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVNMFINTS

SEQ ID NO: 143 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVSMFINTS

SEQ ID NO: 144 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVTMFINTS

SEQ ID NO: 145 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVYMFINTS

SEQ ID NO: 146 - protein, 114 aa, synthetic construct
NWVNVISDLKKIEDLIQSMHIDATLYTESDVHPSCKVTAMKCFLLELQVISLESGDASIHDTAENLIILANNSLSSNGNVTESGCKECEELEEKNIKEFLQSFVHIVMMFINTS

SEQ ID NO: 147 - protein, 114 aa, synthetic construct
NWVNVISDLKKIEDLIQSMHIDATLYTESDVHPSCKVTAMKCFLLELQVISLESGDASIHDTKENLIILANNSLSSNGNVTESGCKECEELEEKNIKEFLQSFVHIVMMFINTS

SEQ ID NO: 148 - protein, 114 aa, synthetic construct
NWVNVISDLKKIEDLIQSMHIDATLYTESDVHPSCKVTAMKCFLLELQVISLESGDASIHDTVENLIFLANNSLSSNGNVTESGCKECEELEEKNIKEFLQSFVHIVMMFINTS

SEQ ID NO: 149 - protein, 114 aa, synthetic construct
NWVNVISDLKKIEDLIQSMHIDATLYTESDVHPSCKVTAMKCFLLELQVISLESGDASIHDTVENLIHLANNSLSSNGNVTESGCKECEELEEKNIKEFLQSFVHIVMMFINTS

SEQ ID NO: 150 - protein, 114 aa, synthetic construct
NWVNVISDLKKIEDLIQSMHIDATLYTESDVHPSCKVTAMKCFLLELQVISLESGDASIHDTKENLIILANNSLSSNGNVTESGCKECEELEEKNIKEFLQSFVHIVNMFINTS

SEQ ID NO: 151 - protein, 114 aa, synthetic construct
NWVNVISDLKKIEDLIQSMHIDATLYTESDVHPSCKVTAMKCFLLELQVISLESGDASIHDTVENLIHLANNSLSSNGNVTESGCKECEELEEKNIKEFLQSFVHIVNMFINTS

SEQ ID NO: 152 - protein, 114 aa, synthetic construct
NWVNVISDLKKIEDLIQSMHIDATLYTESTVHPSCKVTAMKCFLLELQVISLESGDASIHDTVENLIILANNSLSSNGNVTESGCKECEELEEKNIKEFLQSFVHIVQMFINTS

SEQ ID NO: 153 - protein, 114 aa, synthetic construct
NWVNVISDLKKIEDLIQSMHIDATLYTESDVEPSCKVTAMKCFLLELQVISLESGDASIHDTVENLIILANNSLSSNGNVTESGCKECEELEEKNIKEFLQSFVHIVQMFINTS

SEQ ID NO: 154 - protein, 114 aa, synthetic construct
NWVNVISDLKKIEDLIQSMHIDATLYTESDVDPSCKVTAMKCFLLELQVISLESGDASIHDTVENLIILANNSLSSNGNVTESGCKECEELEEKNIKEFLQSFVHIVQMFINTS

SEQ ID NO: 155 - protein, 114 aa, synthetic construct
NWVNVISDLKKIEDLIQSMHIDATLYTESDVNPSCKVTAMKCFLLELQVISLESGDASIHDTVENLIILANNSLSSNGNVTESGCKECEELEEKNIKEFLQSFVHIVQMFINTS

SEQ ID NO: 156 - protein, 114 aa, synthetic construct
NWVNVISDLKKIEDLIQSMHIDATLYTESDVQPSCKVTAMKCFLLELQVISLESGDASIHDTVENLIILANNSLSSNGNVTESGCKECEELEEKNIKEFLQSFVHIVQMFINTS

SEQ ID NO: 157 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQAFINTS

SEQ ID NO: 158 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQHFINTS

SEQ ID NO: 159 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQRFINTS

SEQ ID NO: 160 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIDTS

SEQ ID NO: 161 - protein, 114 aa, synthetic construct; identical to SEQ ID NO: 160

SEQ ID NO: 162 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIRTS

SEQ ID NO: 163 - protein, 114 aa, synthetic construct; identical to SEQ ID NO: 162

SEQ ID NO: 164 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 165 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 166 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 167 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 169 - protein, 443 aa, Mus musculus
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 170 - protein, 443 aa, Mus musculus
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 171 - protein, 443 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWDGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 443 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWDGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 173 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 449 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 175 - protein, 575 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTS

SEQ ID NO: 176 - protein, 573 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 177 - protein, 578 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTS

SEQ ID NO: 178 - protein, 578 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTS

SEQ ID NO: 179 - protein, 578 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVNMFINTS

SEQ ID NO: 180 - protein, 578 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTVENLIHLANNSLSSNGNVTESGCKECEELEEKNIKEFLQSFVHIVNMFINTS

SEQ ID NO: 181 - protein, 662 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTSGGPLGMLSQSITCPPPMSVEHADIWVKSYSLYSRERYICNSGFKRKAGTSSLTECVLNKATNVAHWTTPSLKCIRDPALVHQRPAPP

SEQ ID NO: 182 - protein, 675 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTSGGSGPLGMLSQPMAKKGGGSITCPPPMSVEHADIWVKSYSLYSRERYICNSGFKRKAGTSSLTECVLNKATNVAHWTTPSLKCIRDPALVHQRPAPP

SEQ ID NO: 183 - protein, 675 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTAENLIHLANNSLSSNGNVTESGCKECEELEEKNIKEFLQSFVHIVQMFINTSGGSGPLGMLSQPMAKKGGGSITCPPPMSVEHADIWVKSYSLYSRERYICNSGFKRKAGTSSLTECVLNKATNVAHWTTPSLKCIRDPALVHQRPAPP

SEQ ID NO: 184 - protein, 675 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVNMFINTSGGSGPLGMLSQPMAKKGGGSITCPPPMSVEHADIWVKSYSLYSRERYICNSGFKRKAGTSSLTECVLNKATNVAHWTTPSLKCIRDPALVHQRPAPP

SEQ ID NO: 185 - protein, 675 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVNMFINTSGGSGPLGMLSQPMAKKGGGSITCPPPMSVEHADIWVKSYSLYSRERYICNSGFKRKAGTSSLTECVLNKATNVAHWTTPSLKCIRDPALVHQRPAPP